SEQUENCE LISTING (2 sequences)

SEQ ID NO: 1 - DNA, 28 bp, Artificial
  Primer
ggggacaactttgtacaaaaaagttggc

SEQ ID NO: 2 - DNA, 28 bp, Artificial
  Primer
gagagttagtggcccgcaggtcggggga

(19) United States
(12) Patent Application Publication
(10) Pub. No.: US 2012/0090043 A1
(43) Pub. Date: Apr. 12, 2012

(54) TARGETS FOR RETROVIRUS ASSOCIATED DISEASES

(75) Inventors: Jean-Claude Twizere, Gembloux (BE); Nicolas Simonis, Bruxelles (BE)

(73) Assignee: UNIVERSITE DE LIEGE, Liege (BE)

(21) Appl. No.: 13/378,454

(22) PCT Filed: Jun. 30, 2010
(86) PCT No.: PCT/EP10/59266

§ 371 Date: Dec. 15, 2011

(30) Foreign Application Priority Data
Jun. 30, 2009 (EP) 09164241.3

(51) Int. Cl. C07K 19/00; C12N 15/62; C12N 15/63; C12N 1/21; C12N 1/19; C12N 1/15; C12N 1/11; C12N 5/10; A01H 5/00; A01K 67/00; A01K 67/027; A61K 39/21; C07K 16/18; C07K 16/40; C07K 14/00; C07K 2/00; C07H 21/00; G01N 33/566; C07H 21/02; A61K 39/395; A61K 38/02; A61K 31/7088; A61K 31/713; C12Q 1/70; C40B 30/06; A61P 37/04; A61P 31/14; A61P 31/18; C12N 9/96

(52) U.S. Cl. 800/14; 530/350; 435/188; 536/23.2; 536/23.4; 435/320.1; 435/252.3; 435/254.2; 435/254.11; 435/258.1; 435/252.33; 435/252.31; 435/254.21; 435/254.23; 435/419; 435/414; 435/325; 435/363; 435/366; 435/348; 800/298; 800/8; 800/13; 424/192.1; 530/389.4; 530/300; 536/23.1; 436/501; 536/24.5; 424/158.1; 424/159.1; 514/3.7; 514/44 R; 514/44 A; 435/5; 506/10

(57) ABSTRACT

The invention concerns an isolated complex comprising an HIV or HTLV protein and a human protein. Corresponding nucleic acids, vectors, host cells, host organisms, compositions, kits, medical uses, diagnostic uses, and methods of screening agents are also contemplated. Disclosed are 212 interactions between 19 retroviral proteins and 131 human proteins.

### FIELD OF THE INVENTION

The invention generally relates to retrovirus associated diseases, including diseases caused by human immunodeficiency virus (HIV) or human T-lymphotropic virus (HTLV). In particular, the invention discloses host proteins targeted by viral proteins in retrovirus associated diseases, and teaches related products and methods useful for the study, assessment and treatment of said diseases.

### BACKGROUND OF THE INVENTION

The importance of devising new or improved manners to combat retrovirus associated diseases, particularly diseases caused by human immunodeficiency virus (HIV) or human T-lymphotropic virus (HTLV), is widely acknowledged.

Human pathogenic retroviruses particularly include HIV type 1 (HIV-1) and type 2 (HIV-2) and HTLV type-1 (HTLV-1). HIV and HTLV both target T-lymphocytes but produce different disease outcomes. HIV invades CD4+ T-helper lymphocytes and causes severe defects in cell-mediated immune responses characteristic of acquired immunodeficiency syndrome (AIDS). In contrast, HTLV-1 does not destroy T-cells, but induces adult T-cell leukaemia/lymphoma (ATLL), an aggressive lymphoproliferative disease. HTLV-1 is also associated with tropical spastic paraparesis (TSP), a neurological degenerative syndrome. HTLV-2, which is closely related to HTLV-1, does not cause any known disease phenotype.

HIV and HTLV genomes encode structural proteins which contribute to the viral core particle (Gag and Env) and enzymatic retroviral proteins (namely reverse transcriptase, integrase and protease). Both HIV and HTLV further comprise a cluster of alternatively spliced open reading frames encoding regulatory proteins (Tat, Rev, Nef, Vif, Vpr, Vpu and Vpx for HIV; and Tax, Rex, HBZ, p30, p13 and p12 for HTLV).

Several earlier studies looked for human factors interacting with one or more of the above retroviral proteins (Fu et al. 2009. Nucleic Acids Res 37: D417-422; Boxus et al. 2008. Retrovirology 5: 76; Chatr-aryamontri et al. 2009. Nucleic Acids Res 37: D669-673; Navratil et al. 2009. Nucleic Acids Res 37: D661-668). However, most data has been generated for HIV-1 virus, whereas the interactomes of HIV-2, HTLV-1 and HTLV-2 viruses have been only sparsely investigated.

The present Applicant postulates that additional, thus far unknown host factors and pathways are exploited by retroviruses to cause disease and represent valuable targets for the treatment of retrovirus-induced pathologies. The invention answers the need to systematically identify and characterise further targets of pathogenic retroviruses, and to provide useful products and methods relying on such targets.

### SUMMARY OF THE INVENTION

Having conducted extensive experiments and tests, the present Applicant has identified host proteins interacting with retroviral proteins, in particular with proteins of HIV-1, HIV-2, HTLV-1 or HTLV-2 viruses. The herein taught host proteins provide important targets for modulating retrovirus associated diseases and conditions.

In particular, using a systematic unbiased binary interactome mapping strategy, 212 interactions have been confirmed involving 19 retroviral proteins and 131 human proteins (Table 1). Among these 212 interactions, 28, 26, 87 and 71 interactions involved proteins encoded by HIV-1, HIV-2, HTLV-1 and HTLV-2, respectively. The Applicant has further thoroughly analysed the retrovirus-host protein interaction profiles, and specified preferred subgroups of host target proteins playing a role in diseases and conditions caused by the corresponding retroviruses.

Table 1 shows human proteins identified in the present application as interacting with proteins encoded by retroviral open reading frames (ORF).

| Viral ORF | Gene symbols of human proteins interacting with the viral proteins |
|---|---|
| HIV1_gagpol | DIPA, HSFY1, SS18L1 |
| HIV1_gag | DLAT, DLX2, HSFY1, KCTD1, KLHL12, LNX2, TRAF2 |
| HIV1_tat | BACH1, HOXA3, HOXD3, L3MBTL3, LDOC1, LNX2, LOC541468, |
| | MIZF, PDE9A, SF3A3 |
| HIV1_rev | MIZF |
| HIV1_env | HSFY1, SFRS11 |
| HIV1_nef | C8orf56, CDC23, CRX, HSFY1, LNX2 |
| HIV2_gagpol | DIPA, SS18L1, HSFY1 |
| HIV2_gag | DLAT, DLX2, HSFY1, KCTD1, KLHL12, LNX2, SS18L1, TRAF2 |
| HIV2_tat | BACH1, FKBP7, FLJ20097, HOXA3, HOXD3, KRT6A, L3MBTL3, |
| | LDOC1, LNX2, LOC541468, MIZF, PDE9A, PLEKHF2, SF3A3 |
| HIV2_rev | MIZF |
| HTLV1_rex | BHLHB2, C10orf3, CRX, Dlc2, DVL2, FLJ25439, LNX2, LOC391257, |
| | LZTS2, NIF3L1, NUP62, PIAS2, RIBC2, SP100, SPAG5, SPG21, UBE2I, |
| | ZCCHC7 |
| HTLV1_tax | C16orf33, C1orf59, C20orf141, CCDC24, CDC23, Cep63, CHCHD3, |
| | CRADD, CRX, CTDSP2, CTNNBIP1, DGCR6L, DHRS10, ETV4, |
| | EWSR1, FANCG, FATE1, FLJ22471, FLJ25439, FLJ32855, HGS, |
| | HOXB9, KIAA0258, KIAA1949, KIF9, KRT15, KRT4, KRT6A, KRT8, |
| | LENG1, LNX2, LOC283385, LOC388818, LOC595101, LOC91661, |
| | MAD2L2, MINA, MRPS6, MYEF2, NEFL, NIF3L1, NOS3, NUDT18, |
| | POLM, POP5, PSMA1, RFX4, RIBC2, SPG21, TRIP6, TTC23, VPS28, |
| | WDFY3, ZMAT1, ZNF581, ZNF614, ZNHIT4 |
| HTLV1_env | TSC22D4, C20orf81 |
| HTLV1_hbz | DAZAP2, FTH1, GOPC, HOXD3, MLLT11, MLX, MYST2, OTX2, |
| | PCBP1, RNPS1 |
| HTLV2_gag | DLX2, KCTD1, KLHL12, LNX2, TFIP11, TRAF2 |
| HTLV2_pol | KLC3, KRT15, MEIS2, MKRN3, RBPMS, SORBS3, TEX11, TFIP11 |
| HTLV2_rex | CRX, Dlc2, LNX2, NIF3L1, NUP62, SP100, SPG21 |
| HTLV2_tax2 | AP4M1, ARL6IP4, BHLHB2, C1orf94, C8orf32, CCDC24, CDC23, |
| | Cep63, Cep70, CRX, DDX6, DGCR6L, Dlc2, EWSR1, FATE1, |
| | FLJ10726, FLJ22471, FLJ32855, FXR2, GADD45GIP1, GCC1, HGS, |
| | KIAA1683, KRT15, LNX2, LOC388818, LOC595101, LZTS2, MAD1L1, |
| | MGC11257, MGC27019, MGC4266, MINA, MRPS6, NIF3L1, NKAP, |
| | NUDT18, NUP62, POLM, PSMF1, SLC2A4, SOX5, SP100, SPG21, |
| | SSX2IP, VPS28, ZBTB16, ZNF581, ZNF614 |
| HTLV2_env | CRSP9 |

Consequently, an aspect of the invention provides an isolated complex comprising, consisting substantially of or consisting of a first and second proteins, wherein:
the first protein is a protein of a retrovirus, preferably a protein of a human retrovirus including human pathogenic and non-pathogenic retrovirus, more preferably a protein of the human immunodeficiency virus (HIV) or of the human T-cell leukaemia virus (HTLV), or a functional fragment or variant of such a protein; and
the second protein is selected from TRAF2, AP4M1, ARL61P4, BACH1, BHLHB2, C10orf3, C16orf33, Clorf59, Clorf94, C20orf141, C20orf81, C8orf32, C8orf56, CCDC24, Cep63, Cep70, CHCHD3, CRADD, CRSP9, CRX, CTDSP2, CTNNBIP1, DAZAP2, DDX6, DGCR6L, DHRS10, DIPA, DLAT, D1c2, DLX2, DVL2, ETV4, EWSR1, FANCG, FATE1, FKBP7, FLJ10726, FLJ20097, FLJ22471, FLJ25439, FLJ32855, FTH1, FXR2, GADD45GIP1, GCC1, GOPC, HGS, HOXA3, HOXB9, HOXD3, HSFY1, KCTD1, KIAA0258, KIAA1683, KIAA1949, KIF9, KLC3, KLHL12, KRT15, KRT4, KRT6A, L3 MBTL3, LDOC1, LENG1, LNX2, LOC283385, LOC388818, LOC391257, LOC541468, LOC595101, LOC91661, LZTS2, MAD2L2, MEIS2, MGC11257, MGC27019, MGC4266, MINA, MIZF, MKRN3, MLLT11, MLX, MRPS6, MYEF2, MYST2, NEFL, NIF3L1, NKAP, NOS3, NUDT18, OTX2, PCBP1, PDE9A, PIAS2, PLEKHF2, POLM, POPS, RBPMS, RFX4, RIBC2, RNPS1, SF3A3, SFRS11, SLC2A4, SORBS3, SOX5, SP100, SPAG5, SPG21, SS18L1, SSX21P, TEX11, TFIP11, TRIP6, TSC22D4, TTC23, VPS28, WDFY3, ZBTB16, ZCCHC7, ZMAT1, ZNF581, ZNF614, ZNHIT4 or a functional fragment or variant of any one thereof.

Also provided is an isolated complex comprising, consisting substantially of or consisting of a first and second proteins, wherein:
the first protein is a protein of a retrovirus, preferably a protein of a human retrovirus including human pathogenic and non-pathogenic retrovirus, more preferably a protein of HIV or of HTLV, even more preferably an HIV or HTLV protein as listed in Table 1 above, or a functional fragment or variant of such a protein; and
the second protein is selected from proteins listed in second column of Table 1 above, or a functional fragment or variant of any one thereof.

In a particular aspect the invention provides an isolated complex comprising, consisting substantially of or consisting of a first and second proteins, wherein:
the first protein is a protein of the human immunodeficiency virus (HIV) or a functional fragment or variant of an HIV protein; and
the second protein is selected from TRAF2, BACH1, C8orf56, CDC23, CRX, DIPA, DLAT, DLX2, FKBP7, FLJ20097, HOXA3, HOXD3, HSFY1, KCTD1, KLHL12, KRT6A, L3 MBTL3, LDOC1, LNX2, LOC541468, MIZF, PDE9A, PLEKHF2, SF3A3, SFRS11, SS18L1 or a functional fragment or variant of any one thereof.

Another particular aspect provides an isolated complex comprising, consisting substantially of or consisting of a first and second proteins, wherein:
the first protein is a protein of the human T-cell leukaemia virus (HTLV) or a functional fragment or variant of an HTLV protein; and
the second protein is selected from TRAF2, AP4M1, ARL61P4, BHLHB2, C10orf3, C16orf33, Clorf59, Clorf94, C20orf141, C20orf81, C8orf32, CCDC24, Cep63, Cep70, CHCHD3, CRADD, CRSP9, CRX, CTDSP2, CTNNBIP1, DAZAP2, DDX6, DGCR6L, DHRS10, D1c2, DLX2, DVL2, ETV4, EWSR1, FANCG, FATE1, FLJ10726, FLJ22471, FLJ25439, FLJ32855, FTH1, FXR2, GADD45GIP1, GCC1, GOPC, HGS, HOXB9, HOXD3, KCTD1, KIAA0258, KIAA1683, KIAA1949, KIF9, KLC3, KLHL12, KRT15, KRT4, KRT6A, LENG1, LNX2, LOC283385, LOC388818, LOC391257, LOC595101, LOC91661, LZTS2, MAD1L1, MAD2L2, MEIS2, MGC11257, MGC27019, MGC4266, MINA, MKRN3, MLLT11, MLX, MRPS6, MYEF2, MYST2, NEFL, NIF3L1, NKAP, NOS3, NUDT18, OTX2, PCBP1, PIAS2, POLM, POPS, PSMA1, PSMF1, RBPMS, RFX4, RIBC2, RNPS1, SLC2A4, SORBS3, SOX5, SP100, SPAG5, SPG21, SSX21P, TEX11, TFIP11, TRIP6, TSC22D4, TTC23, UBE2I, VPS28, WDFY3, ZBTB16, ZCCHC7, ZMAT1, ZNF581, ZNF614, ZNHIT4 or a functional fragment or variant of any one thereof.

Complexes as taught herein involve the presently disclosed host target proteins which interact with one or more HIV virus proteins and/or with one or more HTLV virus proteins and thus participate in HIV biology and/or HTLV biology, respectively. Such complexes may be advantageously employed in various applications, such as inter alia in therapeutic, diagnostic and compound-screening applications.

Based on the herein realised interactions of host target proteins with specific HIV types (i.e., HIV-1 and/or HIV-2) as summarised in Table 1 above, Table 2 discloses further embodiments of the complexes, denoted as embodiments ‘(i)’ to ‘(vi)’.

TABLE 2
Embodiments (i) to (vi) of complexes as taught herein comprising, consisting substantially of
or consisting of a first and second proteins, wherein:

| Emb. | -the first protein is: | -the second protein is selected from: |
|---|---|---|
| (i) | a protein of HIV-1 or a functional | BACH1, C8orf56, CDC23, CRX, DIPA, DLAT, |
| | fragment or variant of an HIV-1 | DLX2, HOXA3, HOXD3, HSFY1, KCTD1, KLHL12, |
| | protein | L3MBTL3, LDOC1, LNX2, LOC541468, MIZF, |
| | | PDE9A, SF3A3, SFRS11, SS18L1, TRAF2 or a |
| | | functional fragment or variant of any one thereof; or |
| (ii) | a protein of HIV-2 or a functional | BACH1, DIPA, DLAT, DLX2, FKBP7, FLJ20097, |
| | fragment or variant of an HIV-2 | HOXA3, HOXD3, HSFY1, KCTD1, KLHL12, |
| | protein | KRT6A, L3MBTL3, LDOC1, LNX2, LOC541468, |
| | | MIZF, PDE9A, PLEKHF2, SF3A3, SS18L1, TRAF2 |
| | | or a functional fragment or variant of any one thereof; |
| | | or |
| (iii) | a protein of HIV-1 or a functional | BACH1, DIPA, DLAT, DLX2, HOXA3, HOXD3, |
| | fragment or variant of an HIV-1 | HSFY1, KCTD1, KLHL12, L3MBTL3, LDOC1, |
| | protein | LNX2, LOC541468, MIZF, PDE9A, SF3A3, SS18L1, |
| | | TRAF2 or a functional fragment or variant of any one |
| | | thereof; or |
| (iv) | a protein of HIV-1 or a functional | C8orf56, CDC23, CRX, SFRS11 or a functional |
| | fragment or variant of an HIV-1 | fragment or variant of any one thereof; or |
| | protein | |
| (v) | a protein of HIV-2 or a functional | BACH1, DIPA, DLAT, DLX2, HOXA3, HOXD3, |
| | fragment or variant of an HIV-2 | HSFY1, KCTD1, KLHL12, L3MBTL3, LDOC1, |
| | protein | LNX2, LOC541468, MIZF, PDE9A, SF3A3, SS18L1, |
| | | TRAF2 or a functional fragment or variant of any one |
| | | thereof; or |
| (vi) | a protein of HIV-2 or a functional | FKBP7, FLJ20097, KRT6A, PLEKHF2 or a functional |
| | fragment or variant of an HIV-2 | fragment or variant of any one thereof |
| | protein | |

Embodiments (i) and (ii) specify host proteins interacting with (i.e., ‘interactors’ or ‘host interactors’) one or more HIV-1 proteins or one or more HIV-2 proteins. These interactors provide valuable host targets for modulating the biology and/or pathogenicity of HIV-1 or HIV-2, respectively. Embodiments (iii) and (v) specify host interactors shared between HIV-1 and HIV-2, i.e., host proteins interacting with one or more HIV-1 proteins as well as with one or more HIV-2 proteins. Such interactors represent common host targets for modulating the biology and/or pathogenicity of both HIV-1 and HIV-2. Further, embodiment (iv) specifies HIV-1 interactors, which do not display an interaction with HIV-2 proteins; and embodiment (vi) specifies HIV-2 interactors which do not display an interaction with HIV-1 proteins. Such interactors provide host targets for selectively modulating the biology and/or pathogenicity of HIV-1 or HIV-2, respectively.

Based on the herein realised interactions of host target proteins with specific HIV-1 and/or HIV-2 proteins as summarised in Table 1 above, Table 3 discloses further embodiments of the complexes, denoted as embodiments ‘(vii)’ to ‘(xvi)’.

TABLE 3
Embodiments (vii) to (xvi) of complexes as taught herein comprising, consisting substantially
of or consisting of a first and second proteins, wherein:

| Emb. | - the first protein is: | -the second protein is selected from: |
|---|---|---|
| (vii) | HIV-1 gagpol or a functional | DIPA, HSFY1, SS18L1 or a functional fragment or |
| | fragment or variant thereof | variant of any one thereof; or |
| (viii) | HIV-1 gag or a functional fragment | DLAT, DLX2, HSFY1, KCTD1, KLHL12, LNX2, |
| | or variant thereof | TRAF2 or a functional fragment or variant of any one |
| | | thereof; or |
| (ix) | HIV-1 tat or a functional fragment | BACH1, HOXA3, HOXD3, L3MBTL3, LDOC1, |
| | or variant thereof | LNX2, LOC541468, MIZF, PDE9A, SF3A3 or a |
| | | functional fragment or variant of any one thereof; or |
| (x) | HIV-1 rev or a functional fragment | MIZF or a functional fragment or variant thereof; or |
| | or variant thereof | |
| (xi) | HIV-1 env or a functional fragment | HSFY1, SFRS11 or a functional fragment or variant of |
| | or variant thereof | any one thereof; or |
| (xii) | HIV-1 nef or a functional fragment | C8orf56, CDC23, CRX, HSFY1, LNX2 or a functional |
| | or variant thereof | fragment or variant of any one thereof; or |
| (xiii) | HIV-2 gagpol or a functional | DIPA, SS18L1, HSFY1 or a functional fragment or |
| | fragment or variant thereof | variant of any one thereof; or |
| (xiv) | HIV-2 gag or a functional fragment | DLAT, DLX2, HSFY1, KCTD1, KLHL12, LNX2, |
| | or variant thereof | SS18L1, TRAF2 or a functional fragment or variant of |
| | | any one thereof; or |
| (xv) | HIV-2 tat or a functional fragment | BACH1, FKBP7, FLJ20097, HOXA3, HOXD3, |
| | or variant thereof | KRT6A, L3MBTL3, LDOC1, LNX2, LOC541468, |
| | | MIZF, PDE9A, PLEKHF2, SF3A3 or a functional |
| | | fragment or variant of any one thereof; or |
| (xvi) | HIV-2 rev or a functional fragment | MIZF or a functional fragment or variant thereof |
| | or variant thereof | |

Based on the herein realised interactions of host target proteins with specific HTLV types (i.e., HTLV-1 and/or HTLV-2) as summarised in Table 1 above, Table 4 discloses further embodiments of the complexes, denoted as embodiments ‘(xvii)’ to ‘(xxii)’.

TABLE 4
Embodiments (xvii) to (xxii) of complexes as taught herein comprising, consisting
substantially of or consisting of a first and second proteins, wherein:

| Emb. | - the first protein is: | -the second protein is selected from: |
|---|---|---|
| (xvii) | a protein of HTLV-1 or a functional | BHLHB2, C10orf3, C16orf33, C1orf59, C20orf141, |
| | fragment or variant of an HTLV-1 | C20orf81, CCDC24, Cep63, CHCHD3, CRADD, |
| | protein | CRX, CTDSP2, CTNNBIP1, DAZAP2, DGCR6L, |
| | | DHRS10, Dlc2, DVL2, ETV4, EWSR1, FANCG, |
| | | FATE1, FLJ22471, FLJ25439, FLJ32855, FTH1, |
| | | GOPC, HGS, HOXB9, HOXD3, KIAA0258, |
| | | KIAA1949, KIF9, KRT15, KRT4, KRT6A, LENG1, |
| | | LNX2, LOC283385, LOC388818, LOC391257, |
| | | LOC595101, LOC91661, LZTS2, MAD2L2, MINA, |
| | | MLLT11, MLX, MRPS6, MYEF2, MYST2, NEFL, |
| | | NIF3L1, NOS3, NUDT18, OTX2, PCBP1, PIAS2, |
| | | POLM, POP5, PSMA1, RFX4, RIBC2, RNPS1, |
| | | SP100, SPAG5, SPG21, TRIP6, TSC22D4, TTC23, |
| | | UBE2I, VPS28, WDFY3, ZCCHC7, ZMAT1, |
| | | ZNF581, ZNF614, ZNHIT4 or a functional fragment or |
| | | variant of any one thereof; or |
| (xviii) | a protein of HTLV-2 or a functional | AP4M1, ARL6IP4, BHLHB2, C1orf94, C8orf32, |
| | fragment or variant of an HTLV-2 | CCDC24, CDC23, Cep63, Cep70, CRSP9, CRX, |
| | protein | DDX6, DGCR6L, Dlc2, DLX2, EWSR1, FATE1, |
| | | FLJ10726, FLJ22471, FLJ32855, FXR2, |
| | | GADD45GIP1, GCC1, HGS, KCTD1, KIAA1683, |
| | | KLC3, KLHL12, KRT15, LNX2, LOC388818, |
| | | LOC595101, LZTS2, MAD1L1, MEIS2, MGC11257, |
| | | MGC27019, MGC4266, MINA, MKRN3, MRPS6, |
| | | NIF3L1, NKAP, NUDT18, NUP62, POLM, PSMF1, |
| | | RBPMS, SLC2A4, SORBS3, SOX5, SP100, SPG21, |
| | | SSX2IP, TEX11, TFIP11, TRAF2, VPS28, ZBTB16, |
| | | ZNF581, ZNF614 or a functional fragment or variant |
| | | of any one thereof; or |
| (xix) | a protein of HTLV-1 or a functional | BHLHB2, CCDC24, Cep63, CRX, DGCR6L, Dlc2, |
| | fragment or variant of an HTLV-1 | EWSR1, FATE1, FLJ22471, FLJ32855, HGS, KRT15, |
| | protein | LNX2, LOC388818, LOC595101, LZTS2, MINA, |
| | | MRPS6, NIF3L1, NUDT18, POLM, SP100, SPG21, |
| | | VPS28, ZNF581, ZNF614, or a functional fragment or |
| | | variant of any one thereof; or |
| (xx) | a protein of HTLV-1 or a functional | C10orf3, C16orf33, C1orf59, C20orf141, C20orf81, |
| | fragment or variant of an HTLV-1 | CHCHD3, CRADD, CTDSP2, CTNNBIP1, DAZAP2, |
| | protein | DHRS10, DVL2, ETV4, FANCG, FLJ25439, FTH1, |
| | | GOPC, HOXB9, HOXD3, KIF9, KIAA0258, |
| | | KIAA1949, KRT4, KRT6A, LENG1, LOC283385, |
| | | LOC391257, LOC91661, MAD2L2, MLLT11, MLX, |
| | | MYEF2, MYST2, NEFL, NOS3, OTX2, PCBP1, |
| | | PIAS2, POP5, PSMA1, RFX4, RIBC2, RNPS1, |
| | | SPAG5, TRIP6, TSC22D4, TTC23, UBE2I, WDFY3, |
| | | ZCCHC7, ZMAT1, ZNHIT4 or a functional fragment |
| | | or variant of any one thereof; or |
| (xxi) | a protein of HTLV-2 or a functional | BHLHB2, CCDC24, CDC23, Cep63, CRX, DGCR6L, |
| | fragment or variant of an HTLV-2 | Dlc2, EWSR1, FATE1, FLJ22471, FLJ32855, HGS, |
| | protein | KRT15, LNX2, LOC388818, LOC595101, LZTS2, |
| | | MINA, MRPS6, NIF3L1, NUDT18, NUP62, POLM, |
| | | SP100, SPG21, VPS28, ZNF581, ZNF614 or a |
| | | functional fragment or variant of any one thereof; or |
| (xxii) | a protein of HTLV-2 or a functional | AP4M1, ARL6IP4, C1or194, C8orf32, Cep70, CRSP9, |
| | fragment or variant of an HTLV-2 | DDX6, DLX2, FLJ10726, FXR2, GADD45GIP1, |
| | protein | GCC1, KCTD1, KIAA1683, KLC3, KLHL12, |
| | | MAD1L1, MEIS2, MGC11257, MGC27019, |
| | | MGC4266, MKRN3, NKAP, PSMF1, RBPMS, |
| | | SLC2A4, SORBS3, SOX5, SSX2IP, TEX11, TFIP11, |
| | | TRAF2, ZBTB16 or a functional fragment or variant of |
| | | any one thereof |

Embodiments (xvii) and (xviii) specify host interactors of one or more HTLV-1 proteins or one or more HTLV-2 proteins. These interactors provide valuable host targets for modulating the biology and/or pathogenicity of HTLV-1 or HTLV-2, respectively. Embodiments (xix) and (xxi) specify host interactors shared between HTLV-1 and HTLV-2, i.e., host proteins interacting with one or more HTLV-1 proteins as well as with one or more HTLV-2 proteins. Such interactors represent common host targets for modulating the biology and/or pathogenicity of both HTLV-1 and HTLV-2. Further, embodiment (xx) specifies HTLV-1 interactors which do not display an interaction with HTLV-2 proteins; and embodiment (xxii) specifies HTLV-2 interactors which do not display an interaction with HTLV-1 proteins. Such interactors provide host targets for selectively modulating the biology and/or pathogenicity of HTLV-1 or HTLV-2, respectively.

Based on the herein realised interactions of host target proteins with specific HTLV-1 and/or HTLV-2 proteins as summarised in Table 1 above, Table 5 discloses further embodiments of the complexes, denoted as embodiments ‘(xxiii)’ to ‘(xxxi)’.

TABLE 5
Embodiments (xxiii) to (xxxi) of complexes as taught herein comprising, consisting
substantially of or consisting of a first and second proteins, wherein:

| Emb. | - the first protein is: | -the second protein is selected from: |
|---|---|---|
| (xxiii) | HTLV-1 rex or a functional | BHLHB2, C10orf3, CRX, Dlc2, DVL2, FLJ25439, |
| | fragment or variant thereof | LNX2, LOC391257, LZTS2, NIF3L1, NUP62, PIAS2, |
| | | RIBC2, SP100, SPAG5, SPG21, UBE2I, ZCCHC7 or a |
| | | functional fragment or variant of any one thereof; or |
| (xxiv) | HTLV-1 tax or a functional | C16orf33, C1orf59, C20orf141, CCDC24, Cep63, |
| | fragment or variant thereof | CHCHD3, CRADD, CRX, CTDSP2, CTNNBIP1, |
| | | DGCR6L, DHRS10, ETV4, EWSR1, FANCG, |
| | | FATE1, FLJ22471, FLJ25439, FLJ32855, HGS, |
| | | HOXB9, KIAA0258, KIAA1949, KIF9, KRT15, |
| | | KRT4, KRT6A, LENG1, LNX2, LOC283385, |
| | | LOC388818, LOC595101, LOC91661, MAD2L2, |
| | | MINA, MRPS6, MYEF2, NEFL, NIF3L1, NOS3, |
| | | NUDT18, POLM, POP5, PSMA1, RFX4, RIBC2, |
| | | SPG21, TRIP6, TTC23, VPS28, WDFY3, ZMAT1, |
| | | ZNF581, ZNF614, ZNHIT4 or a functional fragment or |
| | | variant of any one thereof; or |
| (xxv) | HTLV-1 env or a functional | TSC22D4, C20orf81 or a functional fragment or |
| | fragment or variant thereof | variant of any one thereof; or |
| (xxvi) | HTLV-1 hbz or a functional | DAZAP2, FTH1, GOPC, HOXD3, MLLT11, MLX, |
| | fragment or variant thereof | MYST2, OTX2, PCBP1, RNPS1 or a functional |
| | | fragment or variant of any one thereof; or |
| (xxvii) | HTLV-2 gag or a functional | DLX2, KCTD1, KLHL12, LNX2, TFIP11, TRAF2 or |
| | fragment or variant thereof | a functional fragment or variant of any one thereof; or |
| (xxviii) | HTLV-2 pol or a functional | KLC3, KRT15, MEIS2, MKRN3, RBPMS, SORBS3, |
| | fragment or variant thereof | TEX11, TFIP11 or a functional fragment or variant of |
| | | any one thereof; or |
| (xxix) | HTLV-2 rex or a functional | CRX, Dlc2, LNX2, NIF3L1, NUP62, SP100, SPG21 or |
| | fragment or variant thereof | a functional fragment or variant of any one thereof; or |
| (xxx) | HTLV-2 tax2 or a functional | AP4M1, ARL6IP4, BHLHB2, C1orf94, C8orf32, |
| | fragment or variant thereof | CCDC24, CDC23, Cep63, Cep70, CRX, DDX6, |
| | | DGCR6L, Dlc2, EWSR1, FATE1, FLJ10726, |
| | | FLJ22471, FLJ32855, FXR2, GADD45GIP1, GCC1, |
| | | HGS, KIAA1683, KRT15, LNX2, LOC388818, |
| | | LOC595101, LZTS2, MAD1L1, MGC11257, |
| | | MGC27019, MGC4266, MINA, MRPS6, NIF3L1, |
| | | NKAP, NUDT18, NUP62, POLM, PSMF1, SLC2A4, |
| | | SOX5, SP100, SPG21, SSX2IP, VPS28, ZBTB16, |
| | | ZNF581, ZNF614 or a functional fragment or variant |
| | | of any one thereof; or |
| (xxxi) | HTLV-2 env or a functional | CRSP9 or a functional fragment or variant thereof |
| | fragment or variant thereof | |

Based on the herein realised interactions of host target proteins with HIV and/or HTLV proteins as summarised in Table 1 above, Table 6 discloses further embodiments of the complexes, denoted as embodiments ‘(xxxii)’ to ‘(xxxv)’.

TABLE 6
Embodiments (xxxii) to (xxxv) of complexes as taught herein comprising, consisting
substantially of or consisting of a first and second proteins, wherein:

| Emb. | - the first protein is: | -the second protein is selected from: |
|---|---|---|
| (xxxii) | a protein of HIV or a functional | CDC23, CRX, DLX2, HOXD3, KCTD1, KLHL12, |
| | fragment or variant of an HIV | KRT6A, LNX2, TRAF2 or a functional fragment or |
| | protein | variant of any one thereof; or |
| (xxxiii) | a protein of HIV or a functional | BACH1, C8orf56, DIPA, DLAT, FKBP7, FLJ20097, |
| | fragment or variant of an HIV | HOXA3, HSFY1, L3MBTL3, LDOC1, LOC541468, |
| | protein | MIZF, PDE9A, PLEKHF2, SF3A3, SFRS11, |
| | | SS18L1 or a functional fragment or variant of any |
| | | one thereof; or |
| (xxxiv) | a protein of HTLV or a functional | CRX, DLX2, HOXD3, KCTD1, KLHL12, KRT6A, |
| | fragment or variant of an HTLV | LNX2, TRAF2 or a functional fragment or variant of |
| | protein | any one thereof; or |
| (xxxv) | a protein of HTLV or a functional | AP4M1, ARL6IP4, BHLHB2, C10orf3, C16orf33, |
| | fragment or variant of an HTLV | C1orf59, C1orf94, C20orf141, C20orf81, C8orf32, |
| | protein | CCDC24, Cep63, Cep70, CHCHD3, CRADD, |
| | | CRSP9, CTDSP2, CTNNBIP1, DAZAP2, DDX6, |
| | | DGCR6L, DHRS10, Dlc2, DVL2, ETV4, EWSR1, |
| | | FANCG, FATE1, FLJ10726, FLJ22471, FLJ25439, |
| | | FLJ32855, FTH1, FXR2, GADD45GIP1, GCC1, |
| | | GOPC, HGS, HOXB9, KIAA0258, KIAA1683, |
| | | KIAA1949, KIF9, KLC3, KRT15, KRT4, LENG1, |
| | | LOC283385, LOC388818, LOC391257, |
| | | LOC595101, LOC91661, LZTS2, MAD2L2, |
| | | MEIS2, MGC11257, MGC27019, MGC4266, |
| | | MINA, MKRN3, MLLT11, MLX, MRPS6, MYEF2, |
| | | MYST2, NEFL, NIF3L1, NKAP, NOS3, NUDT18, |
| | | OTX2, PCBP1, PIAS2, POLM, POP5, PSMA1, |
| | | PSMF1, RBPMS, RFX4, RIBC2, RNPS1, SLC2A4, |
| | | SORBS3, SOX5, SP100, SPAG5, SPG21, SSX2IP, |
| | | TEX11, TFIP11, TRIP6, TSC22D4, TTC23, UBE2I, |
| | | VPS28, WDFY3, ZBTB16, ZCCHC7, ZMAT1, |
| | | ZNF581, ZNF614, ZNHIT4 or a functional fragment |
| | | or variant of any one thereof |

Embodiments (xxxii) and (xxxiv) specify host interactors shared between HIV and HTLV viruses, i.e., host proteins interacting with one or more HIV proteins as well as with one or more HTLV proteins. Such interactors thus represent common host targets highly relevant for modulating retroviral biology and/or pathogenicity in general, including HIV and HTLV biology and/or pathogenicity. Further, embodiment (xxxiii) specifies HIV interactors which do not display an interaction with HTLV proteins; and embodiment (xxxv) specifies HTLV interactors which do not display an interaction with HIV proteins. Such interactors provide host targets for selectively modulating the biology and/or pathogenicity of HIV or HTLV, respectively.

The Applicant has further performed functional analyses to evaluate the roles of the herein identified host interactors in retroviral biology and/or pathogenicity, and delineated subgroups of the host interactors which are involved in various aspects of the biology and/or pathogenicity of HIV and/or HTLV and which constitute preferred targets in diseases and conditions caused by HIV and/or HTLV, respectively.

For example, many of the herein identified host interactor proteins can modulate transactivation of HIV viral promoter sequences by HIV Tat proteins and/or transactivation of HTLV viral promoter sequences by HTLV Tax proteins. The Applicant particularly contemplates that host proteins which enhance Tat or Tax transactivation activity may play important roles in viral replication and persistence in infected cells; and host proteins which reduce Tat or Tax transactivation activity may be implicated in viral latency allowing HIV or HTLV viruses to escape immune surveillance, or in coordinating distinct phases of the viruses cycles. Particularly inhibition of said host proteins and/or complexes in which they participate may be therapeutically advantageous in retroviral diseases and conditions

Consequently, Tables 7 and 8 disclose further preferred embodiments of complexes taught herein, denoted as embodiments (xxxvi) to (liii), wherein host interactor proteins participating in said complexes can affect HIV Tat and/or HTLV Tax transactivation. In said tables, “Tat” and “Tax” symbolise, respectively, transactivation of HIV viral promoter sequences by HIV Tat or transactivation of HTLV viral promoter sequences by HTLV Tax; “Y” and “N” denote, respectively, that a given host interactor can modulate or does not modulate the transactivation; “+” and “−” denote, respectively, that a given host interactor can enhance or reduce the transactivation.

TABLE 7
Embodiments (xxxvi) to (xlii) of complexes as taught herein comprising,
consisting substantially of or consisting of a first and second proteins,
wherein the first protein is an HIV protein or a functional fragment or
variant of an HIV protein, optionally as defined in any one of
embodiments (i) to (xvi) taught herein, and wherein:

| Emb. | -the second protein is selected from: | Tat | Tax |
|---|---|---|---|
| (xxxvi) | DLX2, HOXA3, LNX2 or a functional fragment | Y | Y |
| | or variant of any one thereof; or | | |
| (xxxvii) | CRX, DLAT, HOXD3, LOC541468, MIZF, | Y | N |
| | PDE9A, SF3A3, TRAF2 or a functional fragment | | |
| | or variant of any one thereof; or | | |
| (xxxviii) | DIPA, HSFY1, KCTD1 or a functional fragment | N | Y |
| | or variant of any one thereof; or | | |
| (xxxix) | CRX, PDE9A, SF3A3, TRAF2 or a functional | − | N |
| | fragment or variant of any one thereof; or | | |
| (xl) | DLX2, HOXA3, LNX2 or a functional fragment | − | − |
| | or variant of any one thereof; or | | |
| (xli) | DIPA, HSFY1, KCTD1 or a functional fragment | N | − |
| | or variant of any one thereof; or | | |
| (xlii) | DLAT, HOXD3, LOC541468, MIZF or a | + | N |
| | functional fragment or variant of any one thereof | | |

TABLE 8
Embodiments (xliii) to (liii) of complexes as taught herein comprising, consisting
substantially of or consisting of a first and second proteins, wherein the first protein
is an HTLV protein or a functional fragment or variant of an HTLV protein, optionally
as defined in any one of embodiments (xvii) to (xxxi) taught herein, and wherein:

| Emb. | -the second protein is selected from: | Tat | Tax |
|---|---|---|---|
| (xliii) | C10orf3, C20orf141, DLX2, FANCG, GCC1, KRT8, LNX2, | Y | Y |
| | LOC391257, LZTS2, TFIP11, TSC22D4, ZNF614 or a functional | | |
| | fragment or variant of any one thereof; or | | |
| (xliv) | BHLHB2, C1orf94, C8orf32, CCDC24, CRSP9, DAZAP2, DGCR6L, | N | Y |
| | DHRS10, FATE1, FLJ10726, FLJ22471, FTH1, FXR2, | | |
| | GADD45GIP1, GOPC, HOXB9, KCTD1, KIAA1949, KIF9, KLC3, | | |
| | LENG1, MAD1L1, MRPS6, MYEF2, MYST2, NEFL, NIF3L1, | | |
| | NOS3, OTX2, PCBP1, PSMF1, RNPS1, SPG21, TTC23 or a | | |
| | functional fragment or variant of any one thereof; or | | |
| (xlv) | C16orf33, CRX, DVL2, HOXD3, KRT15, LOC91661, MINA, | Y | N |
| | MLLT11, NKAP, PIAS2, SP100, SPAG5, TEX11, TRAF2, TRIP6, | | |
| | UBE2I, ZMAT1 or a functional fragment or variant of any one | | |
| | thereof; or | | |
| (xlvi) | BHLHB2, C1orf94, C8orf32, DGCR6L, DHRS10, FATE1, FLJ10726, | N | − |
| | FTH1, FXR2, GADD45GIP1, KCTD1, KIAA1949, KIF9, KLC3, | | |
| | LENG1, MAD1L1, MRPS6, NEFL, NIF3L1, NOS3, PCBP1, SPG21, | | |
| | TTC23 or a functional fragment or variant of any one thereof; or | | |
| (xlvii) | C10orf3, GCC1 or a functional fragment or variant of any one thereof; | + | − |
| | or | | |
| (xlviii) | DLX2, KRT8, LNX2, LOC391257, LZTS2, TFIP11 or a functional | − | − |
| | fragment or variant of any one thereof; or | | |
| (xlix) | CCDC24, CRSP9, DAZAP2, FLJ22471, GOPC, HOXB9, MYEF2, | N | + |
| | MYST2, OTX2, PSMF1, RNPS1 or a functional fragment or variant of | | |
| | any one thereof; or | | |
| (l) | TSC22D4 or a functional fragment or variant thereof; or | + | + |
| (li) | C20orf141, FANCG, ZNF614 or a functional fragment or variant of | − | + |
| | any one thereof; or | | |
| (lii) | CRX, DVL2, KRT15, LOC91661, MINA, PIAS2, SPAG5, TRAF2, | − | N |
| | ZMAT1 or a functional fragment or variant of any one thereof; or | | |
| (liii) | HOXD3, MLLT11, NKAP, SP100, TEX11, TRIP6, UBE2I or a | + | N |
| | functional fragment or variant of any one thereof | | |

Particularly preferred may be second proteins chosen from TSC22D4, HOXA3, LNX2, DLX2, LZTS2, LOC391257, KRT8, TFIP11, SPAG5, SF3A3, FLJ10726, MAD1L1, SPG21 (or a functional fragment or variant of any one thereof) which modulate transactivation of both HIV and HTLV LTR, even more preferred may be TSC22D4 which increases transactivation of both HIV and HTLV LTR.

In a further example, several of the herein identified host interactor proteins are directly or indirectly involved in cellular pathways, such as inter alia in metabolic or signalling pathways and/or in pathways connected to diseases. The Applicant particularly contemplates that host proteins which are co-associated with cellular pathways, such as preferably but without limitation with the Notch pathway, apoptosis pathway and/or ubiquitin mediated proteolysis pathway, may play central roles in biology and/or pathogenicity of retroviruses.

Consequently, Tables 9 and 10 disclose further preferred embodiments of complexes taught herein, denoted as embodiments (liv) to (lxxxii), wherein host interactor proteins included in said complexes are directly or indirectly (e.g., through communication or interaction with one or more cellular components such as proteins participating in a pathway) involved in the cellular pathways specified in said tables.

TABLE 9
Embodiments (liv) to (lxvii) of complexes as taught herein comprising, consisting
substantially of or consisting of a first and second proteins, wherein the first protein is an
HIV protein or a functional fragment or variant of an HIV protein, optionally as defined in
any one of embodiments (i) to (xvi) taught herein, and wherein:

| Emb. | -the second protein is selected from: | Pathway |
|---|---|---|
| (liv) | LNX2, SS18L1, TRAF2 or a functional fragment or variant of | Notch |
| | any one thereof; or | |
| (lv) | MIZF, TRAF2 or a functional fragment or variant of any one | Apoptosis |
| | thereof; or | |
| (lvi) | BACH1, CDC23, LNX2, PDE9A, TRAF2 or a functional | Ubiquitin mediated |
| | fragment or variant of any one thereof; or | proteolysis |
| (lvii) | DIPA, KLHL12, TRAF2 or a functional fragment or variant of | Chronic myeloid |
| | any one thereof; or | leukaemia |
| (lviii) | KLHL12, KRT6A, TRAF2 or a functional fragment or variant | Colorectal cancer |
| | of any one thereof; or | |
| (lix) | DIPA, KLHL12, KRT6A, TRAF2 or a functional fragment or | ErbB signalling |
| | variant of any one thereof; or | |
| (lx) | DIPA, KLHL12, KRT6A, TRAF2 or a functional fragment or | Glioma |
| | variant of any one thereof; or | |
| (lxi) | SS18L1, TRAF2 or a functional fragment or variant of any one | Huntington's disease |
| | thereof; or | |
| (lxii) | DIPA, KLHL12, TRAF2 or a functional fragment or variant of | Insulin signalling |
| | any one thereof; or | |
| (lxiii) | KLHL12, LDOC1, SS18L1 or a functional fragment or variant | Long-term potentiation |
| | of any one thereof; or | |
| (lxiv) | DIPA, TRAF2 or a functional fragment or variant of any one | Lysine degradation |
| | thereof; or | |
| (lxv) | DIPA, KLHL12, KRT6A or a functional fragment or variant of | Melanoma |
| | any one thereof; or | |
| (lxvi) | DIPA, KLHL12, KRT6A, LDOC1, SS18L1, TRAF2 or a | Prostate cancer |
| | functional fragment or variant of any one thereof; or | |
| (lxvii) | MIZF, TRAF2 or a functional fragment or variant of any one | Small cell lung cancer |
| | thereof | |

TABLE 10
Embodiments (lxviii) to (lxxxii) of complexes as taught herein comprising, consisting
substantially of or consisting of a first and second proteins, wherein the first protein is an HTLV
protein or a functional fragment or variant of an HTLV protein, optionally as defined in any one of
embodiments (xvii) to (xxxi) taught herein, and wherein:

| Emb. | -the second protein is selected from: | Pathway |
|---|---|---|
| (lxviii) | DVL2, EWSR1, FLJ32855, KLHL12, TRAF2 or a functional | Notch |
| | fragment or variant of any one thereof; or | |
| (lxix) | Dlc2, EWSR1, FATE1, FLJ32855, HGS, LZTS2, NIF3L1, | Apoptosis |
| | NUDT18, RIBC2, SPG21, TRAF2 or a functional fragment or | |
| | variant of any one thereof; or | |
| (lxx) | BHLHB2, CDC23, Dlc2, EWSR1, HGS, LNX2, NUDT18, | Ubiquitin mediated |
| | PIAS2, PSMA1, PSMF1, RIBC2, TRAF2, UBE2I or a | proteolysis |
| | functional fragment or variant of any one thereof; or | |
| (lxxi) | EWSR1, HGS, KLHL12, MINA, TRAF2 or a functional | Chronic myeloid |
| | fragment or variant of any one thereof; or | leukaemia |
| (lxxii) | DVL2, EWSR1, FLJ32855, HGS, KLHL12, KRT6A, MINA, | Colorectal cancer |
| | TRAF2 or a functional fragment or variant of any one thereof; | |
| | or | |
| (lxxiii) | EWSR1, HGS, KLHL12, KRT6A, LZTS2, MINA, TRAF2 or a | ErbB signalling |
| | functional fragment or variant of any one thereof; or | |
| (lxxiv) | EWSR1, HGS, KLHL12, KRT6A, TRAF2 or a functional | Glioma |
| | fragment or variant of any one thereof; or | |
| (lxxv) | EWSR1, HGS, TRAF2 or a functional fragment or variant of | Huntington's disease |
| | any one thereof; or | |
| (lxxvi) | EWSR1, HGS, KLHL12, SLC2A4, TRAF2 or a functional | Insulin signalling |
| | fragment or variant of any one thereof; or | |
| (lxxvii) | EWSR1, KLHL12 or a functional fragment or variant of any | Long-term potentiation |
| | one thereof; or | |
| (lxxviii) | BHLHB2, EWSR1, FLJ32855, HGS, TRAF2 or a functional | Lysine degradation |
| | fragment or variant of any one thereof; or | |
| (lxxix) | EWSR1, HGS, KLHL12, KRT6A or a functional fragment or | Melanoma |
| | variant of any one thereof; or | |
| (lxxx) | EWSR1, HGS, KLHL12, KRT6A, TRAF2 or a functional | Prostate cancer |
| | fragment or variant of any one thereof; or | |
| (lxxxi) | Dlc2, FATE1, FLJ32855, HGS, LZTS2, MINA, NIF3L1, | Small cell lung cancer |
| | NOS3, NUDT18, NUP62, PIAS2, RIBC2, SPG21, TRAF2 or a | |
| | functional fragment or variant of any one thereof; or | |
| (lxxxii) | EWSR1 or a functional fragment or variant thereof | Olfactory transduction |

Notably, the Applicant has identified the HIV- and HTLV-interactor TRAF2 (tumor necrosis factor TNF receptor-associated factor type 2) as a central node mediating interactions between HIV and HTLV proteins and relevant cellular pathways, including without limitation the Notch pathway, apoptosis pathway and ubiquitin mediated proteolysis pathway. Complexes comprising TRAF2 are thus particularly intended herein.

Also, the Applicant has identified the interactors LNX2, MIZF and TSC22D4 as potentially mediating interactions between HIV and HTLV proteins and relevant cellular pathways, including without limitation the Notch pathway, apoptosis pathway and/or ubiquitin mediated proteolysis pathway. Complexes comprising LNX2, MIZF and TSC22D4 are thus also particularly intended herein.

Accordingly, also disclosed herein are any one and all of the following:

(1) an agent that is able to modulate any one or more of the pathways identified in Table 10, preferably an agent that is able to modulate any one or more of Notch pathway, apoptosis pathway and ubiquitin mediated proteolysis pathway, even more preferably an agent that is able to modulate the Notch pathway, for use in the treatment of a disease or condition associated with a retrovirus, preferably wherein the disease or condition associated with a retrovirus is a disease or condition associated with a human retrovirus including human pathogenic and non-pathogenic retrovirus, more preferably HIV or HTLV.
(2) use of an agent that is able to modulate any one or more of the pathways identified in Table 10, preferably an agent that is able to modulate any one or more of Notch pathway, apoptosis pathway and ubiquitin mediated proteolysis pathway, even more preferably an agent that is able to modulate the Notch pathway, for the manufacture of a medicament for the treatment of a disease or condition associated with a retrovirus, preferably wherein the disease or condition associated with a retrovirus is a disease or condition associated with a human retrovirus including human pathogenic and non-pathogenic retrovirus, more preferably HIV or HTLV; or use of said agent for the treatment of said disease or condition;
(3) a method for treating a disease or condition associated with a retrovirus, preferably wherein the disease or condition associated with a retrovirus is a disease or condition associated with a human retrovirus including human pathogenic and non-pathogenic retrovirus, more preferably HIV or HTLV, in a subject in need of such treatment, comprising administering to said subject a therapeutically or prophylactically effective amount of an agent that is able to modulate any one or more of the pathways identified in Table 10, preferably an agent that is able to modulate any one or more of Notch pathway, apoptosis pathway and ubiquitin mediated proteolysis pathway, even more preferably an agent that is able to modulate the Notch pathway;
(4) The subject matter as set forth in any one of (1) to (3) above, wherein the agent is able to reduce (inhibit) or increase the activity of said one or more pathways;
(5) The subject matter as set forth in any one of (1) to (4) above, wherein said agent is able to specifically bind to one or more molecules (e.g., polypeptide or proteins, such as e.g., transcription factors, receptors, etc.) involved in said one or more pathways;
(6) The subject matter as set forth in any one of (1) to (5) above, wherein said agent is an antibody or a fragment or derivative thereof; a polypeptide; a peptide; a peptidomimetic; an aptamer; a photoaptamer; or a chemical substance, preferably an organic molecule, more preferably a small organic molecule;
(7) The subject matter as set forth in any one of (1) to (4) above, wherein the agent is able to reduce or inhibit the expression of one or more molecules (e.g., polypeptide or proteins, such as e.g., transcription factors, receptors, etc.) involved in said one or more pathways, preferably wherein said agent is an antisense agent; a ribozyme; or an agent capable of causing RNA interference;
(8) The subject matter as set forth in any one of (1) to (4) above, wherein said agent is able to reduce or inhibit the level and/or activity of one or more molecules (e.g., polypeptide or proteins, such as e.g., transcription factors, receptors, etc.) involved in said one or more pathways, preferably wherein said agent is a recombinant or isolated deletion construct of the said one or more molecules having a dominant negative activity over the native one or more molecules;
(9) An assay to select, from a group of test agents, a candidate agent potentially useful in the treatment of a disease or condition associated with a retrovirus, preferably wherein the disease or condition associated with a retrovirus is a disease or condition associated with a human retrovirus including human pathogenic and non-pathogenic retrovirus, more preferably HIV or HTLV, said assay comprising determining whether a tested agent can modulate any one or more of the pathways identified in Table 10, preferably any one or more of Notch pathway, apoptosis pathway and ubiquitin mediated proteolysis pathway, even more preferably the Notch pathway;
(10) The assay as set forth in (9) above, further comprising use of the selected candidate agent for the preparation of a composition for administration to and monitoring the prophylactic and/or therapeutic effect thereof in a non-human animal model, preferably a non-human mammal model, of any one disease or condition as defined in (9) above.

As demonstrated in example 18, the Applicant has realised that the Notch pathway may be centrally involved in retroviral infection, and in particular that inhibition of the Notch pathway significantly lowers retroviral infection. Consequently, modulation and preferably inhibition of Notch pathway is particularly intended herein. In this respect, Notch pathway inhibitors may be applied in counteracting retroviral infections. A “Notch inhibitor” generally refers to any agent capable of blocking Notch signaling. Mechanisms of action of such NOTCH inhibitors include, but are not limited to, inhibition of gamma-secretase and subsequent suppression of Notch receptor cleavage, inhibition of Notch trafficking to the cell membrane, suppression of expression or function of ligands and/or receptors, inhibition of ligand turnover, cleavage, and/or endocytosis, modification of Notch glycosylation, alteration of ubiquitination of Notch components including the Notch intracellular domain, modification of expression and/or activity of co-factors or effectors (e.g., members of the MAML family, RBP-Jkappa/CBF-1), and alteration of differentiation/population of undifferentiated cells in bone marrow or circulating blood. Preferred inhibitors include receptor antagonists that block the binding of Notch ligands to receptors, RNA interfering agents for Notch components, blocking antibodies against Notch components, and, most preferably, gamma-secretase inhibitors. An alternative approach would be a systemic or local delivery of a nucleic acid plasmid encoding a Notch component or a dominant negative form of such a component.

As used herein, the term “gamma secretase inhibitor” is any compound with the ability to inhibit the activity of gamma-secretase. Commonly, gamma secretase inhibitors may be short peptides (e.g., about two to about five amino acid residues) comprised primarily of hydrophobic amino acids or peptidomimetic agents that structurally resemble such peptides. Non-limiting examples of gamma secretase inhibitors include the tripeptide aldehyde N-benzyloxycarbonyl-leucyl-leucyl-norleucinal (z-Leu-Leu-Nle-CHO), L-685,458 (Shearman et al. Biochemistry, 2000, vol. 39, 8698-8704); LY411,575 (Wong et al. J Biol Chem, 2004, vol. 279, 12876-82); a cell-permeable (hydroxyethyl)urea peptidomimetic WPE-III-31C (Campbell et al. Biochemistry, 2002, vol. 41, 3372; Esler et al. Proc. Natl. Acad. Sci. USA, 2002, vol. 99, 2720; Kimberly et al. J. Biol. Chem., 2002, vol. 277, 35113); a benzodiazepinyl-γ-hydroxybutyramide compound XIX (2S,3R)-3-(3,4-Difluorophenyl)-2-(4-fluorophenyl)-4-hydroxy-N-((3S)-2-oxo-5-phenyl-2,3-dihydro-1H-benzo[e][1,4]diazepin-3-yl)-butyramide compound XVII (Churcher et al. J. Med. Chem., 2003, vol. 46, 2275) and LY-374973, N—[N-(3,5-Difluorophenacetyl)-L-alanyl]-S-phenylglycine t-butyl ester (DAPT) (Micchelli et al., FASEB J., 2003, vol. 17, 78-81).

The Applicant has also realised a number of further aspects and embodiments of the invention.

Hence, also encompassed herein is an isolated nucleic acid encoding any complex as taught herein. Preferably, the nucleic acid may encode at least the first and second proteins of the complex. The first and second proteins of the complex may be encoded by the same molecule of said nucleic acid (i.e., in cis), or the first and second proteins of the complex may be encoded by separate or distinct molecules of said nucleic acid (i.e., in trans). Preferably, the nucleic acid and in particular the sequences thereof encoding the first and second proteins of the complex may be operably linked to one or more regulatory sequences allowing for expression of the nucleic acid. For instance, regulatory sequences as intended herein may allow for expression of nucleic acids in vitro (e.g., in a cell-free expression system), in a host cell, host organ and/or host organism.

Further disclosed is a vector comprising the nucleic acid as taught herein. Preferably, the vector may be an expression vector, wherein the nucleic acid and in particular the sequences thereof encoding the first and second proteins of the complex is operably linked to one or more regulatory sequences allowing for expression of the nucleic acid. Where the first and second proteins of the complex are encoded by separate or distinct molecules of the nucleic acid, said nucleic acid molecules may be comprised in the same vector or in separate or distinct vectors (i.e., in trans). Also contemplated is a method for producing the above vector, comprising introducing the nucleic acid as taught herein to a recipient vector.

Further disclosed is a host cell comprising any isolated complex, isolated nucleic acid or vector as taught herein; and a method for producing such host cell comprising introducing said isolated complex, isolated nucleic acid or vector to a recipient host cell. Preferably, the host cell may be a prokaryotic or eukaryotic cell, more preferably a bacterial, fungal, plant or animal cell, even more preferably a mammal cell or a primate cell, including human cells, non-human mammal cells and non-human primate cells. The isolated nucleic acid or vector may be integrated, preferably stably integrated, into the genome of the host cell or may remain extra-genomic or extra-chromosomal. Insofar the host cell comprises said isolated nucleic acid or vector, it may be denoted a ‘transgenic’ or ‘transformed’ cell in that regard. Preferably, the host cell expresses or is under suitable conditions capable of expressing the isolated nucleic acid or vector comprised therein, thus producing the encoded complex.

Also disclosed is a host organism comprising any isolated complex, isolated nucleic acid, vector or host cell as taught herein; and a method for producing such host organism comprising introducing said isolated complex, isolated nucleic acid or vector to a recipient host organism, e.g., to a cell, tissue or organ of said host organism, or introducing said host cell to a recipient host organism, or at least partly regenerating an organism from said host cell. Preferably, the host organism may be a multi-cellular organism, more preferably a plant or animal organism, even more preferably a mammal or primate, particularly including non-human mammals and non-human primates. The isolated nucleic acid or vector may be integrated, preferably stably integrated, into the genome of the host organism or may remain extra-genomic or extra-chromosomal. Insofar the host organism comprises said isolated nucleic acid or vector, it may be denoted a ‘transgenic’ or ‘transformed’ organism in that regard. Preferably, the host organism expresses or is under suitable conditions capable of expressing the isolated nucleic acid or vector comprised therein, hence producing the encoded complex.

As well encompassed is a progeny of the host cell or host organism as taught herein. Particularly intended is progeny comprising the introduced isolated complex, isolated nucleic acid or vector, or comprising a replicated copy of said nucleic acid or vector, i.e., progeny transgenic or transformed with regard to said nucleic acid or vector.

Further contemplated are methods for producing any isolated complex as taught herein, comprising: expressing the isolated nucleic acid or vector as taught herein in an in vitro reaction (e.g., in a cell-free expression system), thereby producing said complex, and optionally and preferably at least partly purifying the complex from said reaction; or culturing or maintaining the host cell or host organism as taught herein under conditions conducive to expression of the nucleic acid or vector as taught herein in the host cell or host organism, thereby producing said complex, and optionally and preferably at least partly purifying the complex from said host cell or host organism; or providing constituents of the complex comprising, consisting substantially of or consisting of the first and second proteins of the complex, contacting said constituents under conditions conducive to interaction there between, thereby producing said complex, and optionally and preferably at least partly purifying the complex from said constituents; or providing a biological same comprising said complex and at least partly purifying the complex from said biological sample.

Also intended are compositions and formulations comprising any isolated complex, isolated nucleic acid, vector, host cell or host organism as taught herein, and one or more additional components, such as without limitation one or more solvents and/or one or more pharmaceutically acceptable carriers. Further provided are methods for producing the above compositions or formulations, comprising admixing the isolated complex, isolated nucleic acid, vector, host cell or host organism as taught herein with one or more additional components.

Particularly intended are pharmaceutical compositions and formulations comprising any isolated complex, isolated nucleic acid, vector, host cell or host organism as taught herein and one or more pharmaceutically acceptable carriers; and methods for producing said pharmaceutical compositions and formulations, comprising admixing the isolated complex, isolated nucleic acid, vector or host cell as taught herein with said one or more pharmaceutically acceptable carriers.

Further disclosed herein are kits of parts comprising any one or more isolated complex (or optionally unbound constituents of the complex, such as at least the first and second proteins of the complex), isolated nucleic acid, host cell or host organism or progeny thereof as taught herein, or composition(s) or formulation(s) comprising such. The components of the kits may be in various forms, such as, e.g., lyophilised, free in solution or immobilised on a solid phase. They may be, e.g., provided in a multi-well plate or as an array or microarray, or they may be packaged separately and/or individually. The may be suitably labelled as taught herein. The kits may be advantageously employed in various applications, such as inter alia in therapeutic, diagnostic and compound-screening applications.

Further provided is:
any isolated complex, isolated nucleic acid, host cell or host organism or progeny thereof as taught herein, or composition(s) or formulation(s) comprising such, for use as a medicament; or for use in the treatment of a disease or condition associated with a retrovirus;
use of any isolated complex, isolated nucleic acid, host cell or host organism or progeny thereof as taught herein, or composition(s) or formulation(s) comprising such for the manufacture of a medicament for the treatment of a disease or condition associated with a retrovirus; or
a method for treating a disease or condition associated with a retrovirus in a subject in need of such treatment, comprising administering to said subject a therapeutically or prophylactically effective amount of any isolated complex, isolated nucleic acid, host cell or host organism or progeny thereof as taught herein, or composition(s) or formulation(s) comprising such;
   wherein the disease or condition associated with a retrovirus is preferably a disease or condition associated with a human retrovirus including human pathogenic and non-pathogenic retrovirus, more preferably HIV or HTLV.

The invention further relates to a complex-binding agent capable of specifically binding to any complex as taught herein. In particular, a complex-binding agent may specifically bind to any isolated complex as intended herein, and/or to any one endogenous complex comprising, consisting substantially of or consisting of the first and second proteins as taught herein. The agent may bind specifically to the complex substantially to the exclusion of one or more or all individual constituents of the complex, preferably substantially to the exclusion of at least the first and/or second proteins of the complex, more preferably substantially to the exclusion of at least the first and second proteins of the complex. Without limitation, the complex-binding agent may be capable of specifically binding to the complex in vitro, in a cell, in an organ and/or in an organism. In an embodiment, the complex-binding agent may be chosen from the group comprising or consisting of an antibody, aptamer, photoaptamer, protein, polypeptide, peptide, nucleic acid, peptidomimetic and small molecule. Particularly preferred complexes for binding the complex-binding agents are those as described herein comprising TRAF2, LNX2, MIZF or TSC22D4, which display particularly advantageous effects in retroviral infection.

Complex-binding agents as intended herein may find various uses, such as without limitation they may be used for detecting the respective complexes (to this aim the complex-binding agents may be preferably detectably labelled), or they may be used for modulating the activity and/or level of the respective complexes, such as for example for the purposes of treatment.

Also contemplated is a method (a screening assay) for selecting the complex-binding agent capable of specifically binding to any complex as taught herein, comprising: (a) providing one or more, preferably a plurality of, test complex-binding agents; (b) selecting from the test complex-binding agents of (a) those which bind to the complex; and (c) counter-selecting (i.e., removing) from the test complex-binding agents selected in (b) those which bind to any one or more individual constituents of the complex, preferably those which bind to at least the first and/or second proteins of the complex, more preferably those which bind to at least the first and second proteins of the complex.

Binding between test complex-binding agents and the complex or its individual constituents may be advantageously tested by contacting (i.e., combining, exposing or incubating) said complex or its individual constituents with the test complex-binding agents under conditions generally conducive for such binding. For example and without limitation, binding between test complex-binding agents and the complex or its individual constituents may be suitably tested in vitro; or may be tested in host cells or host organisms comprising the complex or one or more of its individual constituents and exposed to or configured to express the test complex-binding agents.

The invention further provides a complex-modulating agent capable of modulating the activity and/or level of any complex as taught herein. In particular, a complex-modulating agent may modulate the activity and/or level of any isolated complex as intended herein, and/or of any one endogenous complex comprising, consisting substantially of or consisting of the first and second proteins as taught herein. Without limitation, the complex-modulating agent may be capable of modulating the activity and/or level of the complex in vitro, in a cell, in an organ and/or in an organism. In an embodiment, the complex-modulating agent may be selected from among the complex-binding agents as taught herein. In an embodiment, the complex-modulating agent may be chosen from the group comprising or consisting of an antibody, aptamer, photoaptamer, protein, polypeptide, peptide, nucleic acid, peptidomimetic and small molecule. Particularly preferred complex-modulating agents may be directed to complexes as described herein comprising TRAF2, LNX2, MIZF or TSC22D4, which display particularly advantageous effects in retroviral infection.

Complex-modulating agents as intended herein may find various uses, such as without limitation they may be used for modulating the activity and/or level of the respective complexes for the purposes of treatment.

Also contemplated is a method (a screening assay) for selecting the complex-modulating agent capable of modulating the activity and/or level of any complex as taught herein, comprising: (a) providing one or more, preferably a plurality of, test complex-modulating agents; and (b) selecting from the test complex-modulating agents of (a) those which modulate the activity and/or level of the complex.

Modulation of the activity and/or level of the complex by test complex-modulating agents may be advantageously tested by contacting (i.e., combining, exposing or incubating) said complex with the test complex-modulating agents under conditions generally conducive for such modulation. By means of example and not limitation, where modulation of the activity and/or level of the complex results from binding of the test complex-modulating agents to the complex, said conditions may be generally conducive for such binding. For example and without limitation, modulation of the activity and/or level of the complex by test complex-modulating agents may be suitably tested in vitro; or may be tested in host cells or host organisms comprising the complex and exposed to or configured to express the test complex-modulating agents.

The herein disclosed complexes play an important role in retroviral biology and/or pathogenicity. Also encompassed are thus methods (screening assays) for selecting, from one or more and preferably a plurality of test agents, a candidate therapeutic agent useful in the treatment of a disease or condition associated with a retrovirus, preferably a human retrovirus including human pathogenic and non-pathogenic retrovirus, more preferably HIV or HTLV, comprising the respective steps to determine whether a test agent is capable of specifically binding to the complex and/or of modulating the activity and/or level of the complex.

Further, inasmuch the complex-binding- or complex-modulating agent as intended herein may be an antibody, the invention also teaches a method for immunising an animal using any isolated complex as taught herein, optionally and preferably further comprising isolating an immune serum from so-immunised animal or isolating from so-immunised animal an antibody-producing cell producing an antibody specifically binding to the complex, and optionally and preferably producing a hybridoma from said antibody-producing cell. Further provided is an immune serum, an antibody-producing cell, a hybridoma or antibody reagent isolated or derived from so-immunised animals.

Given that the present complexes are important to retroviral biology and/or pathogenicity, the Applicant also contemplates therapeutic approaches which rely on modulating the activity and/or level of the herein identified host interactor proteins participating in said complexes.

Hence, the invention also provides a host interactor-modulating agent capable of modulating the activity and/or level of any one or more host interactor proteins as taught herein, preferably wherein the host interactor protein is chosen from proteins defined as the ‘second protein’ in the herein disclosed complexes, also preferably wherein the host interactor protein is chosen from proteins defined as the ‘second protein’ in any one of embodiments (i) to (lxxxii) set forth above. In particular, a host interactor-modulating agent may modulate the activity and/or level of any isolated host interactor protein, and/or of any endogenous host interactor protein. Without limitation, the host interactor-modulating agent may be capable of modulating the activity and/or level of the host interactor protein in vitro, in a cell, in an organ and/or in an organism. In an embodiment, the host interactor-modulating agent may be a host interactor-binding agent capable of specifically binding to a host interactor protein, which is thus also disclosed per se (optionally and preferably, host interactor-binding agents may be detectably labelled, allowing their use to detect their corresponding host interactor proteins). In an embodiment, the host interactor-modulating- and/or host interactor-binding agent may be chosen from the group comprising or consisting of an antibody, aptamer, photoaptamer, protein, polypeptide, peptide, nucleic acid, peptidomimetic and small molecule.

Particularly preferred host interactor-modulating agents may be those capable of inhibiting the interactions between TRAF2, LNX2, MIZF or TSC22D4 and HIV and/or HTLV proteins, and/or modulating the activity and/or level of TRAF2, LNX2, MIZF or TSC22D4, which display particularly advantageous effects in retroviral infection. By means of example and not limitation, and as follows from the example sections, beneficial effects in retroviral infections, preferably in HIV and/or HTLV infections, can be obtained through maintaining or increasing the activity and/or level of TRAF2, reducing the activity and/or level of LNX2, reducing the activity and/or level of MIZF (which may counteract viral expression), or increasing the activity and/or level of MIZF (which may stimulate the reactivation of latent virus, thereby making the latter accessible to anti-viral treatments).

Further intended is a method (a screening assay) for selecting the host interactor-modulating agent capable of modulating the activity and/or level of any one or more host interactor proteins as taught herein, comprising: (a) providing one or more, preferably a plurality of, test host interactor-modulating agents; and (b) selecting from the test host interactor-modulating agents of (a) those which modulate the activity and/or level of the one or more host interactor proteins. Modulation of the activity and/or level of the host interactor protein(s) by test host interactor-modulating agents may be advantageously tested by contacting (i.e., combining, exposing or incubating) said host interactor protein(s) with the test host interactor-modulating agents under conditions generally conducive for such modulation. By means of example and not limitation, where modulation of the activity and/or level of the host interactor protein(s) results from binding of the test host interactor-modulating agents to the host interactor protein(s), said conditions may be generally conducive for such binding. For example and without limitation, modulation of the activity and/or level of the host interactor protein(s) by test host interactor-modulating agents may be suitably tested in vitro; or may be tested in host cells or host organisms comprising the host interactor protein(s) and exposed to or configured to express the test host interactor-modulating agents.

As well contemplated is a method (a screening assay) for selecting the host interactor-binding agent capable of specifically binding to any one or more host interactor proteins as taught herein, comprising: (a) providing one or more, preferably a plurality of, test host interactor-binding agents; and (b) selecting from the test host interactor-binding agents of (a) those which specifically bind to the one or more host interactor proteins. Binding between test host interactor-binding agents and the host interactor protein(s) may be advantageously tested by contacting (i.e., combining, exposing or incubating) said host interactor protein(s) with the test host interactor-binding agents under conditions generally conducive for such binding. For example and without limitation, binding between test host interactor-binding agents and the host interactor protein(s) may be suitably tested in vitro; or may be tested in host cells or host organisms comprising the host interactor protein(s) and exposed to or configured to express the test host interactor-binding agents.

Also encompassed are methods (screening assays) for selecting, from one or more and preferably a plurality of test agents, a candidate therapeutic agent useful in the treatment of a disease or condition associated with a retrovirus, preferably a human retrovirus including human pathogenic and non-pathogenic retrovirus, more preferably HIV or HTLV, comprising the respective steps to determine whether a test agent is capable of specifically binding to one or more host interactor proteins and/or capable of modulating the activity and/or level of one or more host interactor proteins.

Further disclosed are compositions and formulations comprising any one or more agents as taught herein, such as any one or more complex-binding agents, complex-modulating agents, host interactor-modulating agents, host interactor-binding agents and/or therapeutic agents selected there from as taught herein, and one or more additional components, such as without limitation one or more solvents and/or one or more pharmaceutically acceptable carriers. Further provided are methods for producing the above compositions or formulations, comprising admixing said one or more agents with one or more additional components.

Particularly intended are pharmaceutical compositions and formulations comprising any one or more agents as taught herein and one or more pharmaceutically acceptable carriers; and methods for producing said pharmaceutical compositions and formulations, comprising admixing said one or more agents with said one or more pharmaceutically acceptable carriers.

As well disclosed are kits of parts comprising any one or more agents as taught herein, such as any one or more complex-binding agents, complex-modulating agents, host interactor-modulating agents, host interactor-binding agents and/or therapeutic agents selected there from as taught herein, or composition(s) or formulation(s) comprising such. The components of the kits may be in various forms, such as, e.g., lyophilised, free in solution or immobilised on a solid phase. They may be, e.g., provided in a multi-well plate or as an array or microarray, or they may be packaged separately and/or individually. The kits may be advantageously employed in various applications, such as inter alia in therapeutic, diagnostic and compound-screening applications.

Further provided is:
any one or more agents as taught herein, such as any one or more complex-binding agents, complex-modulating agents, host interactor-modulating agents, host interactor-binding agents and/or therapeutic agents selected there from as taught herein, or composition(s) or formulation(s) comprising such, for use as a medicament; or for use in the treatment of a disease or condition associated with a retrovirus;
use of any one or more agents as taught herein, such as any one or more complex-binding agents, complex-modulating agents, host interactor-modulating agents, host interactor-binding agents and/or therapeutic agents selected there from as taught herein, or composition(s) or formulation(s) comprising such for the manufacture of a medicament for the treatment of a disease or condition associated with a retrovirus; or
a method for treating a disease or condition associated with a retrovirus in a subject in need of such treatment, comprising administering to said subject a therapeutically or prophylactically effective amount of any one or more agents as taught herein, such as any one or more complex-binding agents, complex-modulating agents, host interactor-modulating agents, host interactor-binding agents and/or therapeutic agents selected there from as taught herein, or composition(s) or formulation(s) comprising such;
   wherein the disease or condition associated with a retrovirus is preferably a disease or condition associated with a human retrovirus including human pathogenic and non-pathogenic retrovirus, more preferably HIV or HTLV.

Given that the herein disclosed complexes and host interactor proteins play an important role in retroviral biology and/or pathogenicity, the Applicant also realises advantageous diagnostic, prognostic and/or predictive approaches relying on said complexes and/or host interactor proteins.

Thus, the invention further relates to a method for diagnosing, predicting and/or prognosticating a disease or condition associated with a retrovirus in a subject, characterised in that the examination phase of the method comprises determining or measuring the structure, activity and/or level of:
any complex as taught herein, in particular any endogenous complex comprising, consisting substantially of or consisting of the first and second proteins as taught herein; and/or
any host interactor protein as taught herein, in particular any endogenous host interactor protein, preferably wherein the host interactor protein is chosen from proteins defined as the ‘second protein’ in the herein disclosed complexes, also preferably wherein the host interactor protein is chosen from proteins defined as the ‘second protein’ in any one of embodiments (i) to (lxxxii) set forth above;
   wherein the disease or condition associated with a retrovirus is preferably a disease or condition associated with a human retrovirus including human pathogenic and non-pathogenic retrovirus, more preferably HIV or HTLV.

Said method for diagnosing, predicting and/or prognosticating the disease or condition associated with a retrovirus in a subject may thus comprise the steps:

(a) determining or measuring the structure, activity and/or level of any complex and/or any host interactor protein as taught herein in a sample from the subject;
(b) comparing the structure, activity and/or level of said complex and/or said host interactor protein determined or measured in (a) with a reference value of the structure, activity and/or level of said complex and/or said host interactor protein, said reference value representing a known diagnosis, prediction and/or prognosis of the disease or condition associated with a retrovirus;
(c) finding a deviation or no deviation of the structure, activity and/or level of said complex and/or said host interactor protein determined or measured in (a) from the reference value;
(d) attributing said finding of deviation or no deviation to a particular diagnosis, prediction and/or prognosis of the disease or condition associated with a retrovirus in the subject.

In accordance with Tables 7 and 8 set forth above and Table 13 below, also disclosed are methods for modulating HIV Tat-mediated transactivation of HIV viral promoter sequences or HTLV Tax-mediated transactivation of HTLV viral promoter sequences, by modulating the activity and/or level of one or more host interactor proteins chosen from proteins (where applicable, defined as the ‘second protein’) in said Tables, such as particularly embodiments (xxxvi) to (liii) set forth in Tables 7, 8. In particular, said modulation of transactivation may be performed in vitro, in a cell, in an organ and/or in an organism. Without limitation, said modulation may be achieved using respective host interactor-modulating agents or host interactor-binding agents.

As noted, to identify the present host interactors of retroviral proteins, the Applicant devised a systematic unbiased binary interactome mapping strategy. Advantageously, this strategy recognises the fact that numerous structurally and/or functionally equivalent proteins may exist between distinct viral species, types or strains, and even within a same virus. Whereas conventional interactome mapping methods frequently overlook interactions involving such structurally and/or functionally equivalent proteins (e.g., because highly overlapping or similar viral ORFs are misidentified by sequence alignment algorithms, or because of the employed pooling techniques), the present strategy minimises this drawback.

Consequently, the invention also comprises a method for identifying interactors of a plurality of (e.g., two or more) query proteins, wherein said query proteins comprise a subgroup of two or more proteins which are structurally and/or functionally similar or equivalent, the method comprising steps:

(a) screening of a plurality of target proteins to identify interactors of the query proteins; and
(b) where a given target protein is identified in step (a) as an interactor of a query protein from said subgroup of query proteins, testing the presence or absence of an interaction between said target protein and one or more or preferably all remaining query proteins from said subgroup of query proteins.

In this manner, reliable and exhaustive information may be obtained about the presence or absence of an interaction between a given target protein (which is found to interact with at least one query protein from the subgroup of structurally and/or functionally similar or equivalent query proteins) and (all) other query proteins from said subgroup. This allows to construct interactomes of the query protein in a systematic and unbiased manner.

The screening of step (a) and testing of step (b) may be performed using any conventional interaction-querying technique, such as without limitation yeast two-hybrid based methods or mass spectrometry (MS) based methods.

Preferably, the screening of step (a) may screen for interactions of the query proteins using two or more pools of query proteins. The use of pools of query proteins accelerates the screening of step (a) but may cause missing more interactions, which is countered by step (b).

Also described are interactome maps and complexes identified using said method.

These and further aspects and preferred embodiments of the invention are described in the following sections and in the appended claims. The subject matter of appended claims 1 to 29 is hereby specifically incorporated in this specification.

### BRIEF DESCRIPTION OF FIGURES

FIG. 1 illustrates the pipeline of the HT-Y2H experiment. Viral ORFeome have been screened against Human ORFeome 3.1 in both configurations (DB-hORF AD-vORF and DB-vORF AD-hORF). Interactions from the primary screen have been used to perform an homologous individual retest, i.e., any human interactor of a viral protein was also retested for interaction with all homologous viral proteins, as well as proteins susceptible to play a similar role. For example, if HIV-1 Tat protein has been found to interact with human HOXD3 protein in the primary screen, all Tat, Tax, Rev and Rex proteins will be retested for interaction with HOXD3. To guarantee a high level of specificity, only interactions identified with at least two out of three phenotypes were considered positive.

FIG. 2 illustrates HIV-HTLV virus-host Y2H PPI network. Viral ORFs are pictured as big diamonds; human ORFs are shown as small circles. The links between viral and human proteins represent interactions discovered in our Y2H screen.

FIG. 3 illustrates targeting of Notch signalling pathway by viral proteins. Viral ORFs are pictured as big diamonds; human ORFs are shown as small circles.

FIG. 4 illustrates targeting of apoptosis signalling pathway by viral proteins. Viral ORFs are pictured as big diamonds; human ORFs are shown as small circles.

FIG. 5 illustrates that interaction with HIV1-Gag results in disruption of the TRAF2 complex.

FIG. 6 illustrates the effect of HIV1-Gag on degradation of TRAF2.

FIG. 7 illustrates that LNX2 knock-down affects HIV-1 expression and Tat transactivation in TZM-bl cell line. (A) Verification of LNX2 knock-down was done by western blot using an anti-LNX2 antibody (Santa Cruz Biotechnology). Actin was used as a loading control. (B) TZM-bl-control and TZM-bl-KO-shLNX2 were infected with the X4-tropic HIV-1NL4.3 viral strain (MOI, 5) for 24 hours and beta-galactosidase activities were measured. (C) TZM-bl-control and TZM-bl-KO-shLNX2 were transfected with increasing amounts of HIV1 Tat expressing construct and luciferase activities measured.

FIG. 8 illustrates (A) Western-blotting of HEK293T cell extracts transfected with expressing vectors for GST-TRAF2 or truncated mutants deltaTRAF2 and deltaRING and p55 GAG/GFP, as indicated. Cell extracts were immunobloted with anti-GST, anti-GFP and anti-actin antibodies. (B) Western-blotting of HEK293T cells transfected with expressing vectors for GST-TRAF2 or GST-deltaRING truncated mutant, p55 GAG/GFP and Myc-Ubiquitin, as indicated. Cell extracts were immunobloted with anti-GST, anti-Myc and anti-actin antibodies. (C) Cells were pretreated with the cathepsin B inhibitor CA-074Me (10 μM) or the proteasomal inhibitor MG-132 (1 μM) for 24 H. Cell lysates were analysed as in (B).

FIG. 9 illustrates relative HTLV1-HBZ, -Gag and -Tax mRNA expression following MT4 cells treatment with vehicle (0.5% DMSO) or 1 μM of γ-secretase inhibitor L685,458. Viral mRNA expression data are calculated relative to GAPDH mRNA expression data as 2̂(CT(GAPDH)-CT(HBZ/Gag/Tax)) over three times triplicate experiments for each gene.

FIG. 10 illustrates HIV p24 expression in latently infected Jurkat T cell lines (Jlat8.4; 9.2 and 15.4) lacking MIZF.

FIG. 11 illustrates HIV promoter activation following knock down of TRAF2 in TZM-bl HIV-indicator cell lines. TZM-bl cells (expressing a luciferase gene under the control of HIV-1 promoter) were treated by shRNA targeting TRAF2 mRNA. Data are relative luciferase counts from three independent experiments in triplicate.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

All documents cited in the present specification are hereby incorporated by reference in their entirety.

Unless otherwise defined, all terms used in disclosing the invention, including technical and scientific terms, have the meaning as commonly understood by one of ordinary skill in the art to which this invention belongs. By means of further guidance, term definitions may be included to better appreciate the teaching of the present invention.

For general methods relating to the invention, reference is made inter alia to well-known textbooks, including, e.g., “Molecular Cloning: A Laboratory Manual, 2nd Ed.” (Sambrook et al., 1989), Animal Cell Culture (R. I. Freshney, ed., 1987), the series Methods in Enzymology (Academic Press), Gene Transfer Vectors for Mammalian Cells (J. M. Miller & M. P. Calos, eds., 1987); “Current Protocols in Molecular Biology and Short Protocols in Molecular Biology, 3rd Ed.” (F. M. Ausubel et al., eds., 1987 & 1995); Recombinant DNA Methodology II (R. Wu ed., Academic Press 1995).

General techniques in cell culture and media uses are outlined inter alia in Large Scale Mammalian Cell Culture (Hu et al. 1997. Curr Opin Biotechnol 8: 148); Serum-free Media (K. Kitano. 1991. Biotechnology 17: 73); or Large Scale Mammalian Cell Culture (Curr Opin Biotechnol 2: 375, 1991).

The term “complex” may generally denote an association (e.g., a comparably transient or permanent association) of two or more interacting constituents. A constituent may thus be involved in a complex through its interacting with one or more other constituents of said complex. Preferably, interactions between the constituents of a complex may be non-covalent, including primarily but without limitation van der Waals interactions, electrostatic (ionic) interactions, hydrogen bonds and/or hydrophobic packing. Preferably, a complex as intended herein may be a macromolecular complex.

In the present context, constituents of a complex may primarily encompass molecules, more preferably biomolecules, even more preferably proteins. The term “protein” as used herein generally refers to macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native protein, such as, e.g., amino acid deletions, additions and/or substitutions. The term contemplates both full-length proteins and protein parts or fragments, e.g., naturally-occurring protein parts that ensue from processing of such full-length proteins.

The term “isolated” with reference to a particular component (such as for instance a protein or a complex) generally denotes that such component exists in separation from—for example, has been separated from or prepared and/or maintained in separation from—one or more other components of its natural environment. For instance, an isolated human or animal protein or complex may exist in separation from a human or animal body where it naturally occurs.

The term “isolated” as used herein may preferably also encompass the qualifier “purified”. By means of example, the term “purified” with reference to proteins or complexes does not require absolute purity. Instead, it denotes that such proteins or complexes are in a discrete environment in which their abundance (conveniently expressed in terms of mass or weight or concentration) relative to other proteins or complexes is greater than in a biological sample. A discrete environment denotes a single medium, such as for example a single solution, gel, precipitate, lyophilisate, etc. Purified proteins or complexes may be obtained by known methods including, for example, laboratory or recombinant synthesis, chromatography, preparative electrophoresis, centrifugation, precipitation, affinity purification, etc.

Purified proteins or complexes may preferably constitute by weight ≧about 10%, more preferably ≧about 50%, such as ≧about 60%, yet more preferably ≧about 70%, such as ≧about 80%, and still more preferably ≧about 90%, such as ≧about 95%, ≧about 96%, ≧about 97%, ≧about 98%, ≧about 99% or even 100%, of the protein content of the discrete environment. Protein content may be determined, e.g., by the Lowry method (Lowry et al. 1951. J Biol Chem 193: 265), optionally as described by Hartree 1972 (Anal Biochem 48: 422-427). Also, purity of proteins or complexes may be determined by SDS-PAGE under reducing or non-reducing conditions using Coomassie blue or, preferably, silver stain.

The term “retrovirus” is used herein in its conventional meaning and generally encompasses a class of viruses in which the genetic material is single-stranded RNA and which employ reverse transcriptase to transcribe the viral RNA into DNA in a host. Retroviruses as intended herein may particularly belong to the viral family Retroviridae, more particularly to sub-families Oncovirinae, Lentivirinae or Spumavirinae. Retroviruses as intended herein may be pathogenic (i.e., causing a demonstrable disease phenotype in an infected host) or may be non-pathogenic (i.e., wherein an infected host's condition does not manifest a demonstrable disease phenotype). Particularly intended herein are retroviruses infecting animals, more preferably retroviruses of warm-blooded animals, even more preferably of vertebrate animals, still more preferably of mammals, yet more preferably of primates, and most preferably of humans. Particularly preferred herein are human retroviruses including without limitation HIV-1, HIV-2, HTLV-1 and HTLV-2.

Reference to “diseases or conditions associated with a retrovirus” generally encompasses any and all states of a host resultant from the host having been infected with the retrovirus. Without limitation, such states may be typified by the presence of viral biological material in the infected host, e.g., the presence of provirus in the genome of one or more cells of the infected host and/or the presence of viral nucleic acids, viral proteins or viral particles in the infected host. Without limitation, such states may comprise stages when the provirus is dormant or latent, pre-clinical stages when virus is produced in the infected host but without demonstrable disease symptoms, as well as clinical stages involving demonstrable disease symptoms, such as for example acquired immunodeficiency syndrome (AIDS) caused by HIV-1 and HIV-2, or adult T-cell leukaemia/lymphoma (ATLL) or tropical spastic paraparesis (TSP) caused by HTLV-1.

Further, the term “protein of a retrovirus” generally encompasses proteins encoded by any open reading frame (ORF) of a retroviral genome. Where a single ORF encodes a pre-protein which is processed into one, two or more mature proteins, the term may encompass both the pre-protein and the processed mature proteins. For example, HIV proteins may be particularly encoded by HIV ORFs: Gag, Env, Pol, Tat, Rev, Nef, Vif, Vpr, Vpu or Vpx known per se. For example, HTLV proteins may be particularly encoded by HTLV ORFs: Gag, Env, Pol, Tax, Rex, HBZ, p30, p13 or p12 known per se.

Sequence data including gene, transcript and protein sequence data for HIV and HTLV ORFs are generally known and can be retrieved from public databases such as for example NCBI GenBank (http://www.ncbi.nlm.nih.gov/). By means of example and not limitation, illustrative sequences of HIV and HTLV ORFs are listed in Table 11 below with associated database accession numbers and information. The sequences deemed as incorporated herein are preferably those found in the respective database entries that are live on the filing date of the present application.

TABLE 11
HIV and HTLV ORFs, accession numbers and database information.

| | | NCBI | | | | |
|---|---|---|---|---|---|---|
| | NCBI | protein | NCBI | Protein | Entrez | |
| ORF | gene name | name | locus tag | accession # | Gene # | Group |
| HIV1_gagpol | gag-pol | Gag-Pol | HIV1gp1 | NP_057849 | 155348 | gag_pol |
| HIV1_gag | gag | Pr55(Gag) | HIV1gp2 | NP_057850 | 155030 | gag_pol |
| HIV1_vif | vif | Vif | HIV1gp3 | NP_057851 | 155459 | |
| HIV1_vpr | vpr | Vpr | HIV1gp4 | NP_057852 | 155807 | |
| HIV1_tat | tat | Tat | HIV1gp5 | NP_057853 | 155871 | tax_tat_rex_rev |
| HIV1_rev | rev | Rev | HIV1gp6 | NP_057854 | 155908 | tax_tat_rex_rev |
| HIV1_vpu | vpu | Vpu | HIV1gp7 | NP_057855 | 155945 | |
| HIV1_env | env | Envelope | HIV1gp8 | NP_057856 | 155971 | env |
| | | surface | | | | |
| | | glycoprotein | | | | |
| | | gp160, | | | | |
| | | precursor | | | | |
| HIV1_nef | nef | Nef | HIV1gp9 | NP_057857 | 156110 | |
| HIV2_gagpol | gag-pol | gag-pol- | HIV2gp1 | NP_663784 | 1490001 | gag_pol |
| | | fusion- | | | | |
| | | polyprotein | | | | |
| HIV2_gag | gag-pol | gag- | HIV2gp1 | NP_056837 | 1490001 | gag_pol |
| | | polyprotein | | | | |
| HIV2_vpr | vpr | vpr protein | HIV2gp4 | NP_056841 | 1724718 | |
| HIV2_tat | tat | tat protein | HIV2gp5 | NP_056842 | 1724713 | tax_tat_rex_rev |
| HIV2_rev | rev | rev protein | HIV2gp6 | NP_056843 | 1724716 | tax_tat_rex_rev |
| HIV2_env | env | env | HIV2gp7 | NP_056844 | 1724717 | env |
| | | polyprotein | | | | |
| HIV2_nef | nef | nef protein | HIV2gp8 | NP_056845 | 1724715 | |
| HTLV1_gag | gag | Pr55 | HTLV1gp3 | NP_057862 | 1491934 | gag_pol |
| HTLV1_pol | pol | Pr pol | HTLV1gp5 | | 1491936 | gag_pol |
| HTLV1_rex | rex | p27 | HTLV1gp6 | NP_057863 | 1491937 | tax_tat_rex_rev |
| HTLV1_tax | tax | p40 | HTLV1gp7 | NP_057864 | 1491938 | tax_tat_rex_rev |
| HTLV1_env | env | gp46 SU | HTLV1gp8 | NP_057865 | 1491939 | env |
| HTLV1_p12 | p12 | P12 | | DQ065793 | | p30 |
| HTLV1_p30 | p30 | P30 | | L08433 | | p30 |
| HTLV1_p13 | p13 | P13 | | L08433 | | p30 |
| HTLV1_hbz | hbz | bZIP factor | | ABB89742 | | p30 |
| HTLV2_gag | HTLV2gp2 | gag | HTLV2gp2 | NP_041002 | 1491944 | gag_pol |
| | | polyprotein | | | | |
| HTLV2_pol | HTLV2gp3 | pol | HTLV2gp3 | NP_041003 | 1491943 | gag_pol |
| | | polyprotein | | | | |
| HTLV2_rex | HTLV2gp4 | rex 26 kD | HTLV2gp4 | NP_041004 | 1491945 | tax_tat_rex_rev |
| HTLV2_tax2 | HTLV2gp5 | tax protein | HTLV2gp5 | NP_041005 | 1491946 | tax_tat_rex_rev |
| HTLV2_env | HTLV2gp6 | env | HTLV2gp6 | NP_041006 | 1491942 | env |
| | | propeptide | | | | |

Host interactor proteins, e.g., proteins referred to as ‘second proteins’ throughout this specification, may encompass such proteins and polypeptides of any organism where found, and particularly of animals, preferably warm-blooded animals, more preferably of vertebrate animals, yet more preferably of mammals, still more preferably of primates, and most preferably of humans. Preferably, the host interactor proteins may be of a host susceptible to an infection by a retrovirus of interest.

The terms particularly encompass such host interactor proteins with a native sequence, i.e., ones of which the primary sequence is the same as that of the proteins found in or derived from nature. A skilled person understands that native sequences of host interactor proteins may differ between different species due to genetic divergence between such species, and/or may differ between or within different individuals of the same species due to normal genetic diversity (variation) within a given species. Also, the native sequences of host interactor proteins may differ between or even within different individuals of the same species due to post-transcriptional or post-translational modifications. Accordingly, all host interactor protein sequences found in or derived from nature are considered “native”. The terms encompass the host interactor proteins when forming a part of a living organism, organ, tissue or cell, when forming a part of a biological sample, as well as when at least partly isolated from such sources. The terms also encompass proteins when produced by recombinant or synthetic means.

Sequence data including gene, transcript and protein sequence data for host interactor proteins intended herein, e.g., proteins referred to as ‘second proteins’ throughout this specification, are generally known and can be retrieved from public databases such as for example NCBI GenBank. By means of example and not limitation, illustrative sequences of human host interactor proteins are listed in Table 12 below, listing Gene ID numbers uniquely identifying said host interactors in “Entrez Gene” database of NCBI (described in Maglott et al. 2005. Entrez Gene: gene-centered information at NCBI. Nucleic Acids Res. 33: D54-D58). Where the below Gene ID numbers directly or indirectly (e.g., by referring to another database) encompass sequence information, the sequences deemed as incorporated herein are preferably those found in the respective database entries that are live on the filing date of the present application.

TABLE 12
Human host interactor proteins and Gene ID accession numbers.

| Host interactor | Gene ID |
|---|---|
| AP4M1 | 9179 |
| ARL6IP4 | 51329 |
| BACH1 | 571 |
| BHLHB2 | 8553 |
| C10orf3 | 55165 |
| C16orf33 | 79622 |
| C1orf59 | 113802 |
| C1orf94 | 84970 |
| C20orf141 | 128653 |
| C20orf81 | 64773 |
| C8orf32 | 55093 |
| C8orf56 | 157556 |
| CCDC24 | 149473 |
| CDC23 | 8697 |
| Cep63 | 80254 |
| Cep70 | 80321 |
| CHCHD3 | 54927 |
| CRADD | 8738 |
| CRSP9 | 9443 |
| CRX | 1406 |
| CTDSP2 | 10106 |
| CTNNBIP1 | 56998 |
| DAZAP2 | 9802 |
| DDX6 | 1656 |
| DGCR6L | 85359 |
| DHRS10 | 51171 |
| DIPA | 11007 |
| DLAT | 1737 |
| Dlc2 | 140735 |
| DLX2 | 1746 |
| DVL2 | 1856 |
| ETV4 | 2118 |
| EWSR1 | 2130 |
| FANCG | 2189 |
| FATE1 | 89885 |
| FKBP7 | 51661 |
| FLJ10726 | 55216 |
| FLJ20097 | 55610 |
| FLJ22471 | 80212 |
| FLJ25439 | 153657 |
| FLJ32855 | 80125 |
| FTH1 | 2495 |
| FXR2 | 9513 |
| GADD45GIP1 | 90480 |
| GCC1 | 79571 |
| GOPC | 57120 |
| HGS | 9146 |
| HOXA3 | 3200 |
| HOXB9 | 3219 |
| HOXD3 | 3232 |
| HSFY1 | 86614 |
| KCTD1 | 284252 |
| KIAA0258 | 9827 |
| KIAA1683 | 80726 |
| KIAA1949 | 170954 |
| KIF9 | 64147 |
| KLC3 | 147700 |
| KLHL12 | 59349 |
| KRT15 | 3866 |
| KRT4 | 3851 |
| KRT6A | 3853 |
| KRT8 | 3856 |
| L3MBTL3 | 84456 |
| LDOC1 | 23641 |
| LENG1 | 79165 |
| LNX2 | 222484 |
| LOC283385 | 283385 |
| LOC388818 | 388818 |
| LOC391257 | 391257 |
| LOC541468 | 541468 |
| LOC595101 | 595101 |
| LOC91661 | 91661 |
| LZTS2 | 84445 |
| MAD1L1 | 8379 |
| MAD2L2 | 10459 |
| MEIS2 | 4212 |
| MGC11257 | 84310 |
| MGC27019 | 150483 |
| MGC4266 | 84766 |
| MINA | 84864 |
| MIZF | 25988 |
| MKRN3 | 7681 |
| MLLT11 | 10962 |
| MLX | 6945 |
| MRPS6 | 64968 |
| MYEF2 | 50804 |
| MYST2 | 11143 |
| NEFL | 4747 |
| NIF3L1 | 60491 |
| NKAP | 79576 |
| NOS3 | 4846 |
| NUDT18 | 79873 |
| NUP62 | 23636 |
| OTX2 | 5015 |
| PCBP1 | 5093 |
| PDE9A | 5152 |
| PIAS2 | 9063 |
| PLEKHF2 | 79666 |
| POLM | 27434 |
| POP5 | 51367 |
| PSMA1 | 5682 |
| PSMF1 | 9491 |
| RBPMS | 11030 |
| RFX4 | 5992 |
| RIBC2 | 26150 |
| RNPS1 | 10921 |
| SF3A3 | 10946 |
| SFRS11 | 9295 |
| SLC2A4 | 6517 |
| SORBS3 | 10174 |
| SOX5 | 6660 |
| SP100 | 6672 |
| SPAG5 | 10615 |
| SPG21 | 51324 |
| SS18L1 | 26039 |
| SSX2IP | 117178 |
| TEX11 | 56159 |
| TFIP11 | 24144 |
| TRAF2 | 7186 |
| TRIP6 | 7205 |
| TSC22D4 | 81628 |
| TTC23 | 64927 |
| UBE2I | 7329 |
| VPS28 | 51160 |
| WDFY3 | 23001 |
| ZBTB16 | 7704 |
| ZCCHC7 | 84186 |
| ZMAT1 | 84460 |
| ZNF581 | 51545 |
| ZNF614 | 80110 |
| ZNHIT4 | 83444 |

Where a reference is made herein to a protein or polypeptide, such reference is to be understood as also encompassing fragments and/or variants of said protein or polypeptide, particularly including functional fragments and/or variants of said protein or polypeptide.

The term “fragment” generally denotes a N- and/or C-terminally truncated form of a protein or polypeptide. Preferably, a fragment may comprise at least about 30%, e.g., at least 50% or at least 70%, preferably at least 80%, e.g., at least 85%, more preferably at least 90%, and yet more preferably at least 95% or even about 99% of the amino acid sequence length of said protein or polypeptide.

The term “variant” of a given recited protein or polypeptide refers to proteins or polypeptides the amino acid sequence of which is substantially identical (i.e., largely but not wholly identical) to the sequence of said recited protein or polypeptide, e.g., at least about 85% identical, e.g., preferably at least about 90% identical, e.g., at least 91% identical, 92% identical, more preferably at least about 93% identical, e.g., 94% identical, even more preferably at least about 95% identical, e.g., at least 96% identical, yet more preferably at least about 97% identical, e.g., at least 98% identical, and most preferably at least 99% identical. Preferably, a variant may display such degrees of identity to a recited protein or polypeptide when the whole sequence of the recited protein is queried in the sequence alignment (i.e., overall sequence identity).

Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Left 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62, cost to open a gap=11, cost to extend a gap=1, expectation value=10.0, word size=3).

In an embodiment, a variant of a given protein or polypeptide may be a homologue (e.g., orthologue or paralogue) of said protein or polypeptide. As used herein, the term “homology” generally denotes structural similarity between two macromolecules, particularly between two proteins or polypeptides or polynucleotides, from same or different taxons, wherein said similarity is due to shared ancestry.

The term “functional” denotes that fragments and/or variants at least partly retain the biological activity or functionality of the recited proteins or polypeptides. Preferably, such functional fragments and/or variants may retain at least about 20%, e.g., at least 30%, or at least 40%, or at least 50%, e.g., at least 60%, more preferably at least 70%, e.g., at least 80%, yet more preferably at least 85%, still more preferably at least 90%, and most preferably at least 95% or even 100% or higher of the activity compared to the corresponding recited proteins or polypeptides. For example, such functional fragments and/or variants may retain one or more aspects of the biological activity of the recited proteins or polypeptides, such as, e.g., ability to participate in a complex, ability to participate in a cellular pathway, etc.

The term “nucleic acid” as used herein means a polymer of any length composed essentially of nucleotides, e.g., deoxyribonucleotides and/or ribonucleotides. Nucleic acids can comprise purine and/or pyrimidine bases and/or other natural (e.g., xanthine, inosine, hypoxanthine), chemically or biochemically modified (e.g., methylated), non-natural, or derivatised nucleotide bases. The backbone of nucleic acids can comprise sugars and phosphate groups, as can typically be found in RNA or DNA, and/or one or more modified or substituted sugars (such as, e.g., 2′-O-alkylated, e.g., 2′-O-methylated or 2′-O-ethylated; or 2′-O,4′-C-alkynelated, e.g., 2′-O,4′-C-ethylated sugars) and/or one or more modified or substituted phosphate groups (e.g., phosphodiester, phosphorothioate, phosphorodithioate, methylphosphonate, phosphoramidate, alkyl phosphotriester, sulfamate, 3′-thioacetal, methylene (methylimino), 3′-N-carbamate, morpholino carbamate, and peptide nucleic acids (PNAs)). The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g. chemically synthesised) DNA, RNA or DNA/RNA hybrids. A nucleic acid can be naturally occurring, e.g., present in or isolated from nature, can be recombinant, i.e., produced by recombinant DNA technology, and/or can be, partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

By “encoding” is meant that a nucleic acid sequence or part(s) thereof corresponds, by virtue of the genetic code of an organism in question to a particular amino acid sequence, e.g., the amino acid sequence of one or more desired proteins or polypeptides.

Preferably, a nucleic acid encoding one or more proteins or polypeptides (e.g., one or more proteins participating in complexes as taught herein) may comprise an open reading frame (ORF) encoding said protein or polypeptide. An “open reading frame” or “ORF” refers to a succession of coding nucleotide triplets (codons) starting with a translation initiation codon and closing with a translation termination codon known per se, and not containing any internal in-frame translation termination codon, and potentially capable of encoding a protein or polypeptide. Hence, the term may be synonymous with “coding sequence” as used in the art.

Expression of proteins can be achieved through operably linking nucleic acid sequences or ORFs encoding said proteins with regulatory sequences allowing for expression of the nucleic acids or ORFs, e.g., in vitro, in a host cell, host organ and/or host organism. Such expression may be achieved, e.g., under suitable (culture) conditions or upon addition of inducers (e.g., where inducible regulatory sequences are used).

An “operable linkage” is a linkage in which regulatory sequences and sequences sought to be expressed are connected in such a way as to permit said expression. For example, sequences, such as, e.g., a promoter and an ORF, may be said to be operably linked if the nature of the linkage between said sequences does not: (1) result in the introduction of a frame-shift mutation, (2) interfere with the ability of the promoter to direct the transcription of the ORF, (3) interfere with the ability of the ORF to be transcribed from the promoter sequence.

The precise nature of regulatory sequences or elements required for expression may vary between expression environments, but typically include a promoter and a transcription terminator, and optionally an enhancer.

Reference to a “promoter” or “enhancer” is to be taken in its broadest context and includes transcriptional regulatory sequences required for accurate transcription initiation and where applicable accurate spatial and/or temporal control of gene expression or its response to, e.g., internal or external (e.g., exogenous) stimuli. More particularly, “promoter” may depict a region on a nucleic acid molecule, preferably DNA molecule, to which an RNA polymerase binds and initiates transcription. A promoter is preferably, but not necessarily, positioned upstream, i.e., 5′, of the sequence the transcription of which it controls. Typically, in prokaryotes a promoter region may contain both the promoter per se and sequences which, when transcribed into RNA, will signal the initiation of protein synthesis (e.g., Shine-Dalgarno sequence).

In embodiments, promoters contemplated herein may be constitutive or inducible.

The terms “terminator” or “transcription terminator” refer generally to a sequence element at the end of a transcriptional unit which signals termination of transcription. For example, a terminator is usually positioned downstream of, i.e., 3′ of ORF(s) encoding a polypeptide of interest. For instance, where a recombinant nucleic acid contains two or more ORFs, e.g., successively ordered and forming together a multi-cistronic transcription unit, a transcription terminator may be advantageously positioned 3′ to the most downstream ORF.

The term “vector” generally refers to a nucleic acid molecule, typically DNA, to which nucleic acid segments may be inserted and cloned, i.e., propagated. Hence, a vector will typically contain one or more unique restriction sites, and may be capable of autonomous replication in a defined host or vehicle organism such that the cloned sequence is reproducible. Vectors may include, without limitation, plasmids, phagemids, bacteriophages, bacteriophage-derived vectors, PAC, BAC, linear nucleic acids, e.g., linear DNA, viral vectors, etc., as appropriate. Expression vectors are generally configured to allow for and/or effect the expression of nucleic acids or ORFs introduced thereto in a desired expression system, e.g., in vitro, in a host cell, host organ and/or host organism. For example, expression vectors may advantageously comprise suitable regulatory sequences.

The terms “host cell” and “host organism” may suitably refer to cells or organisms encompassing both prokaryotes, such as bacteria, and eukaryotes, such as yeast, fungi, protozoan, plants and animals. Contemplated as host cells are inter alia unicellular organisms, such as bacteria (e.g., E. coli, Salmonella tymphimurium, Serratia marcescens, or Bacillus subtilis), yeast (e.g., Saccharomyces cerevisiae or Pichia pastoris), (cultured) plant cells (e.g., from Arabidopsis thaliana or Nicotiana tobaccum) and (cultured) animal cells (e.g., vertebrate animal cells, mammalian cells, primate cells, human cells or insect cells). Contemplated as host organisms are inter alia multi-cellular organisms, such as plants and animals, preferably animals, more preferably warm-blooded animals, even more preferably vertebrate animals, still more preferably mammals, yet more preferably primates; particularly contemplated are such animals and animal categories which are non-human.

The terms “sample” or “biological sample” as used herein include any biological specimen obtained from a biological source, such as a subject. Preferred samples may include ones comprising the present complexes or host interactor proteins in detectable quantities. Preferably, the sample may be whole blood or a fractional component thereof such as, e.g., plasma, serum, or a cell pellet. Preferably the sample is readily obtainable by minimally invasive methods. Samples may also include tissue samples and biopsies, tissue homogenates and the like.

As used herein, the term “agent” broadly refers to any chemical (e.g., inorganic or organic), biochemical or biological substance, molecule or macromolecule (e.g., biological macromolecule), a combination or mixture thereof, a sample of undetermined composition, or an extract made from biological materials such as bacteria, plants, fungi, or animal cells or tissues. Preferred though non-limiting “agents” include nucleic acids, oligonucleotides, ribozymes, polypeptides or proteins, a peptides, peptidomimetics, antibodies and fragments and derivatives thereof, aptamers, chemical substances, preferably organic molecules, more preferably small organic molecules, lipids, carbohydrates, polysaccharides, etc., and any combinations thereof.

The term “specifically bind” as used throughout this specification means that an agent binds to one or more desired molecules or analytes, such as to one or more complexes, proteins or polypeptides of interest or fragments or variants thereof substantially to the exclusion of other molecules which are random or unrelated, and optionally substantially to the exclusion of other molecules that are structurally related. Binding of an agent to a target may be evaluated inter alia using conventional interaction-querying methods, such as co-immunoprecipitation, immunoassay methods, chromatography methods, gel elecrophoresis methods, yeast two hybrid methods, or combinations thereof.

The term “specifically bind” does not necessarily require that an agent binds exclusively to its intended target(s). For example, an agent may be said to specifically bind to complex(es), protein(s) or polypeptide(s) of interest or fragments or variants thereof if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, than its affinity for a non-target molecule.

Preferably, the agent may bind to its intended target(s) with affinity constant (K_{A}) of such binding K_{A}≧1×10⁶ M⁻¹, more preferably K_{A}≧1×10⁷ M⁻¹, yet more preferably K_{A}≧1×10⁸ M⁻¹, even more preferably K_{A}≧1×10⁹ M⁻¹, and still more preferably K_{A}≧1×10¹⁰ M⁻¹ or K_{A}≧1×10¹¹ M⁻¹, wherein K_{A}=[A_T]/[A][T], A denotes the agent, T denotes the intended target. Determination of K_{A} can be carried out by methods known in the art, such as for example, using equilibrium dialysis and Scatchard plot analysis. Specific-binding agents as used throughout this specification may include inter alia an antibody, aptamer, photoaptamer, protein, polypeptide, peptide, nucleic acid, peptidomimetic or a small molecule. In an embodiment, a specific-binding agent may be a naturally-occurring binding partner of the target.

As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro, in cell culture, or in vivo.

In an embodiment, an antibody may be any of IgA, IgD, IgE, IgG and IgM classes, and preferably IgG class antibody.

In an embodiment, the antibody may be a polyclonal antibody, e.g., an antiserum or immunoglobulins purified there from (e.g., affinity-purified).

In another preferred embodiment, the antibody may be a monoclonal antibody or a mixture of monoclonal antibodies. Monoclonal antibodies can target a particular antigen or a particular epitope within an antigen with greater selectivity and reproducibility.

By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

In further embodiments, antibody agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments; diabodies; linear antibodies; single-chain antibody molecules; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius) also including camel heavy-chain antibodies V_{H}H, llama (e.g., Lama paccos, Lama glama or Lama vicugna) also including llama heavy-chain antibodies V_{H}H, or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

Methods for immunising animals, e.g., non-human animals such as laboratory or farm animals, using immunising antigens (such as, e.g., the herein disclosed complexes) optionally fused to or covalently or non-covalently linked, bound or adsorbed to a presenting carrier, and preparation of antibody or cell reagents from immune sera is well-known per se and described in documents referred to elsewhere in this specification. The animals to be immunised may include any animal species, preferably warm-blooded species, more preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel, llama or horse. The term “presenting carrier” or “carrier” generally denotes an immunogenic molecule which, when bound to a second molecule, augments immune responses to the latter, usually through the provision of additional T cell epitopes. The presenting carrier may be a (poly)peptidic structure or a non-peptidic structure, such as inter alia glycans, polyethylene glycols, peptide mimetics, synthetic polymers, etc. Exemplary non-limiting carriers include human Hepatitis B virus core protein, multiple C3d domains, tetanus toxin fragment C or yeast Ty particles.

Selection of agents specifically binding to one or more targets of interest to the exclusion of other molecules (non-targets) may suitably involve methods for subtracting or removing from agents that bind to said one or more targets those agents that also cross-react or cross-bind with one or more non-targets. Such subtraction may be readily performed as known in the art by a variety of affinity separation methods, such as affinity chromatography, affinity solid phase extraction, affinity magnetic extraction, etc.

The term “aptamer” refers to single-stranded or double-stranded oligo-DNA, oligo-RNA or oligo-DNA/RNA or any analogue thereof, that can specifically bind to a target molecule. Advantageously, aptamers can display fairly high specificity and affinity (e.g., K_{A} in the order 1×10⁹ M⁻¹) for their targets. Aptamer production is described inter alia in U.S. Pat. No. 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein. The term “photoaptamer” refers to an aptamer that contains one or more photoreactive functional groups that can covalently bind to or crosslink with a target molecule. The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134). The term “small molecule” refers to compounds, preferably organic compounds, with a size comparable to those organic molecules generally used in pharmaceuticals. The term excludes biological macromolecules (e.g., proteins, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

The term “label” or “detectable label” as used throughout this specification refers to any atom, molecule, moiety or biomolecule that can be used to provide a detectable and preferably quantifiable read-out or property, and that can be attached to or made part of an entity of interest, such as a complex, protein, polypeptide or an agent. Labels may be suitably detectable by mass spectrometric, spectroscopic, optical, colorimetric, magnetic, photochemical, biochemical, immunochemical or chemical means. Labels include without limitation dyes; radiolabels such as ³²P, ³³P, ³⁵S, ¹²⁵I, ¹³¹I; electron-dense reagents; enzymes (e.g., horse-radish peroxidase or alkaline phosphatase as commonly used in immunoassays); binding moieties such as biotin-streptavidin; haptens such as digoxigenin; luminogenic, phosphorescent or fluorogenic moieties; mass tags; and fluorescent dyes alone or in combination with moieties that can suppress or shift emission spectra by fluorescence resonance energy transfer (FRET).

The term “modulate” generally denotes a qualitative or quantitative alteration, change or variation specifically encompassing both increase (e.g., activation) or decrease (e.g., inhibition), of that which is being modulated. The term encompasses any extent of such modulation.

For example, where modulation effects a determinable or measurable variable, then modulation may encompass an increase in the value of said variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of said variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation.

Preferably, modulation of the activity and/or level of intended target(s) (e.g., complexes or proteins taught herein) may be specific or selective, i.e., the activity and/or level of intended target(s) may be modulated without substantially altering the activity and/or level of random, unrelated targets.

Reference to the “activity” of a target such as a complex or protein may generally encompass any one or more aspects of the biological activity of the target, such as without limitation any one or more aspects of its biochemical activity, enzymatic activity, signalling activity and/or structural activity, e.g., within a cell, tissue, organ or an organism.

In an embodiment, the activity of a target such as a complex or protein may be modulated and in particular reduced by introducing into or expressing in a cell, tissue, organ or an organism a dominant negative variant of said target, e.g., a dominant negative variant of one or more constituents of the complex, or a dominant negative variant of the protein.

Reference to the “level” of a target such as a complex or protein may preferably encompass the quantity and/or the availability (e.g., availability for performing its biological activity) of the target, e.g., within a cell, tissue, organ or an organism.

For example, the level of a target may be modulated by modulating the target's expression and/or modulating the expressed target. Modulation of the target's expression may be achieved or observed, e.g., at the level of heterogeneous nuclear RNA (hnRNA), precursor mRNA (pre-mRNA), mRNA or cDNA encoding the target. By means of example and not limitation, decreasing the expression of a target may be achieved by methods known in the art, such as, e.g., by transfecting (e.g., by electroporation, lipofection, etc.) or transducing (e.g., using a viral vector) a cell, tissue, organ or organism with an antisense agent, such as, e.g., antisense DNA or RNA oligonucleotide, a construct encoding the antisense agent, or an RNA interference agent, such as siRNA or shRNA, or a ribozyme or vectors encoding such, etc. By means of example and not limitation, increasing the expression of a target may be achieved by methods known in the art, such as, e.g., by transfecting (e.g., by electroporation, lipofection, etc.) or transducing (e.g., using a viral vector) a cell, tissue, organ or organism with a recombinant nucleic acid which encodes said target under the control of regulatory sequences effecting suitable expression level in said cell, tissue, organ or organism. By means of example and not limitation, the level of the target may be modulated via alteration of the formation of the target (such as, e.g., folding, or interactions leading to formation of a complex), and/or the stability (e.g., the propensity of complex constituents to associate to a complex or disassociate from a complex), degradation or cellular localisation, etc. of the target.

The term “antisense” generally refers to a molecule designed to interfere with gene expression and capable of specifically binding to an intended target nucleic acid sequence. Antisense agents typically encompass an oligonucleotide or oligonucleotide analogue capable of specifically hybridising to the target sequence, and may typically comprise, consist essentially of or consist of a nucleic acid sequence that is complementary or substantially complementary to a sequence within genomic DNA, hnRNA, mRNA or cDNA, preferably mRNA or cDNA corresponding to the target nucleic acid. Antisense agents suitable herein may typically be capable of hybridising to their respective target at high stringency conditions, and may hybridise specifically to the target under physiological conditions.

The term “ribozyme” generally refers to a nucleic acid molecule, preferably an oligonucleotide or oligonucleotide analogue, capable of catalytically cleaving a polynucleotide. Preferably, a “ribozyme” may be capable of cleaving mRNA of a given target protein, thereby reducing translation thereof. Exemplary ribozymes contemplated herein include, without limitation, hammer head type ribozymes, ribozymes of the hairpin type, delta type ribozymes, etc. For teaching on ribozymes and design thereof, see, e.g., U.S. Pat. No. 5,354,855, U.S. Pat. No. 5,591,610, Pierce et al. 1998 (Nucleic Acids Res 26: 5093-5101), Lieber et al. 1995 (Mol Cell Biol 15: 540-551), and Benseler et al. 1993 (J Am Chem Soc 115: 8483-8484).

“RNA interference” or “RNAi” technology is routine in the art, and suitable RNAi agents intended herein may include inter alia short interfering nucleic acids (siNA), short interfering RNA (siRNA), double-stranded RNA (dsRNA), micro-RNA (miRNA), and short hairpin RNA (shRNA) molecules as known in the art. For teaching on RNAi molecules and design thereof, see inter alia Elbashir et al. 2001 (Nature 411: 494-501), Reynolds et al. 2004 (Nat Biotechnol 22: 326-30), http://rnaidesigner.invitrogen.com/rnaiexpress, Wang & Mu 2004 (Bioinformatics 20: 1818-20), Yuan et al. 2004 (Nucleic Acids Res 32 (Web Server issue): W130-4), by M Sohail 2004 (“Gene Silencing by RNA Interference: Technology and Application”, 1^{st} ed., CRC, ISBN 0849321417), U Schepers 2005 (“RNA Interference in Practice: Principles, Basics, and Methods for Gene Silencing in C. elegans, Drosophila, and Mammals”, 1^{st} ed., Wiley-VCH, ISBN 3527310207), and D R Engelke & J J Rossi 2005 (“Methods in Enzymology, Volume 392: RNA Interference”, 1^{st} ed., Academic Press, ISBN 0121827976).

The various active substances of the present disclosure, such as inter alia complexes, proteins, nucleic acids, vectors, cells and agents as taught herein or pharmaceutically acceptable derivatives thereof, may be formulated into pharmaceutical compositions or formulations with one or more pharmaceutically acceptable carriers/excipients.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art.

Pharmaceutical compositions of the invention may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous, intramuscular, intraperitoneal or intrasternal injection or infusion), transdermal or transmucosal (such as, e.g., oral, sublingual, intranasal) administration, topical administration, rectal, vaginal or intra-tracheal instillation, and the like. In this way, the therapeutic effects attainable by the methods and compositions of the invention can be, for example, systemic, local, tissue-specific, etc., depending of the specific needs of a given application of the invention.

For example, for oral administration, pharmaceutical compositions may be formulated in the form of pills, tablets, lacquered tablets, coated (e.g., sugar-coated) tablets, granules, hard and soft gelatin capsules, aqueous, alcoholic or oily solutions, syrups, emulsions or suspensions. In an example, without limitation, preparation of oral dosage forms may be is suitably accomplished by uniformly and intimately blending together a suitable amount of the active compound in the form of a powder, optionally also including finely divided one or more solid carrier, and formulating the blend in a pill, tablet or a capsule. Exemplary but non-limiting solid carriers include calcium phosphate, magnesium stearate, talc, sugars (such as, e.g., glucose, mannose, lactose or sucrose), sugar alcohols (such as, e.g., mannitol), dextrin, starch, gelatin, cellulose, polyvinylpyrrolidine, low melting waxes and ion exchange resins. Compressed tablets containing the pharmaceutical composition can be prepared by uniformly and intimately mixing the active ingredient with a solid carrier such as described above to provide a mixture having the necessary compression properties, and then compacting the mixture in a suitable machine to the shape and size desired. Moulded tablets maybe made by moulding in a suitable machine, a mixture of powdered compound moistened with an inert liquid diluent. Suitable carriers for soft gelatin capsules and suppositories are, for example, fats, waxes, semisolid and liquid polyols, natural or hardened oils, etc.

For example, for oral or nasal aerosol or inhalation administration, pharmaceutical compositions may be formulated with illustrative carriers, such as, e.g., as in solution with saline, polyethylene glycol or glycols, DPPC, methylcellulose, or in mixture with powdered dispersing agents, further employing benzyl alcohol or other suitable preservatives, absorption promoters to enhance bioavailability, fluorocarbons, and/or other solubilising or dispersing agents known in the art. Suitable pharmaceutical formulations for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Illustratively, delivery may be by use of a single-use delivery device, a mist nebuliser, a breath-activated powder inhaler, an aerosol metered-dose inhaler (MDI) or any other of the numerous nebuliser delivery devices available in the art. Additionally, mist tents or direct administration through endotracheal tubes may also be used.

Examples of carriers for administration via mucosal surfaces depend upon the particular route, e.g., oral, sublingual, intranasal, etc. When administered orally, illustrative examples include pharmaceutical grades of mannitol, starch, lactose, magnesium stearate, sodium saccharide, cellulose, magnesium carbonate and the like, with mannitol being preferred. When administered intranasally, illustrative examples include polyethylene glycol, phospholipids, glycols and glycolipids, sucrose, and/or methylcellulose, powder suspensions with or without bulking agents such as lactose and preservatives such as benzalkonium chloride, EDTA. In a particularly illustrative embodiment, the phospholipid 1,2 dipalmitoyl-sn-glycero-3-phosphocholine (DPPC) is used as an isotonic aqueous carrier at about 0.01-0.2% for intranasal administration of the compound of the subject invention at a concentration of about 0.1 to 3.0 mg/ml.

For example, for parenteral administration, pharmaceutical compositions may be advantageously formulated as solutions, suspensions or emulsions with suitable solvents, diluents, solubilisers or emulsifiers, etc. Suitable solvents are, without limitation, water, physiological saline solution or alcohols, e.g. ethanol, propanol, glycerol, in addition also sugar solutions such as glucose, invert sugar, sucrose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. The compounds and pharmaceutically acceptable salts thereof of the invention can also be lyophilised and the lyophilisates obtained used, for example, for the production of injection or infusion preparations. For example, one illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

Where aqueous formulations are preferred, such may comprise one or more surfactants. For example, the composition can be in the form of a micellar dispersion comprising at least one suitable surfactant, e.g., a phospholipid surfactant. Illustrative examples of phospholipids include diacyl phosphatidyl glycerols, such as dimyristoyl phosphatidyl glycerol (DPMG), dipalmitoyl phosphatidyl glycerol (DPPG), and distearoyl phosphatidyl glycerol (DSPG), diacyl phosphatidyl cholines, such as dimyristoyl phosphatidylcholine (DPMC), dipalmitoyl phosphatidylcholine (DPPC), and distearoyl phosphatidylcholine (DSPC); diacyl phosphatidic acids, such as dimyristoyl phosphatidic acid (DPMA), dipahnitoyl phosphatidic acid (DPPA), and distearoyl phosphatidic acid (DSPA); and diacyl phosphatidyl ethanolamines such as dimyristoyl phosphatidyl ethanolamine (DPME), dipalmitoyl phosphatidyl ethanolamine (DPPE) and distearoyl phosphatidyl ethanolamine (DSPE). Typically, a surfactant:active substance molar ratio in an aqueous formulation will be from about 10:1 to about 1:10, more typically from about 5:1 to about 1:5, however any effective amount of surfactant may be used in an aqueous formulation to best suit the specific objectives of interest.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquidify and/or dissolve in the rectal cavity to release the drug.

Suitable carriers for microcapsules, implants or rods are, for example, copolymers of glycolic acid and lactic acid.

One skilled in this art will recognize that the above description is illustrative rather than exhaustive. Indeed, many additional formulations techniques and pharmaceutically-acceptable excipients and carrier solutions are well-known to those skilled in the art, as is the development of suitable dosing and treatment regimens for using the particular compositions described herein in a variety of treatment regimens.

The present active substances may be used alone or in combination with any anti-retroviral therapies known in the art (“combination therapy”). Combination therapies as contemplated herein may comprise the administration of at least one active substance of the present invention and at least one other pharmaceutically or biologically active ingredient. Said present active substance(s) and said pharmaceutically or biologically active ingredient(s) may be administered in either the same or different pharmaceutical formulation(s), simultaneously or sequentially in any order.

Exemplary anti-retroviral drugs in combination therapy with which the present active substances may be employed include, without limitation, nucleoside and nucleotide reverse transcriptase inhibitors, non-nucleoside reverse transcriptase inhibitors, protease inhibitors, integrase inhibitors, entry inhibitors, maturation inhibitors and broad spectrum inhibitors

The dosage or amount of the present active substances used, optionally in combination with one or more other active compound to be administered, depends on the individual case and is, as is customary, to be adapted to the individual circumstances to achieve an optimum effect. Thus, it depends on the nature and the severity of the disorder to be treated, and also on the sex, age, body weight, general health, diet, mode and time of administration, and individual responsiveness of the human or animal to be treated, on the route of administration, efficacy, metabolic stability and duration of action of the compounds used, on whether the therapy is acute or chronic or prophylactic, or on whether other active compounds are administered in addition to the agent(s) of the invention.

Without limitation, depending on the type and severity of the disease, a typical daily dosage might range from about 1 μg/kg to 100 mg/kg of body weight or more, depending on the factors mentioned above. For repeated administrations over several days or longer, depending on the condition, the treatment is sustained until a desired suppression of disease symptoms occurs. A preferred dosage of the active substance of the invention may be in the range from about 0.05 mg/kg to about 10 mg/kg of body weight. Thus, one or more doses of about 0.5 mg/kg, 2.0 mg/kg, 4.0 mg/kg or 10 mg/kg (or any combination thereof) may be administered to the patient. Such doses may be administered intermittently, e.g., every week or every two or three weeks.

Except when noted, “subject” or “patient” are used interchangeably and refer to animals, preferably warm-blooded animals, more preferably vertebrates, even more preferably mammals, still more preferably primates, and specifically includes human patients and non-human mammals and primates. Preferred patients are human subjects.

As used herein, a phrase such as “a subject in need of treatment” includes subjects that would benefit from treatment of a given condition, particularly of a retroviral infection. Such subjects may include, without limitation, those that have been diagnosed with said condition, those prone to contract or develop said condition and/or those in whom said condition is to be prevented.

The terms “treat” or “treatment” encompass both the therapeutic treatment of an already developed disease or condition, such as the therapy of an already developed retroviral infection, as well as prophylactic or preventative measures, wherein the aim is to prevent or lessen the chances of incidence of an undesired affliction, such as to prevent the chances of contraction and progression of a retroviral infection. Beneficial or desired clinical results may include, without limitation, alleviation of one or more symptoms or one or more biological markers, diminishment of extent of disease, stabilised (i.e., not worsening) state of disease, delay or slowing of disease progression, amelioration or palliation of the disease state, and the like. “Treatment” can also mean prolonging survival as compared to expected survival if not receiving treatment.

The term “prophylactically effective amount” refers to an amount of an active compound or pharmaceutical agent that inhibits or delays in a subject the onset of a disorder as being sought by a researcher, veterinarian, medical doctor or other clinician. The term “therapeutically effective amount” as used herein, refers to an amount of active compound or pharmaceutical agent that elicits the biological or medicinal response in a subject that is being sought by a researcher, veterinarian, medical doctor or other clinician, which may include inter alia alleviation of the symptoms of the disease or condition being treated. Methods are known in the art for determining therapeutically and prophylactically effective doses for the present compounds.

The terms “diagnosing” or “diagnosis” generally refer to the process or act of recognising, deciding on or concluding on a disease or condition in a subject on the basis of symptoms and signs and/or from results of various diagnostic procedures.

As used herein, “diagnosis of a disease or condition associated with a retrovirus” in a subject may particularly mean that the subject has said disease or condition, hence, is diagnosed as having said disease or condition. “Diagnosis of no disease or condition associated with a retrovirus” in a subject may particularly mean that the subject does not have said disease or condition, hence, is diagnosed as not having said disease or condition.

The terms “prognosticating” or “prognosis” generally refer to an anticipation on the progression of a disease or condition and the prospect (e.g., the probability, duration, and/or extent) of recovery.

A good prognosis may generally encompass anticipation of a satisfactory partial or complete recovery from a disease or condition, preferably within an acceptable time period. A good prognosis may more commonly encompass anticipation of not further worsening or aggravating of the disease or condition, preferably within a given time period.

A poor prognosis may generally encompass anticipation of a substandard recovery and/or unsatisfactorily slow recovery from, or substantially no recovery from or even further worsening of a disease or condition.

The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will contract said disease or condition, for example within a certain time period or by a certain age.

The present diagnostic methods may evaluate structure, activity and/or level of a complex or a host interactor protein as taught herein in a biological sample. In this context, the term structure may particularly encompass the primary (i.e., amino acid sequence), secondary, tertiary and quarternary structure of said complex or host interactor protein, including structural aspects due to one or more co- or post-expression modifications of said complex or host interactor protein as discussed elsewhere in this application.

A “deviation” of a first value from a second value may generally encompass any direction (e.g., increase: first value>second value; or decrease: first value<second value) and any extent of alteration.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD, or ±1×SE or ±2×SE). Deviation may also refer to a value falling outside of a reference range defined by values in a given population (for example, outside of a range which comprises 40%, 50%, 60%, 75% or 80% or 85% or 90% or 95% or even 100% of values in said population). In a further embodiment, a deviation may be concluded if an observed alteration is beyond a given threshold or cut-off. Such threshold or cut-off may be selected as generally known in the art to provide for a chosen sensitivity and/or specificity of the prediction, diagnosis and/or prognosis methods, e.g., sensitivity and/or specificity of at least 50%, or at least 60%, or at least 70%, or at least 80%, or at least 85%, or at least 90%, or at least 95%.

Reference values used in the present methods may be established according to known procedures previously employed for biomarkers. Such reference values may be established either within (i.e., constituting a step of) or external to (i.e., not constituting a step of) the present diagnostic, prognostic or predictive methods. Accordingly, any one of the methods taught herein may comprise a step of establishing a reference value for the structure, activity and/or level of a complex or host interactor protein, said reference value representing either (a) a prediction or diagnosis of no disease or condition associated with a retrovirus or a good prognosis for such disease or condition, or (b) a prediction or diagnosis of such disease or condition or a poor prognosis for such disease or condition.

Further provided is thus a method for establishing a reference value for the structure, activity and/or level of a complex or host interactor protein, said reference value representing:

(a) a prediction or diagnosis of no disease or condition associated with a retrovirus or a good prognosis for such disease or condition, or
(b) a prediction or diagnosis of such disease or condition or a poor prognosis for such disease or condition,
comprising:
(i) measuring the structure, activity and/or level of the complex or host interactor protein in:
(i a) one or more samples from one or more subjects not having such disease or condition or not being at risk of having such disease or condition or having a good prognosis for such disease or condition, or
(i b) one or more samples from one or more subjects having such disease or condition or being at risk of having such disease or condition or having a poor prognosis for such disease or condition, and
   (ii) storing the structure, activity and/or level of the complex or host interactor protein
(ii a) as measured in (i a) as the reference value representing the prediction or diagnosis of no such disease or condition or representing the good prognosis for such disease or condition, or
(ii b) as measured in (i b) as the reference value representing the prediction or diagnosis of such disease or condition or representing the poor prognosis for such disease or condition.

Interaction-querying techniques and particularly protein-protein interaction-querying techniques are commonly known in the art and may include inter alia yeast two-hybrid based methods, co-immunoprecipitation methods optionally in conjunction with mass spectrometry (MS) analysis methods, immunoassay technologies (such as among others direct ELISA, indirect ELISA, sandwich ELISA, competitive ELISA, multiplex ELISA, radioimmunoassay (RIA), ELISPOT technologies), affinity chromatography methods, etc. For guidance in this respect see inter alia P L Bartel & S Fields 1997 (“The Yeast Two-Hybrid System”, 1^{st} ed., Oxford University Press, ISBN 0195109384), H Fu 2004 (“Protein-Protein Interactions: Methods and Applications”, 1^{st} ed., Humana Press, ISBN 1588291200), N MacDonald 2001 (“Two-Hybrid Systems: Methods and Protocols”, 1^{st} ed., Humana Press, ISBN 0896038327), J M Walker 2005 (“The Proteomics Protocols Handbook”, 1^{st} ed., Humana Press, ISBN 1588295931) and J R Crowther 2000 (“The ELISA Guidebook”, 1^{st} ed., Humana Press, ISBN 0896037282).

It is apparent that there have been provided in accordance with the invention products, methods and uses that provide for substantial advantages as set forth above. While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The above aspects and embodiments are further supported by the following non-limiting examples.

### EXAMPLES

### Example 1

### Cloning of HIV-1, HIV-2, HTLV-1 and HTLV-2 ORFeomes

To clone HIV-1 and HIV-2 ORFs we used as PCR templates, the following DNA obtained through the AIDS Research and Reference Reagent Program, Division of AIDS, NIAID, NIH: pNL4-3 (Adachi et al. 1986. J Virol 59: 284-291); pCMV-rev (Lewis et al. 1990. J Virol 64: 1690-1697); pcDNA-Vphu and pcDNA-HVif (Nguyen et al. 2004. Virology 319: 163-175); Senegalese HIV-2 isolate (HIV-2/ST) (Kong et al. 1988. Science 240: 1525-1529); the 96ZM651.8 clone (Gao et al. 2003. AIDS Res Hum Retroviruses 19: 817-823); GST-Tat1 and GST-Tat2 (Rhim et al. 1994. J Acquir Immune Defic Syndr 7: 1116-1121).

To clone HTLV-1 and HTLV-2 ORFs, DNA clones MT-2 (Gray et al. 1990. Virology 177: 391-395), ATK (Seiki et al. 1983. Proc Natl Acad Sci USA 80: 3618-3622), pH6 B 3.5 and pH6 B 5.0 Chen et al. 1983. Nature 305: 502-505; Shimotohno et al. 1984. Proc Natl Acad Sci USA 81: 6657-6661) and pcDNA-SP1 (Cavanagh et al. 2006. Retrovirology 3: 15) were used as PCR templates to amplify individual ORFs. The specific primers for each ORF contained AttB1.1 and AttB2.1 gateway recombination sites forward 5′GGGGACAACTTTGTACAAAAAAGTTGGC (SEQ ID NO: 1) and reverse 5′GAGAGTTAGTGGCCCGCAGGTCGGGGGA (SEQ ID NO: 2) allowing recombinational cloning into the spectinomycin resistant donor vector pDONR223 by BP clonase (Invitrogen).

### Example 2

### LR Cloning into Yeast Two Hybrid and Mammalian Destination Expression Vectors

All full length and partial retroviral ORFs (rvORFs) were transferred by LR cloning into pDB-dest and pAD-dest-CYH (Vidalain et al. 2004. Methods 32: 363-370) to generate yeast expression vectors for DB-rvORF and AD-rvORF fusion proteins. For downstream functional assays, the human ORFs identified in yeast two-hybrid experiments were also subcloned from their corresponding entry clones into pDEST-Flag vectors.

### Example 3

### High-Throughput Yeast Two-Hybrid

AD-rvORF and DB-rvORF yeast expressing vectors were respectively transformed into MATa and MATα cells of two different yeast strains Mav103/203 and Y8800/8930. Transformed yeast cells were then spotted on solid synthetic complete (Sc) media lacking tryptophane (Sc-T) to select for AD-rvORF clones or leucine (Sc-L) for yeast containing DB-rvORF vectors. Growing colonies were cultured in liquid Sc-L or Sc-T media and stored in glycerol for subsequent use. All DB-ORFs in Mav103 strain or Y8930 were individually tested for auto-activation by growth on solid SC-L-H medium containing 20 mM (Mav103 strain) or 2 mM (Y8930 strain) of 3-amino-triazole (3-AT) to eliminate autoactivators baits that are able to activate reporter genes in the absence of AD plasmids. Aliquots of AD-rvORF transformed yeast were pooled to generate the AD-rvORF library.

Yeast two-hybrid screening was then performed as previously described (Rual et al. 2005. Nature 437: 1173-1178). Briefly, a 96-well format was used to mate each of 12,212 DB-ORFs MATα yeast strains of the human ORFeome version 3.1 (Lamesch et a/0.2007. Genomics 89: 307-315) with a pool of MATa yeast strains containing individual retroviral AD-rvORF. A reciprocal experiment was also performed by mating individual retroviral DB-rvORF yeast with the same 12,212 human AD-ORF pooled into 65 mini-libraries as previously described (Rual et al. 2005, supra). Diploid cells were selected on solid media Sc-L-T-H (containing 20 mM of 3-AT for the May strain), de novo autoactivators eliminated as described (Vidalain et al. 2004, supra). Consolidated colonies were re-grown on interaction selecting (Sc-L-T-H or Sc-L-T-A) and auto-activation control media to confirm interaction-specific transcriptional activation of histidine, adenine or (β-galactosidase reporter genes. Colonies finally were picked for PCR amplification and sequencing identification of the interacting AD- and DB-ORFs.

After sequence verification, we then transferred these retroviral ORFs (rvORFs) into the Y2H Gal4 activation domain (AD) vector, transformed MATa yeast strains and pooled individual yeast cells to generate the AD-rvORF library. As bait proteins, we used individual clones of the human ORFeome v 3.1 fused to the Gal4 DNA-binding domain (DB) and transformed into MATα yeast strain. In a reverse setup, we also tested by yeast mating in a 96-well format, each individual DB-rvORF against mini-libraries each containing a pool of 94 AD-human ORF clones. Yeast two-hybrid auto-activators, the main source of false positives in HT-Y2H data sets, were systematically removed by using the CHY2 counter-selectable marker, as previously described (Walhout & Vidal 1999. Genome Res 9: 1128-1134; Vidalain et al. 2004, supra).

### Example 4

### Pair-Wise Y2H Retest

Each human ORF found to interact with viral proteins was individually retested against all homologous proteins in the HIV/HTLV viruses. To this end, we performed a mating assay using MATα (May 203 or Y8930) and MATa (May 103 or Y8800) yeast cells containing individual DB and AD fused to interacting human and retroviral ORF, respectively. The resulting diploid cells then were tested for their ability to activate histidine and adenine or β-galactosidase reporter genes. False positives due to de novo autoactivation were again eliminated using the counter-selectable marker CYH2 as described (Rual et al. 2005, supra).

### Example 5

### Transactivation Assay

The plasmids pHIV1LTR-Luc or pHTLV1LTR-Luc containing a luciferase reporter gene under the control of the HIV-1 or HTLV-1 LTR promoters and plasmids expressing either HIV-1 Tat or HTLV-1 Tax and each human ORF found to interact with viral proteins were transfected into 10⁴ HEK293 cells using the calcium phosphate method. Twenty-four hours post-transfection, cells were washed three times with PBS, lysed and luciferase activities determined from two independent transfection experiments in triplicate. We computed a paired t-test to assess the difference of the means between samples with and without the human interactor. For a trial to be considered positive, the relative luciferase activities has to be >=2 or <=0.5, and the p-value of the t-test<0.05.

### Example 6

### KEGG Pathways Analysis

Pathways definitions were uploaded from the KEGG database (September 2008). We used Fisher's exact test to determine the pathways enrichment of direct targets of viral proteins. The significance of indirect targets enrichment was evaluated through a randomization process. Precisely, we ran 200000 simulations where we randomized the identity of the direct targets. The interactors of these targets were identified in the unbiased PPI network (Rual et al. 2005, supra), interactors belonging to each pathway were counted and the resulting distribution was compared to the observed counts from our experiments. We computed an empirical False Discovery Rate (FDR) to determine the significance of the enrichment. The FDR is defined here as the proportion of random trials giving at least the observed number of indirect targets in the considered pathway. The FDR was corrected for multiple testing using bonferroni correction (corrected FDR—FDR Corr). Pathways with a FDR Corr<0.05 and at least four observed proteins were considered as significant.

We used the CCSB-HI1 network (Rual et al. 2005, supra) to compute the enrichment of indirect targets for KEGG pathways to avoid human bias (some proteins have been more studied than others) that would prejudice the results with a network coming from literature curation. On the other hand, the plotted networks have been built from a literature curated interactions (LCI) network, to show information as complete as possible. The LCI network was built as the union of Human protein-protein interactions from the BIND (Bader et al. 2003. Nucleic Acids Res 31: 248-250, DIP (Xenarios et al. 2002. Nucleic Acids Res 30: 303-305, HPRD (Mishra et al. 2006. Nucleic Acids Res 34: D411-414, INTACT (Kerrien et al. 2007. Nucleic Acids Res 35: D561-565) and MINT (Chatr-aryamontri et al. 2007. Nucleic Acids Res 35: D572-574).

To construct sub-networks for each pathway, direct targets of viral proteins belonging to the pathway, and direct targets linked to two or more viral proteins were selected as “seeds”. Interactors of these seeds in the human-human LCI network and belonging to the considered pathway were then selected as indirect targets, and all interactions between seeds and indirects targets were plotted, along with our virus-human PPI network. All network figures were constructed using Cytoscape (Yeung et al. 2008. Curr Protoc Bioinformatics, Chapter 8:Unit 8.13).

### Example 7

### Identification of HIV/HTLV—Human Interactions

Using Gateway-based ORFeome libraries for most HIV-1, HIV-2, HTLV-1 and HTLV-2 ORFs (see Table 1) in a Y2H screen against the Human ORFeome v 3.1, we identified 1440 positive diploid colonies representing 336 potential interactions between human proteins and HIV or HTLV viral proteins (FIG. 1). All these interactions were then independently verified with a pair-wise Y2H assay. Several HIV and HTLV structural and regulatory proteins have significant sequence or functional similarity. These homologous viral proteins might share one or more interacting partners amongst the human proteins, but may fail to be identified in the initial screen because (i) highly overlapping or similar viral ORFs may be misidentified with BLAST, (ii) given the pooling technique used, some interactions can be missed in a single screen (Venkatesan et al. 2009. Nat Methods 6: 83-90; Braun et al. 2009. Nat Methods 6: 91-97; Simonis et al. 2009. Nat Methods 6: 47-54). We retested all homologous HIV and HTLV proteins for interaction with each human ORF found in our initial screen with at least one homologous viral protein. For instance, all human ORFs identified as HIV-1 Gag interactors were also retested against HIV-2, HTLV-1 and HTLV-2 Gag and Pol proteins. This strategy combines the advantages of the pooling technique and individual testing, to reduce the cost and workload of the initial screen while keeping the ability to differentiate very similar proteins, overcome sensibility and specificity issues and compare negative results. The final data set contained 212 interactions between 19 viral proteins and 131 human proteins (Table 1, FIG. 2). Among the 212 PPIs we identified in our screen, 28, 26, 87 and 71 interactions involved HIV-1, HIV-2, HTLV-1 and HTLV-2-encoded proteins, respectively.

### Example 8

### Transactivation Assay

Many HIV/HTLV-human interactions in our data set (130/212) involved the retroviral transactivator proteins HTLV-1 Tax (57/212), HTLV-2 Tax2 (49/212), HIV-1 Tat (10/212) and HIV-2 Tat (14/212). Human proteins interacting with viral transactivators likely influence Tat or Tax transactivation. Expression vectors encoding human ORFs and HIV-1 Tat or HTLV-1 Tax proteins were tested in a transactivation reporter assay against reporter plasmids harboring HIV-1 or HTLV-1 viral promoter sequences cloned upstream of the luciferase reporter gene. In the controls Tat or Tax respectively activated HIV or HTLV LTR-directed gene expression. Co-transfection of interacting human ORFs identified 71 proteins (54% of the 131 human targets in our data set) that regulated HIV (26.7% of targets), HTLV (37.4% of targets) or both (9.9% of targets) LTR promoter activation by Tat or Tax (see Tables 7 and 8; Table 13 below containing quantitative data on the regulation of transactivation). Wherever not expressly specified, constructs used herein were generally made by sub-cloning entry clones from the ORFeome V3.1 into suitable destination vectors.

TABLE 13
Regulation of HIV or HTLV LTR transactivation by host interactors;
quantitative data (fold change).

| Human gene | HIV_LTR | HTLV_LTR |
|---|---|---|
| symbol | transactiv. | transactiv. |
| BHLHB2 | — | 0.20 |
| C10orf3 | 2.79 | 0.41 |
| C16orf33 | 2.50 | — |
| C1orf94 | — | 0.41 |
| C20orf141 | 0.35 | 2.80 |
| C8orf32 | — | 0.26 |
| CCDC24 | — | 2.88 |
| CRSP9 | — | 2.89 |
| CRX | 0.24 | — |
| DAZAP2 | — | 3.31 |
| DGCR6L | — | 0.24 |
| DHRS10 | — | 0.41 |
| DIPA | — | 0.28 |
| DLAT | 6.90 | — |
| DLX2 | 0.24 | 0.30 |
| DVL2 | 0.40 | — |
| FANCG | 0.27 | 3.48 |
| FATE1 | — | 0.49 |
| FLJ10726 | — | 0.19 |
| FLJ22471 | — | 2.25 |
| FTH1 | — | 0.17 |
| FXR2 | — | 0.38 |
| GADD45GIP1 | — | 0.42 |
| GCC1 | 3.78 | 0.29 |
| GOPC | — | 3.13 |
| HOXA3 | 0.29 | 0.25 |
| HOXB9 | — | 3.69 |
| HOXD3 | 7.30 | — |
| HSFY1 | — | 0.43 |
| KCTD1 | — | 0.42 |
| KIAA1949 | — | 0.35 |
| KIF9 | — | 0.37 |
| KLC3 | — | 0.31 |
| KRT15 | 0.33 | — |
| KRT8 | 0.19 | 0.24 |
| LENG1 | — | 0.34 |
| LNX2 | 0.26 | 0.28 |
| LOC391257 | 0.20 | 0.27 |
| LOC541468 | 2.38 | — |
| LOC91661 | 0.16 | — |
| LZTS2 | 0.49 | 0.23 |
| MAD1L1 | — | 0.20 |
| MINA | 0.40 | — |
| MIZF | 2.50 | — |
| MLLT11 | 3.41 | — |
| MRPS6 | — | 0.38 |
| MYEF2 | — | 2.53 |
| MYST2 | — | 3.66 |
| NEFL | — | 0.45 |
| NIF3L1 | — | 0.39 |
| NKAP | 2.68 | — |
| NOS3 | — | 0.45 |
| OTX2 | — | 7.37 |
| PCBP1 | — | 0.44 |
| PDE9A | 0.28 | — |
| PIAS2 | 0.42 | — |
| PSMF1 | — | 14.83 |
| RNPS1 | — | 11.12 |
| SF3A3 | 0.49 | — |
| SP100 | 3.69 | — |
| SPAG5 | 0.16 | — |
| SPG21 | — | 0.21 |
| TEX11 | 4.41 | — |
| TFIP11 | 0.19 | 0.45 |
| TRAF2 | 0.22 | — |
| TRIP6 | 2.87 | — |
| TSC22D4 | 5.43 | 3.75 |
| TTC23 | — | 0.21 |
| UBE2I | 3.34 | — |
| ZMAT1 | 0.20 | — |
| ZNF614 | 0.28 | 5.24 |

There were 28 host factors that significantly enhanced Tat or Tax transactivation activities (14 for HIV LTR, 15 for HTLV LTR) suggesting their potential implication in viral replication and persistence in infected cells. Interestingly, TSC22D4, a member of TSC22 domain family of leucine zipper transcriptional regulators, was the only protein in our data set able to up-regulate both Tat and Tax transactivation activities. Another group of cellular proteins (48/71, 21 for HIV LTR and 34 for HTLV LTR) down-regulated viral promoters activation and may be implicated in viral latency allowing HIV and HTLV viruses to escape the immune surveillance or in coordinating distinct phases of the viruses cycles.

### Example 9

### Comparison of the HIV and HTLV Interactome Maps

Our standardized experimental conditions, combining high-throughput Y2H with a defined search space and with systematic retesting of homologous proteins, allows comparisons between interacting protein pairs. Network representations of our data allowed identification of shared and distinct PPI within each genus of pathogenic retroviruses and between HIV and HTLV species (see Table 1, FIG. 2). A comparison between the interactions involving HTLV-1 Tax and HTLV-2 Tax identified 34 human proteins that bind HTLV-1 Tax protein but not with the HTLV-2 homolog. Similarly, 26 of the HTLV-2 Tax interactors did not interact with HTLV-1 Tax (see Table 1, FIG. 2). Considering differential oncogenic potential of the two HTLV viruses (Feuer & Green 2005. Oncogene 24: 5996-6004) and the central roles of their Tax proteins, these PPI observations could shed light on mechanisms of cellular transformation by the Tax oncoprotein. Also identified are nine human proteins involved in PPIs with at least one HIV and one HTLV encoded proteins (see Tables 1, 6, FIG. 2). The nine common interactors were the cell cycle regulator CDC23, two potential E3 ubiquitin ligases (TRAF2 and LNX2), two BTB/POZ domain-containing proteins (KCTD1 and KLHL12) that may serve as substrate-specific adapters for the CUL3-based ubiquitin-protein E3 ligase complexes (Bayon et al. 2008. FEBS J 275: 3900-3910; Rondou et al. 2008. J Biol Chem 283: 11083-11096), three homeobox transcription factors (HOXD3, DLX2 and CRX), and a cytoskeletal component (KRT6A).

### Example 10

### Enrichment of Viral Targets for Biological Pathways

We tested if human targets of viral proteins were enriched for annotated pathways in the Kyoto Encyclopedia of Genes and Genomes (KEGG) (Kanehisa et al. 2008. Nucleic Acids Res 36: D480-484). We observed no significant enrichment of any KEGG pathway for direct first-degree targets. We also analyzed second-degree interactors, those human proteins that interact with viral targets in the human-human PPI network (Rual et al. 2005, supra). Proteins associated with Ubiquitin mediated proteolysis and apoptosis pathways and proteins involved in a number of human cancers were overrepresented compared to random expectation (Table 14). The particular second-degree interactors, their corresponding first-degree host interactor proteins, and their attribution to specific cellular pathways in humans is detailed in Table 15. We also found enrichment for proteins from the Notch signaling pathway and Huntington disease proteins as indirect targets for HIV and HTLV proteins.

TABLE 14
Organisation of second-degree interactors of host interactor proteins
disclosed herein into cellular pathways.

| | Pathway | | | Odds | | |
|---|---|---|---|---|---|---|
| Pathway Name | ID | Observed | Random | Ratio | FDR | FDR Corr |
| Apoptosis | hsa04210 | 7 | 1.92 | 3.65 | 4.5E−05 | 6.8E−03 |
| Chronic myeloid | hsa05220 | 8 | 1.74 | 4.60 | 2.5E−05 | 3.8E−03 |
| leukaemia | | | | | | |
| Colorectal cancer | hsa05210 | 7 | 2.29 | 3.06 | 1.6E−04 | 2.4E−02 |
| ErbB signalling pathway | hsa04012 | 7 | 1.82 | 3.84 | 1.5E−04 | 2.3E−02 |
| Glioma | hsa05214 | 7 | 0.83 | 8.48 | 1.4E−04 | 2.0E−02 |
| Huntington's disease | hsa05040 | 6 | 0.37 | 16.04 | <2.5E−06 | <3.8E−04 |
| Insulin signalling pathway | hsa04910 | 11 | 2.25 | 4.88 | 7.0E−05 | 1.1E−02 |
| Long-term potentiation | hsa04720 | 6 | 0.49 | 12.22 | 9.0E−05 | 1.4E−02 |
| Lysine degradation | hsa00310 | 4 | 0.92 | 4.35 | 3.0E−05 | 4.5E−03 |
| Melanoma | hsa05218 | 4 | 0.70 | 5.70 | 1.6E−04 | 2.3E−02 |
| Notch signalling pathway | hsa04330 | 4 | 1.93 | 2.07 | <2.5E−06 | <3.8E−04 |
| Olfactory transduction | hsa04740 | 4 | 0.17 | 24.04 | <2.5E−06 | <3.8E−04 |
| Prostate cancer | hsa05215 | 6 | 1.14 | 5.25 | 1.5E−05 | 2.3E−03 |
| Small cell lung cancer | hsa05222 | 8 | 2.33 | 3.43 | 2.2E−04 | 3.2E−02 |
| Ubiquitin mediated | hsa04120 | 12 | 3.28 | 3.66 | 5.0E−06 | 7.5E−04 |
| proteolysis | | | | | | |

For each enriched KEGG pathway, we show the pathway identifier in the KEGG database (Pathway ID), the number of observed proteins belonging to the considered pathway (Observed), the number of proteins in the pathway expected at random (Random), the ratio between the number of observed proteins and the expected number (Odds Ratio), the false discovery rate (FDR), and the corrected FDR (FDR corr.).

TABLE 15
Second-degree interactor proteins, their corresponding first-degree interactors, their
attribution to specific cellular pathways in humans. Pubmed ID refers to publications
evidencing the involvement of the respective proteins in said pathways.

| | HIV or HTLV host | 2^{nd} degree | |
|---|---|---|---|
| Pathway | interactor | interactor | Pubmed ID |
| Apoptosis | Dlc2 | BIRC4 | 16189514 |
| | EWSR1 | BAD | 16189514 |
| | | DFFA | 16189514 |
| | FATE1 | TRAF2 | 16189514 |
| | FLJ32855 | TRAF2 | 16189514 |
| | HGS | PIK3R1 | 10970851 |
| | LZTS2 | TRAF2 | 16189514 |
| | MIZF | TRAF2 | 16189514 |
| | NIF3L1 | TRAF2 | 16189514 |
| | NUDT18 | TRAF2 | 16189514 |
| | RIBC2 | TRAF2 | 16189514 |
| | SPG21 | TRAF2 | 16189514 |
| | TRAF2 | BIRC2 | 8643514; 9384571; 11907583; |
| | | | 16189514, 16282325 |
| | | BIRC3 | 8643514; 11907583; 16282325 |
| | | CASP8 | 11340079; 12887920; 16227629 |
| | | CHUK | 11359906 |
| | | IKBKB | 11359906 |
| | | TNF | 12887920 |
| | | TNFRSF1A | 8565075; 8943045 |
| | | TRAF2 | 8069916; 8702708; 10206649; |
| | | | 10518213; 14743216; 16189514 |
| | | TRADD | 8565075; 8702708; 10892748; |
| | | | 14743216 |
| | | RIPK1 | 8612133; 8702708 |
| | | FADD | 16227629 |
| | | CFLAR | 9208847 |
| | | MAP3K14 | 9020361; 9275204; 11278268 |
| Chronic myeloid | DIPA | CDKN1A | 16189514 |
| leukemia | EWSR1 | BAD | 16189514 |
| | | SMAD4 | 16189514 |
| | HGS | SMAD3 | 11094085 |
| | | PIK3R1 | 10970851 |
| | KLHL12 | ARAF | 16189514 |
| | MINA | MYC | 12091391 |
| | TRAF2 | CDKN1B | 16189514 |
| | | CHUK | 11359906 |
| | | IKBKB | 11359906 |
| Colorectal cancer | DVL2 | DVL2 | 16189514 |
| | | RAC1 | 12533515 |
| | | TP53 | 16189514; 16713569 |
| | | AXIN1 | 10829020 |
| | | FZD4 | 12958364 |
| | EWSR1 | BAD | 16189514 |
| | | SMAD4 | 16189514 |
| | FLJ32855 | DVL2 | 16189514 |
| | HGS | EGFR | 12953068 |
| | | SMAD2 | 11094085 |
| | | SMAD3 | 11094085 |
| | | MET | 8380735 |
| | | PIK3R1 | 10970851 |
| | KLHL12 | ARAF | 16189514 |
| | | DVL3 | 16189514 |
| | KRT6A | EGFR | 15657067 |
| | MINA | MYC | 12091391 |
| | TRAF2 | DVL2 | 16189514 |
| | | MAPK9 | 10490605 |
| | | APPL | 16189514 |
| ErbB signaling pathway | DIPA | CDKN1A | 16189514 |
| | EWSR1 | BAD | 16189514 |
| | | PRKCA | 9341188 |
| | HGS | EGFR | 12953068 |
| | | PAK1 | 11397816 |
| | | PIK3R1 | 10970851 |
| | KLHL12 | ARAF | 16189514 |
| | KRT6A | EGFR | 15657067 |
| | LZTS2 | PAK7 | 16189514 |
| | MINA | MYC | 12091391 |
| | TRAF2 | CDKN1B | 16189514 |
| | | FRAP1 | 14743216 |
| | | MAPK9 | 10490605 |
| Glioma | DIPA | CDKN1A | 16189514 |
| | EWSR1 | CALM1 | 9341188 |
| | | PRKCA | 9341188 |
| | HGS | EGFR | 12953068 |
| | | PIK3R1 | 10970851 |
| | KLHL12 | ARAF | 16189514 |
| | KRT6A | EGFR | 15657067 |
| | TRAF2 | FRAP1 | 14743216 |
| Huntington's disease | EWSR1 | CALM1 | 9341188 |
| | | CREBBP | 12459554 |
| | HGS | CLTC | 11532964 |
| | | HAP1 | 12021262 |
| | SS18L1 | CREBBP | 14716005 |
| | | EP300 | 14716005 |
| | TRAF2 | CASP8 | 11340079; 12887920; 16227629 |
| | | CLTC | 14743216 |
| Insulin signaling | DIPA | EXOC7 | 16189514 |
| pathway | EWSR1 | BAD | 16189514 |
| | | CALM1 | 9341188 |
| | | FASN | 16189514 |
| | HGS | PIK3R1 | 10970851 |
| | | EXOC7 | 16189514 |
| | KLHL12 | ARAF | 16189514 |
| | SLC2A4 | AKT2 | 11500317 |
| | | PRKAR1A | 16396496 |
| | TRAF2 | FRAP1 | 14743216 |
| | | IKBKB | 11359906 |
| | | MAPK9 | 10490605 |
| Long-term potentiation | EWSR1 | CALM1 | 9341188 |
| | | CREBBP | 12459554 |
| | | PRKCA | 9341188 |
| | KLHL12 | ARAF | 16189514 |
| | LDOC1 | ATF4 | 16189514 |
| | SS18L1 | CREBBP | 14716005 |
| | | EP300 | 14716005 |
| Lysine degradation | BHLHB2 | SETDB1 | 16169070 |
| | DIPA | PLOD3 | 16189514 |
| | EWSR1 | SUV39H2 | 16189514 |
| | FLJ32855 | EHMT2 | 16189514 |
| | HGS | EHMT2 | 16189514 |
| | TRAF2 | AASDHPPT | 16189514 |
| Melanoma | DIPA | CDKN1A | 16189514 |
| | EWSR1 | BAD | 16189514 |
| | HGS | EGFR | 12953068 |
| | | MET | 8380735 |
| | | PIK3R1 | 10970851 |
| | KLHL12 | ARAF | 16189514 |
| | KRT6A | EGFR | 15657067 |
| Notch signaling pathway | DVL2 | DVL2 | 16189514 |
| | EWSR1 | CREBBP | 12459554 |
| | FLJ32855 | DVL2 | 16189514 |
| | KLHL12 | DVL3 | 16189514 |
| | LNX2 | NUMB | 11782429; 11922143 |
| | SS18L1 | CREBBP | 14716005 |
| | | EP300 | 14716005 |
| | TRAF2 | DVL2 | 16189514 |
| Olfactory transduction | EWSR1 | CALM1 | 9341188 |
| Prostate cancer | DIPA | CDKN1A | 16189514 |
| | EWSR1 | BAD | 16189514 |
| | | CREBBP | 12459554 |
| | HGS | EGFR | 12953068 |
| | | PIK3R1 | 10970851 |
| | KLHL12 | ARAF | 16189514 |
| | KRT6A | EGFR | 15657067 |
| | LDOC1 | ATF4 | 16189514 |
| | SS18L1 | CREBBP | 14716005 |
| | | EP300 | 14716005 |
| | TRAF2 | CDKN1B | 16189514 |
| | | CHUK | 11359906 |
| | | FRAP1 | 14743216 |
| | | GSTP1 | 16636664 |
| | | IKBKB | 11359906 |
| Small cell lung cancer | Dlc2 | BIRC4 | 16189514 |
| | FATE1 | TRAF2 | 16189514 |
| | FLJ32855 | TRAF2 | 16189514 |
| | HGS | PIK3R1 | 10970851 |
| | LZTS2 | TRAF2 | 16189514 |
| | MINA | MYC | 12091391 |
| | MIZF | TRAF2 | 16189514 |
| | NIF3L1 | TRAF2 | 16189514 |
| | NOS3 | AKT1 | 10376603; 11696579 |
| | | AKT2 | 11988487 |
| | | NOS3 | 9516116; 10074942; 12437348 |
| | NUDT18 | TRAF2 | 16189514 |
| | NUP62 | TRAF3 | 10781837 |
| | PIAS2 | PIAS1 | 11877418 |
| | RIBC2 | TRAF2 | 16189514 |
| | SPG21 | TRAF2 | 16189514 |
| | TRAF2 | BIRC2 | 8643514; 9384571; 11907583; |
| | | | 16189514; 16282325 |
| | | BIRC3 | 8643514; 11907583; 16282325 |
| | | CDKN1B | 16189514 |
| | | CHUK | 11359906 |
| | | IKBKB | 11359906 |
| | | TRAF1 | 8069916; 8702708; 14743216 |
| | | TRAF2 | 8069916; 8702708; 10206649; |
| | | | 10518213; 14743216; 16189514 |
| | | TRAF6 | 16189514 |
| Ubiquitin mediated | BACH1 | BRCA1 | 11301010; 11877378 |
| proteolysis | BHLHB2 | UBE2I | 11278694 |
| | CDC23 | CDC27 | 16648845 |
| | Dlc2 | BIRC4 | 16189514 |
| | EWSR1 | TRIM37 | 16189514 |
| | | WWP1 | 16189514 |
| | | WWP2 | 16189514 |
| | HGS | NEDD4 | 12230472 |
| | | UBE2I | 16189514 |
| | NUDT18 | TRIM37 | 16189514 |
| | PDE9A | TRIM32 | 16189514 |
| | PIAS2 | PIAS1 | 11877418 |
| | RIBC2 | TRIM37 | 16189514 |
| | TRAF2 | BIRC2 | 8643514; 9384571; 11907583; |
| | | | 16189514; 16282325 |
| | | BIRC3 | 8643514; 11907583; 16282325 |
| | | MAP3K1 | 16636664 |
| | | TRIM37 | 11279055 |
| | | TRAF6 | 16189514 |
| | UBE2I | PIAS1 | 12177000; 14609633 |
| | | PIAS2 | 12177000; 16189514 |
| | | UBA2 | 16455490 |

### Notch Signalling Pathway

The highly conserved Notch signalling pathway regulates diverse cell fate decisions, including differentiation, proliferation, communication and specification. We found that members of the Notch signalling pathway, including Numb, disheveled (Dvl) proteins, cAMP-response element-binding protein (CREB)-binding protein (CREBBP or CBP) and p300, are targeted by HIV (Tat, Nef and Gag) and HTLV (Tax, Rex, Hbz and Gag) proteins (FIG. 3).

In example 18 we show that inhibition of the Notch pathway can reduce retroviral (in particular, HLTV-1) expression. On the other hand, activation of the Notch pathway may counteract retroviral latency.

### The Apoptosis Pathway

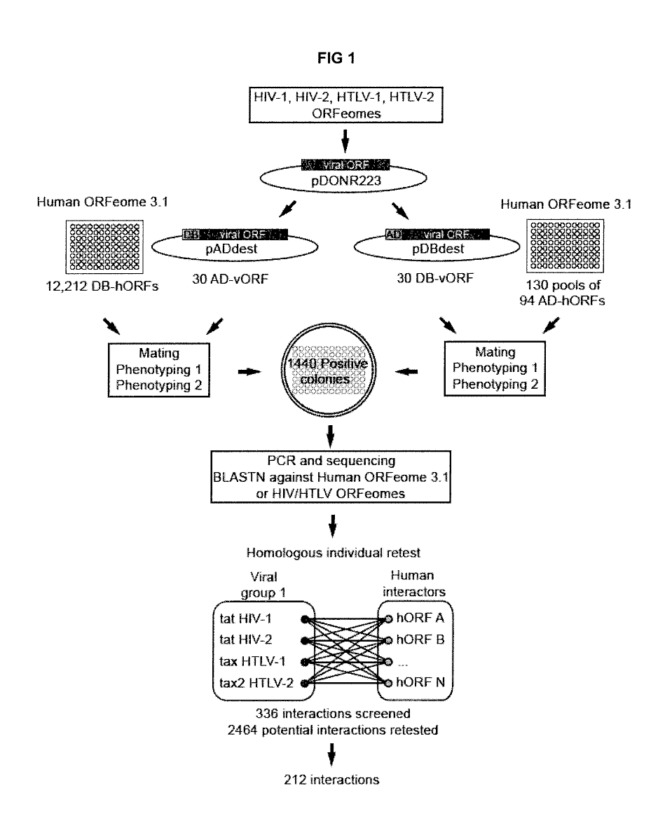

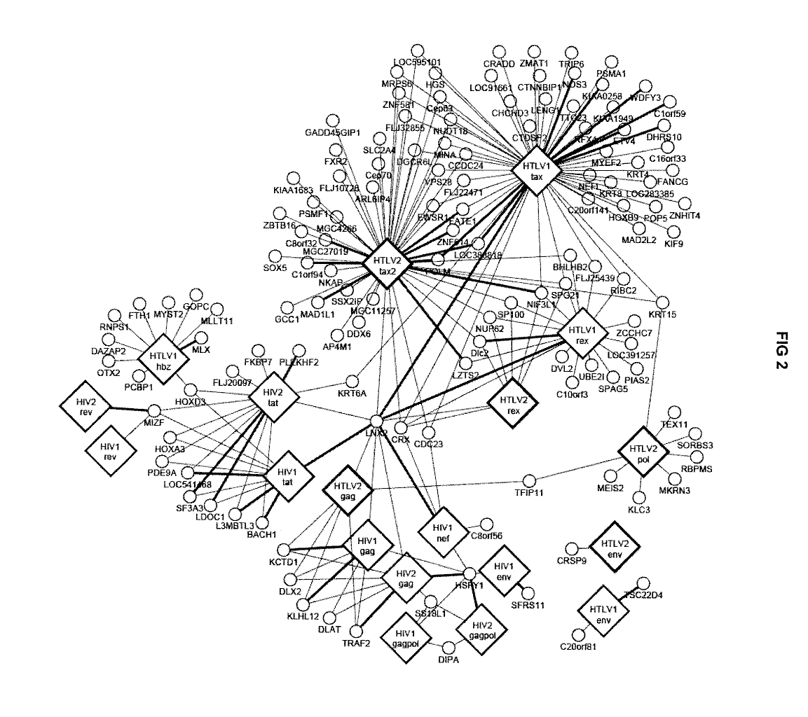

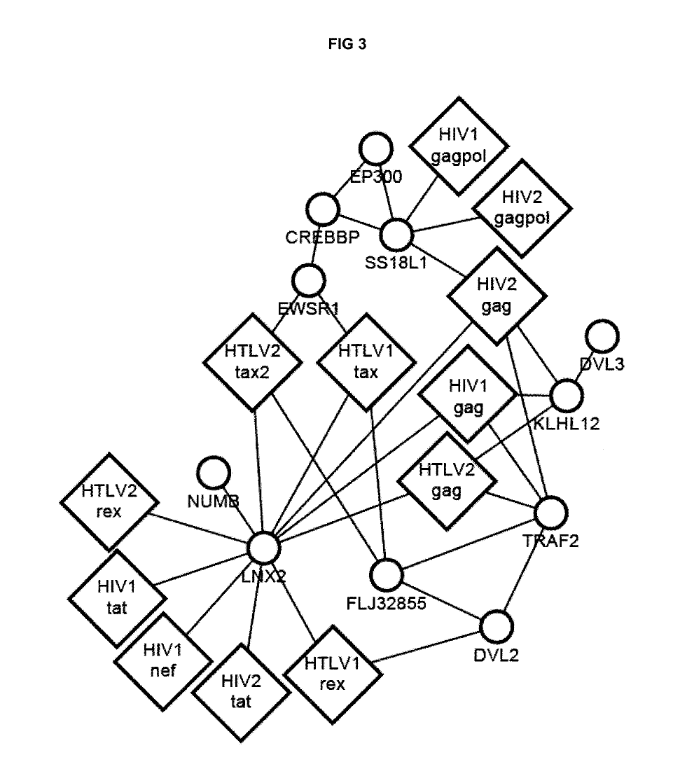

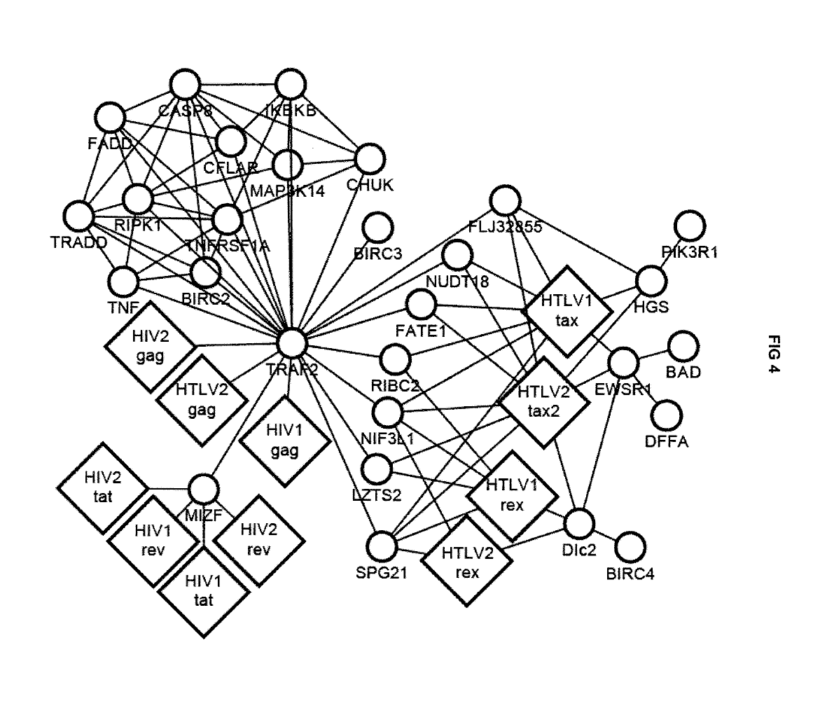

Graphical representation of the apoptotic pathway sub-network allowed us to identify the tumor necrosis factor (TNF) receptor-associated factor type 2 (TRAF-2) as a central node mediating interactions between HIV/HTLV proteins, the TNF receptor (TNFR) signalling and the Akt/PI3K survival pathway (FIG. 4). Depending on its interacting partners, TRAF2 signals drive contradictory cellular responses. Direct binding to the cytoplasmic domain of TNFR2, which does not contain a death domain, can trigger NFkB and JNK activation, but TRAF2 also indirectly mediates the signal from a death domain containing receptors such as TNFR1 via interaction with FADD and TRADD pro-caspases adaptor factors (Hsu et al. 1996. Cell 84: 299-308). We found that retroviral Gag proteins bind TRAF2, directly connecting these viral proteins to the extrinsic TNFR apoptotic pathway. We also found that TRAF2 is a second-degree target of HIV Tat and Rev proteins via a transcription repressor, the methyl-CpG binding domain (MBD) type 2 zinc finger (MIZF). The network representation also shows that HTLV Tax and Rex proteins are more closely linked to the Akt/PI3K and mitochondrial apoptotic pathways than HIV proteins. Indeed, we identified interactions between HTLV Tax and HGS and EWSR1 and KEGG analysis indicated that phosphatidylinositol-3-kinase (PI3K) BCL2-antagonist of cell death (Bad), and DNA fragmentation factor alpha (DFFA) proteins are second-degree targets. (FIG. 4). We also found that the HTLV Rex proteins interact with DLC2 (for deleted in liver cancer 2), a tumor growth suppressor that binds to the inhibitor of apoptosis type 4 (IAP4 also called BIRC4), and so may play an important anti-apoptotic role by controlling caspase activities in HTLV infected cells.

### Ubiquitin Mediated Proteolysis

We identified interaction of HTLV-1 Tax with the core subunit of the proteasome PSMA1 and the interaction of HTLV-2 Tax2 with the regulator subunit of the proteasome PSMF1.

Moreover, we identified additional cellular E3 ubiquitin ligases (LNX2 and TRAF2) which directly interact with retroviral proteins and which may play important roles in induced perturbations of the proteasomal pathway. Both proteins contain a RING finger domain, a type of domain that has been shown to simultaneously bind ubiquitination enzymes and their substrates and hence acts as ligase (Lorick et al. 1999. Proc Natl Acad Sci USA 96: 11364-11369; Joazeiro & Weissman 2000. Cell 102: 549-552).

In addition to the RING domain, LNX2 contains four PDZ domains, which are conserved protein modules commonly found in proteins that function as scaffolds for signaling complexes. Since binding to PDZ domains is crucial for the oncogenic potential of different human tumor viruses, including HTLV-1 (Javier 2008. Oncogene 27: 7031-7046), LNX2 could be an important E3 ubiquitin ligase toward HIV and HTLV proteins. In addition to its role in TNF-mediated c-Jun N-terminal kinase and NF-kB activation (Lee et al. 1997. Immunity 7: 703-713; Yeh et al. 1997. Immunity 7: 715-725), TRAF2 also possesses a potential E3 ubiquitin ligase activity through its N-terminal RING domain. TRAF2 is, in turn, targeted for proteasomal degradation by another E3 ubiquitin ligase, the inhibitor of apoptosis (c-IAP1) (Li et al. 2002. Nature 416: 345-347). The interaction with retroviral Gag proteins we have shown could unbalance cell death and survival responses by either promoting TRAF2 proteasomal degradation or by contributing to its ubiquitin ligase activity.

Example 11 shows that the interaction of TRAF2 with retroviral Gag proteins promotes TRAF2 proteasomal degradation, particularly through the RING domain. Mass spectrometry analysis of TRAF2 interactors in living cells also revealed disruption of several known TRAF2 cellular interactions in the presence of HIV-1 Gag, which could, without wishing to be bound to any theory, lead to perturbation of the proteasomal degradation pathway and unbalance cell death and survival responses through apoptotic pathways.

Also, example 17 demonstrates that silencing LNX2 inhibits Tat transactivation and viral infection, suggesting a hypothesis that LNX2 could be an important E3 ubiquitin ligase toward HIV and HTLV proteins.

### Example 11

### TRAF 2 is a HIV-1 Gag Cellular Target

The following evidences that TRAF-2 represents a functionally relevant target of HIV-1 Gag.

The TRAF2 complex expressed in cultured cells was purified by immonoprecipitation using an anti-TRAF antibody and the immunoprecipitates were loaded on a 10% (w/v) polyacrylamide-SDS gel, after electrophoresis the gel was stained with colloidal Coomassie (Fermentas, Lituania). The bands of interest were cut out and digested with trypsin. Peptides were analyzed by capillary LC-tandem mass spectrometry in a LTQ XL ion trap mass spectrometer (ThermoScientific, San Jose, Calif.) fitted with a microelectrosprayprobe. The data were analyzed with the ProteomeDiscoverer software (ThermoScientific), and the proteins were identified with SEQUEST against a target-decoy non redundant human protein database obtained from NCBI. The false discovery rate was below 5%.

Table 16 contains identified proteins in each sample. “+” and “−” indicates presence or absence of a given protein in the control, the TRAF2 or TRAF2+ HIV-1 Gag samples.

TABLE 16

| | | | | TRAF2 |
|---|---|---|---|---|
| Score | Description | Control | TRAF2 | HIV-1 Gag |
| 19.71 | heterogeneous nuclear ribonucleoprotein K isoform | − | − | + |
| | b [Homo sapiens] | | | |
| 194.54 | myosin, heavy polypeptide 9, non-muscle [Homo | + | + | + |
| | sapiens] (PMID: 16982613) | | | |
| 42.91 | splicing factor 3b, subunit 1 isoform 1 [Homo | − | + | − |
| | sapiens] | | | |
| 28.25 | OTU domain containing 4 protein isoform 1 [Homo | − | + | − |
| | sapiens] (PMID: 15258597) | | | |
| 57.33 | heat shock protein 90 kDa alpha (cytosolic), class A | − | + | − |
| | member 1 isoform 2 [Homo sapiens] (PMID: | | | |
| | 12939259) | | | |
| 47.58 | gelsolin isoform b [Homo sapiens] PMID: | − | + | − |
| | 10521462) | | | |
| 51.85 | heat shock 90 kDa protein 1, beta [Homo sapiens] | − | + | − |
| 11.33 | nucleoporin 93 kDa [Homo sapiens] | − | + | − |
| 13.40 | RIO kinase 1 isoform 2 [Homo sapiens] | − | + | − |
| 27.92 | karyopherin beta 1 [Homo sapiens] | − | + | − |
| 79.06 | eukaryotic translation initiation factor 4B [Homo | − | + | − |
| | sapiens] | | | |
| 69.70 | unc-84 homolog B [Homo sapiens] | − | + | − |
| 15.82 | SCY1-like 2 protein [Homo sapiens] | − | + | − |
| 305.07 | actin, gamma 1 propeptide [Homo sapiens] (PMID: | − | + | − |
| | 10617615) | | | |
| 163.46 | alpha 1 actin precursor [Homo sapiens] | − | + | − |
| 83.17 | methylosome protein 50 [Homo sapiens] | − | + | − |
| 20.52 | heterogeneous nuclear ribonucleoprotein H1 [Homo | − | + | − |
| | sapiens] | | | |

Interestingly, interaction with HIV1-Gag results in disruption of the TRAF2 complex, as is also apparent from comparison of the middle and right lanes of FIG. 5.

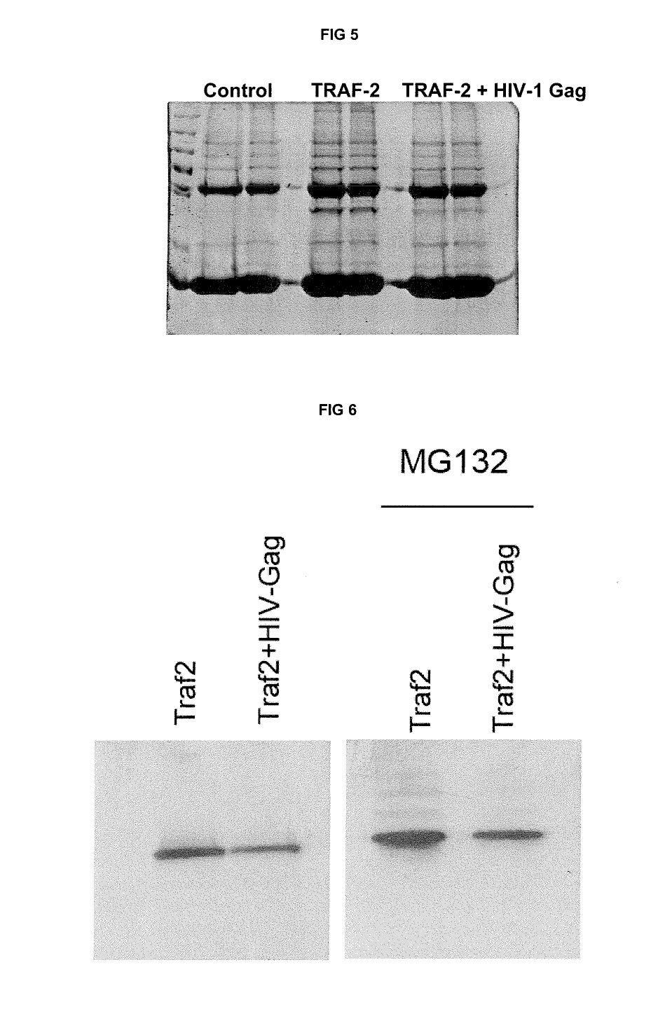

Further, as shown in FIG. 6, the presence of HIV1-Gag induces degradation of TRAF2, and this degradation is not inhibited by the proteasomal inhibitor MG132.

To further corroborate that HIV Gag protein targets TRAF2 for proteasomal degradation, we first co-expressed in HEK293 cells HIV-1 Gag and TRAF2 or its truncation mutants lacking the TRAF domain (ΔTRAF) required for upstream signaling, or lacking the RING domain (ΔRING) required for E3 ubiquitin ligase function and downstream signaling.

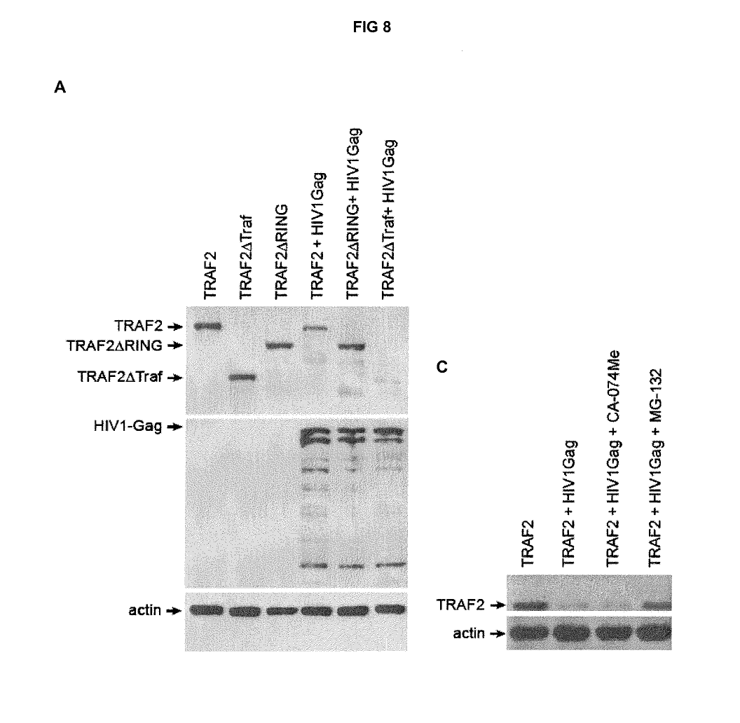

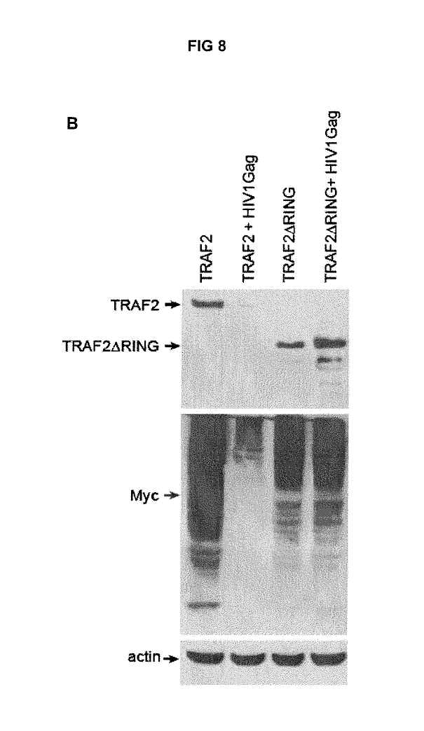

HEK293T cells were cultured in a humidified atmosphere with 5% CO2 at 37° C. in DMEM supplement with 10% of fetal bovine serum and antibiotics. HEK293T cells were transfected using the calcium phosphate method as previously described (Twizere et al. Blood, 2007, vol. 109, 1051-1060). In some cases (FIG. 8), HEK293T cells were pretreated with the cathepsin B inhibitor CA-074Me (10 μM) or the proteasomal inhibitor MG-132 (1 μM) for 24 H; washed in ice-cold PBS and lysed in IPLS buffer (1% NP-40, 10% glycerol, 120 mM NaCl, 20 mM Tris pH 7.5, 2 mM EDTA, and complete protease inhibitor cocktail (Roche). Expression constructs for glutathione-S-transferase (GST) fusion proteins of human TRAF2 and truncation mutants lacking N-terminal RING finger or C-terminus TRAF domains were provided by Dr. Chadee (Korchnak et al. Cell Signal, 2009, vol. 21, 1620-1625). P55 GAG/GFP construct is from NIH AIDS reagent (Sandefur et al. J Virol, 1998, vol. 72, p. 2723-2732).

In the presence of HIV-1 Gag we observed reduction of TRAF2 and ΔTRAF protein levels but not with the RING domain truncated mutant (FIG. 8A). To test whether TRAF2-E3 ubiquitin ligase activity was affected by HIV1 Gag, we next co-transfected a Myc-Ubiquitin expressing vector and evaluated the levels of Myc-tagged cellular ubiquitylated proteins by immunoblotting. As shown on FIG. 8B, degradation of TRAF2 correlated with a reduction of Myc-ubiquitylated proteins. Finally, the degradation of TRAF2 could be blocked by pre-incubating cells with the proteasome inhibitor MG132 (FIG. 8C). Together these data indicate that HIV-1 Gag induces proteasomal degradation of TRAF2 through its RING domain.

Without wishing to be bound by any theory, finding that retroviral Gag proteins directly bind TRAF2 links the Gag proteins to the TNF receptor (TNFR) signaling apoptotic pathway and identifies TRAF2 as a crucial protein that potentially mediates differential deregulation of apoptotic pathways by HIV and HTLV proteins. In particular, previous reports indicated that stimulation through TNFR2 induced TRAF2 ubiquitination, subsequent proteasomal degradation (Zhao et al. J Biol Chem, 2007, vol. 282, 7777-7782) and sensitivity to TNFα-induced cell death (Vince et al. J Cell Biol, 2008, vol. 182, 171-184). Retroviral infection is frequently associated with elevated TNFα and HIV patients with treatment failure have persistent activation of the TNF system components (Aukrust et al. J Infect Dis, 1999, vol. 179, 74-82). Consequently, we hypothesize that HIV Gag protein may target TRAF2 for proteasomal degradation, thereby facilitating sensitivity to TNFα-induced cell death.

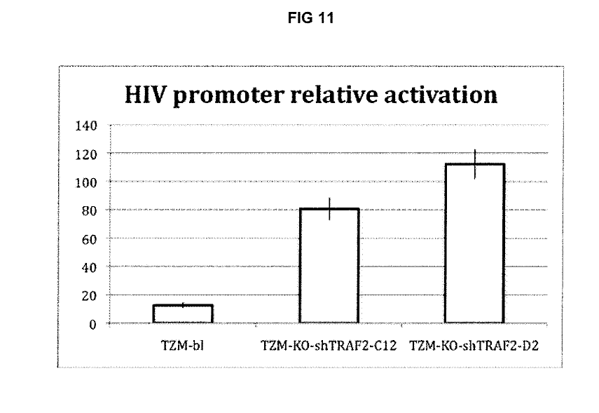

Corroborating the above expectation, knock-out of TRAF2 in a cellular system increased HIV LTR promoter activation in retroviral infection indicator cell lines (TZM-bl cells) (see FIG. 11).

### Example 12

### Knock-Down of HIV or HTLV Interactors

The present host interactors are, each individually or in combination of two or more interactors, knocked-down in cultured cells using standard RNAi mediated downregulation of gene expression. The knock-down of said interactors is shown to affect the characteristics of the infection of said cells by HIV and/or HTLV.

All interactors as identified herein (see inter alia Tables 1-10) are tested in the knock-down assay; particularly preferred are:
the common HIV/HTLV interactors: CDC23, TRAF2, LNX2, KCTD1, KLHL12, HOXD3, DLX2, CRX and KRT6A; or
host interactors which modulate transactivation of HIV or HTLV LTR, more preferably interactors as identified in Tables 7, 8, 13, even more preferably those modulating both HIV and HTLV LTR transactivation: TSC22D4, HOXA3, LNX2, DLX2, LZTS2, LOC391257, KRT8, TFIP11, SPAG5, SF3A3, FLJ10726, MAD1L1, SPG21; or
host interactors which have a potential E3 ubiquitin ligase activity (LNX2 and TRAF2) or a potential adaptor domain to E3 ubiquitin ligase activity (KCTD1 and KLHL12); or
TRAF2.

### Example 13

### Over-Expression of HIV or HTLV Interactors

The present host interactors are, each individually or in combination of two or more interactors, over-expressed in cultured cells using standard transient or stable gene expression techniques, e.g., transfecting or transducing the cells with an expression vector carrying the respective gene(s). The over-expression of said interactors is shown to affect the characteristics of the infection of said cells by HIV and/or HTLV.

All interactors as identified herein (see inter alia Tables 1-10) are tested in the over-expression assay; particularly preferred are:
the common HIV/HTLV interactors: CDC23, TRAF2, LNX2, KCTD1, KLHL12, HOXD3, DLX2, CRX and KRT6A; or
host interactors which modulate transactivation of HIV or HTLV LTR, more preferably interactors as identified in Tables 7, 8, 13, even more preferably those modulating both HIV and HTLV LTR transactivation: TSC22D4, HOXA3, LNX2, DLX2, LZTS2, LOC391257, KRT8, TFIP11, SPAG5, SF3A3, FLJ10726, MAD1L1, SPG21; or
host interactors which have a potential E3 ubiquitin ligase activity (LNX2 and TRAF2) or a potential adaptor domain to E3 ubiquitin ligase activity (KCTD1 and KLHL12); or
TRAF2.

### Example 14

### Identification of Peptides and Chemicals Disrupting the Interactions

Complexes including the present host interactors and their respective HIV or HTLV interaction counterparts are reconstituted in vitro or achieved by co-expression in cultured cells. Said complexes or cells are exposed to peptides or chemical compounds from a custom or commercially available library. The effect of the peptides or chemical compounds on the complex formation (particularly activity, level or stability) is monitored by immunoassay, immunofluorescence, Alpha screen, immunoprecipitation or yeast two hybrid methods. Peptides and chemical compounds that disrupt the complexes are selected and are found to affect the characteristics of infection of cultured cells by HIV and/or HTLV.

All interactors as identified herein (see inter alia Tables 1-10) are tested in the screening assay; particularly preferred are:
the common HIV/HTLV interactors: CDC23, TRAF2, LNX2, KCTD1, KLHL12, HOXD3, DLX2, CRX and KRT6A; or
host interactors which modulate transactivation of HIV or HTLV LTR, more preferably interactors as identified in Tables 7, 8, 13, even more preferably those modulating both HIV and HTLV LTR transactivation: TSC22D4, HOXA3, LNX2, DLX2, LZTS2, LOC391257, KRT8, TFIP11, SPAG5, SF3A3, FLJ10726, MAD1L1, SPG21; or
host interactors which have a potential E3 ubiquitin ligase activity (LNX2 and TRAF2) or a potential adaptor domain to E3 ubiquitin ligase activity (KCTD1 and KLHL12); or
TRAF2.

### Example 14

### Dose Response Experiment Using Host Interactors that Modulate Transactivation of HIV or HTLV LTR

Transactivation assay as in Example 5 is used. Varying quantities of expression plasmids encoding the present host interactors, each individually or in combination of two or more interactors, are introduced to the cells and the transactivation of HIV or HTLV LTR is monitored.

Expression of interactors which affect the transactivation of HIV or HTLV LTR is shown to affect the characteristics of the infection of cultured cells by HIV and/or HTLV.

Particularly preferred interactors for these dose response experiments are as defined above (see inter alia Tables 7, 8, 13). For example, particularly preferred are the interactors shown herein to modulate both HIV and HTLV LTR transactivation: TSC22D4, HOXA3, LNX2, DLX2, LZTS2, LOC391257, KRT8, TFIP11, SPAG5, SF3A3, FLJ10726, MAD1L1, SPG21.

### Example 15

### Co-Regulation Experiments Using Host Interactors that Modulate Transactivation of HIV or HTLV LTR

Transactivation assay as in Example 5 is used. Expression plasmids encoding the present host interactors, each individually or in combination of two or more interactors, are introduced to the cells. The cells are also exposed to one or more cellular cytokines (TNF, IFN, IL-2), histone deacetylase inhibitors, proteasomal inhibitors and NF-kB inhibitors. The transactivation of HIV or HTLV LTR is monitored.

### Example 16

### Effect of TRAF2 Peptides on HIV or HTLV Infection

TRAF2 is over-expressed in HIV or HTLV infected cells, or TRAF2 derived peptides are introduced to HIV or HTLV infected cells, or peptides or chemicals inhibiting TRAF2 are screened, identified and introduced to HIV or HTLV infected cells. These agents are shown to affect the characteristics of the infection of said cells by HIV and/or HTLV, in particular shown to inhibit apoptosis (particularly by HIV) and/or transformation (particularly by HTLV).

### Example 17

### Functional Assessment of LNX

LNX2 is a host interactor and target of particular interest, interacting with several viral proteins in the present assays and down-regulating both HIV and HTLV LTR promoters in vitro. To further demonstrate the involvement of LNX2 in HIV-1 infection, we used a HIV-1 indicator cell line (TZM-bl), which express endogenous CXCR4 and stably transfected CD4 and CCR5 receptors. TZM-bl cells also contain integrated copies of the luciferase and β-galactosidase (n-Gal) genes under the control of a HIV-1 promoter (Platt et al. J Virol, 1998, vol. 72, 2855-2864).

TZM-bl cells were cultured in a humidified atmosphere with 5% CO2 at 37° C. in DMEM supplement with 10% of fetal bovine serum and antibiotics. TZM-bl cells were obtained from NIH AIDS Research & Reference Reagent Program and transfected using TransIT®-LT1 reagent according to the manufacturer instructions (Mirus-Bio).

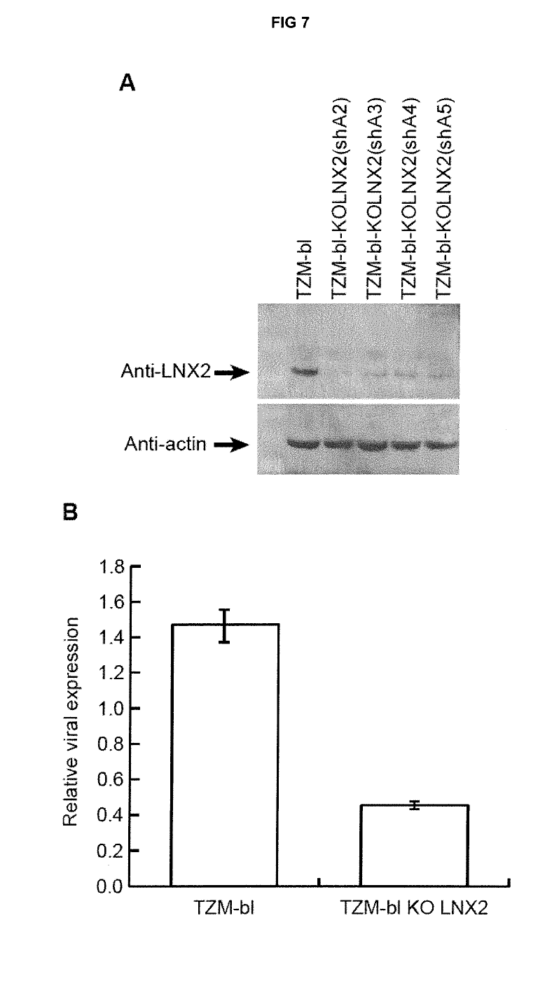

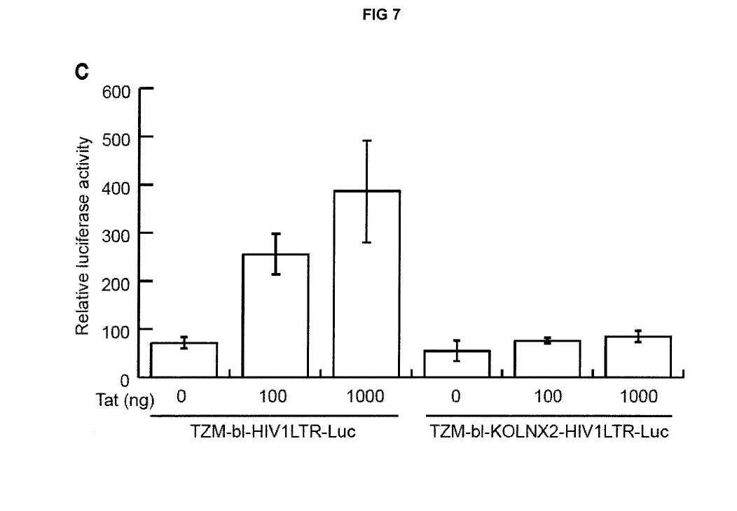

Viral particles expressing shRNA targeting various sequences of the LNX2 mRNA (Root et al. Nat Methods, 2006, vol. 3, 715-719) were prepared as described and TZM-bl infected cells were selected using puromycin (Tiscornia et al. Nat Protoc, 2006, vol. 1, 241-245). An aliquot of each cell line was then used in a western blot experiment using an anti-LNX2 antibody (Santa Cruz Biotechnology, Inc.) to test for KO efficiency. We generated 4 different TZM-bl cell lines stably expressing short hairpins RNA (shRNA) against LNX2 (FIG. 7A).

TZM-bl cells stably expressing a shRNA for LNX2 (TZM-bl-KO-shLNX2) and control cells were cultured for 24 hours and infected with the X4-tropic HIV-1NL4.3 viral strain (MOI, 5) for additional 24 hours. Beta-galactosidase activities were measured using a β-Gal Reporter Gene Assay kit according the manufacturer instructions (Roche). Differences of expression were assessed with one-tailed Student's t-test on triplicate experiments. LNX2 depletion significantly decreased viral infection (p<0.0035) (FIG. 7B).

TZM-bl cells stably expressing a shRNA for LNX2 (TZM-bl-KO-shLNX2) and control cells were cultured for 24 hours and transfected with increasing amounts (10, 100 and 1000 ng) of a HIV1 Tat expressing construct and luciferase activities measured as previously described (Twizere et al. Blood, 2007, vol. 109, 1051-1060). Differences of expression were assessed with one-tailed Student's t-test on triplicate experiments. LNX2 depletion significantly inhibited Tat transactivation activity in TZM-bl cells with medium (p<0.03) and high (p<0.05) HIV-1-promoter induction (FIG. 7C).

### Example 18

### Involvement of Notch Pathway in Retroviral Infection

HTLV-1 transformed cell line (MT4) from Dr. Douglas Richman (Harada et al. Science, 1985, col. 229, p. 563-566) was obtained through the AIDS Research and Reference Reagent Program, Division of AIDS, NIAID, NIH. MT4 cells were cultured in RPMI supplemented with 10% fetal bovine serum and antibiotics. MT-4 cells were treated for 48 hours with a γ-secretase inhibitor (L-685,458) (Shearman et al. Biochemistry, 2000, vol. 39, 8698-8704). at 1 μM or vehicle (0.5% DMSO). Total RNA were then isolated by Trizol method, subjected to Dnase treatment and cDNAs synthesized using the RevertAid First Strand cDNA Synthesis kit according to the manufacturer instructions (Fermentas). Quantitative Real-time PCR for GAPDH, HBZ, Gag and Tax expression was performed on StepOne instrument (Applied Biosystem) using SYBR green dye (Eurogentec). Viral mRNA expression data are calculated relative to GAPDH mRNA expression data as 2̂(CT(GAPDH)—CT(HBZ/Gag/Tax)) over three times triplicate experiments for each gene, and differences were assessed through one-tailed Student's t-test.

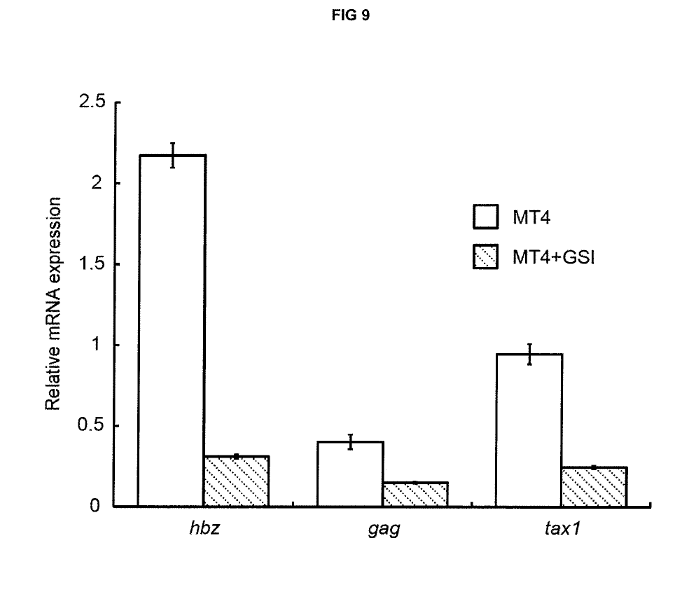

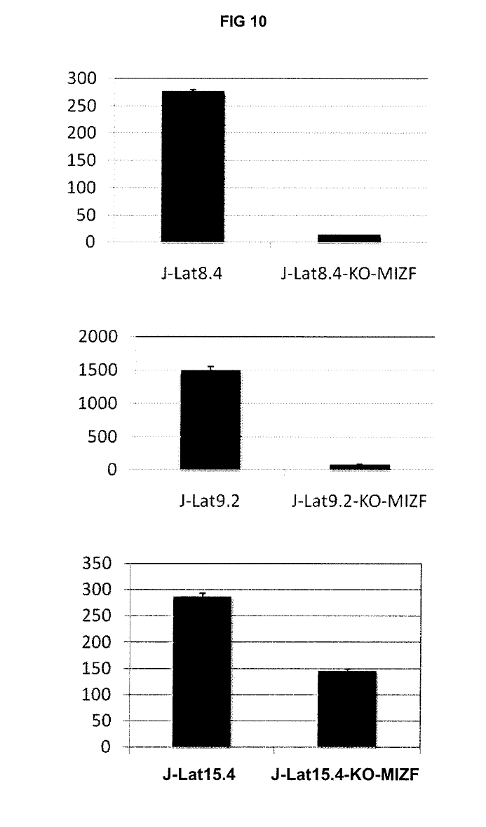

To directly assess the involvement of the Notch pathway in viral infection, we treated HTLV-1 transformed cell line (MT4) with a γ-secretase inhibitor (GSI) (L-685,458) and tested whether inhibition of the Notch pathway could influence HTLV-1 expression in MT4 cell line. Surprisingly, we showed by quantitative RT-PCR, that inhibition of the Notch pathway significantly lowers HTLV-1 HBZ (p<2.1E-5), Gag (p<0.04) and Tax1 (p<0.003) expression in MT4 cells (FIG. 9), suggesting that GSI could be a new class of retroviral replication inhibitors.

### Example 19

### Functional Assessment of MIZF

J-lat cells are different clones isolated after infection of Jurkat T cells with a HIV virus. These clones are latently infected as they express the virus upon treatment with Tumor necrosis factor alpha (TNF-α) or trichostatin A (TSA) (Kauder et al. Plos Pathogens, 2005; vol 5, issue 6, e1000495). MIZF knock down in J-lat cell lines was done by using small hairpin RNA targeting MIZF mRNA. Cells were selected using puromycin and treated for 24 hours with TSA (500 nM) and TNF (10 ng/ml). Cells were then collected by centrifugation and HIV p24 protein expression measured by enzyme-linked immunosorbent assays (ELISA). Cell viability was determined by Roche's cell proliferation reagent WST-1. Values are the relative means of HIV p24 from tree independent experiments in triplicate.

HIV persists in resting latent cells of infected persons under active antiretroviral therapy. Reactivation of latent HIV by targeting proteins involved in HIV latency and persistence could allow clearance of latently infected cells. Our data shows that knock down of MIZF inhibits reactivation of latently-infected cells and establish MIZF as a key factor in viral persistence. MIZF is involved in G1/S transition of cell cycle, histone deacytylase genes expression and may bind to methylated HIV DNA through methyl-CpG binding domain protein 2 (MBD2).

### Example 20

### Functional Assessment of TSC22D4

Expression of TSC22D4 in TZM-bl HIV indicator cells and in Jurkat T cell lines is eliminated using an RNAi approach. The resulting TZM-bl and Jurkat KO-TSC22D4 cells are infected with HIV. TSC22D4 knock-out cells show markedly altered HIV viral expression, thus establishing TSC22D4 as a key factor and target in controlling HIV infection.

Expression of TSC22D4 in Jurkat T cell lines is eliminated using an RNAi approach. The resulting Jurkat KO-TSC22D4 cells are infected with HTLV. TSC22D4 knock-out cells show markedly altered HTLV viral expression, thus establishing TSC22D4 as a key factor and target in controlling HTLV infection.

Agents modulating TSC22D4 are identified using screening assays as described herein. Said agents represent lead compounds having the potential as a general inhibitor of retroviral gene expression.

1. An isolated complex comprising a first and second proteins, wherein:
the first protein is a protein of a retrovirus, preferably a protein of a human retrovirus including human pathogenic and non-pathogenic retrovirus, more preferably a protein of the human immunodeficiency virus (HIV) or of the human T-lymphotropic virus (HTLV), or a functional fragment or variant of such a protein; and
the second protein is selected from TRAF2, LNX2, MIZF, TSC22D4, AP4M1, ARL61P4, BACH1, BHLHB2, C10orf3, C16orf33, Clorf59, Clorf94, C20orf141, C20orf81, C8orf32, C8orf56, CCDC24, Cep63, Cep70, CHCHD3, CRADD, CRSP9, CRX, CTDSP2, CTNNBIP1, DAZAP2, DDX6, DGCR6L, DHRS10, DIPA, DLAT, D1c2, DLX2, DVL2, ETV4, EWSR1, FANCG, FATE1, FKBP7, FLJ10726, FLJ20097, FLJ22471, FLJ25439, FLJ32855, FTH1, FXR2, GADD45GIP1, GCC1, GOPC, HGS, HOXA3, HOXB9, HOXD3, HSFY1, KCTD1, KIAA0258, KIAA1683, KIAA1949, KIF9, KLC3, KLHL12, KRT15, KRT4, KRT6A, L3 MBTL3, LDOC1, LENG1, LOC283385, LOC388818, LOC391257, LOC541468, LOC595101, LOC91661, LZTS2, MAD2L2, MEIS2, MGC11257, MGC27019, MGC4266, MINA, MKRN3, MLLT11, MLX, MRPS6, MYEF2, MYST2, NEFL, NIF3L1, NKAP, NOS3, NUDT18, OTX2, PCBP1, PDE9A, PIAS2, PLEKHF2, POLM, POP5, RBPMS, RFX4, RIBC2, RNPS1, SF3A3, SFRS11, SLC2A4, SORBS3, SOX5, SP100, SPAG5, SPG21, SS18L1, SSX21P, TEX11, TFIP11, TRIP6, TTC23, VPS28, WDFY3, ZBTB16, ZCCHC7, ZMAT1, ZNF581, ZNF614, ZNHIT4 or a functional fragment or variant of any one thereof.

2. An isolated complex comprising a first and second proteins, wherein:
the first protein is a protein of HIV or a functional fragment or variant of an HIV protein, and the second protein is selected from TRAF2, LNX2, MIZF, CDC23, CRX, DLX2, HOXD3, KCTD1, KLHL12, KRT6A or a functional fragment or variant of any one thereof; or
the first protein is a protein of HTLV or a functional fragment or variant of an HTLV protein, and the second protein is selected from TRAF2, LNX2, TSC22D4, CRX, DLX2, HOXD3, KCTD1, KLHL12, KRT6A, or a functional fragment or variant of any one thereof.

3. The isolated complex according to claim 1, wherein the second protein is selected from TRAF2, LNX2, MIZF, TSC22D4, DLX2, FLJ10726, HOXA3, LOC391257, LZTS2, SF3A3, SPAG5, SPG21, TFIP11 or a functional fragment or variant of any one thereof.

4. The isolated complex according to claim 1, wherein the second protein is TRAF2, LNX2, MIZF or TSC22D4 or a functional fragment or variant thereof.

5. An isolated nucleic acid encoding the complex of any one of claims 1 to 4.

6. A vector comprising the nucleic acid of claim 5, preferably wherein said vector is an expression vector.

7. A host cell comprising the isolated complex according to any one of claims 1 to 4, the isolated nucleic acid of claim 5 or the vector of claim 6.

8. A host organism comprising the isolated complex according to any one of claims 1 to 4, the isolated nucleic acid of claim 5, the vector of claim 6 or the host cell of claim 7.

9. A composition or formulation comprising the isolated complex according to any one of claims 1 to 4, the isolated nucleic acid of claim 5, the vector of claim 6, the host cell of claim 7 or the host organism of claim 8, and one or more additional components, preferably wherein the composition or formulation is a pharmaceutical composition or formulation and said one or more additional components are one or more pharmaceutically acceptable carriers.

10. A kit of parts comprising the isolated complex according to any one of claims 1 to 4, the isolated nucleic acid of claim 5, the vector of claim 6, the host cell of claim 7, the host organism of claim 8 or the composition or formulation of claim 9.

11. The isolated complex according to any one of claims 1 to 4, the isolated nucleic acid of claim 5, the vector of claim 6, the host cell of claim 7, the host organism of claim 8 or the composition or formulation of claim 9, for use for use as a medicament, preferably for use in the treatment of a disease or condition associated with a retrovirus, preferably wherein the disease or condition associated with a retrovirus is a disease or condition associated with a human retrovirus including human pathogenic and non-pathogenic retrovirus, more preferably HIV or HTLV.

12. A complex-binding agent capable of specifically binding to the complex according to any one of claims 1 to 4.

13. A method for selecting the complex-binding agent as defined in claim 12 comprising: (a) providing one or more, preferably a plurality of, test complex-binding agents; (b) selecting from the test complex-binding agents of (a) those which bind to the complex; and (c) counter-selecting from the test complex-binding agents selected in (b) those which bind to any one or more individual constituents of the complex, preferably those which bind to at least the first and/or second proteins of the complex, more preferably those which bind to at least the first and second proteins of the complex.

14. A complex-modulating agent capable of modulating the activity and/or level of the complex according to any one of claims 1 to 4.

15. A method for selecting the complex-modulating agent as defined in claim 14 comprising: (a) providing one or more, preferably a plurality of, test complex-modulating agents; and (b) selecting from the test complex-modulating agents of (a) those which modulate the activity and/or level of the complex.

16. A method for selecting, from one or more and preferably a plurality of test agents, a candidate therapeutic agent useful in the treatment of a disease or condition associated with a retrovirus, preferably a human retrovirus including human pathogenic and non-pathogenic retrovirus, more preferably HIV or HTLV, comprising the method steps of any one of claim 13 or 15.

17. A host interactor-modulating agent capable of modulating the activity and/or level of any one or more host interactor proteins selected from TRAF2, LNX2, MIZF, TSC22D4, AP4M1, ARL61P4, BACH1, BHLHB2, C10orf3, C16orf33, Clorf59, Clorf94, C20orf141, C20orf81, C8orf32, C8orf56, CCDC24, Cep63, Cep70, CHCHD3, CRADD, CRSP9, CRX, CTDSP2, CTNNBIP1, DAZAP2, DDX6, DGCR6L, DHRS10, DIPA, DLAT, D1c2, DLX2, DVL2, ETV4, EWSR1, FANCG, FATE1, FKBP7, FLJ10726, FLJ20097, FLJ22471, FLJ25439, FLJ32855, FTH1, FXR2, GADD45GIP1, GCC1, GOPC, HGS, HOXA3, HOXB9, HOXD3, HSFY1, KCTD1, KIAA0258, KIAA1683, KIAA1949, KIF9, KLC3, KLHL12, KRT15, KRT4, KRT6A, L3 MBTL3, LDOC1, LENG1, LOC283385, LOC388818, LOC391257, LOC541468, LOC595101, LOC91661, LZTS2, MAD2L2, MEIS2, MGC11257, MGC27019, MGC4266, MINA, MKRN3, MLLT11, MLX, MRPS6, MYEF2, MYST2, NEFL, NIF3L1, NKAP, NOS3, NUDT18, OTX2, PCBP1, PDE9A, PIAS2, PLEKHF2, POLM, POPS, RBPMS, RFX4, RIBC2, RNPS1, SF3A3, SFRS11, SLC2A4, SORBS3, SOX5, SP100, SPAG5, SPG21, SS18L1, SSX21P, TEX11, TFIP11, TRIP6, TTC23, VPS28, WDFY3, ZBTB16, ZCCHC7, ZMAT1, ZNF581, ZNF614, ZNHIT4 or a functional fragment or variant of any one thereof.

18. A method for selecting the host interactor-modulating agent according to claim 17 comprising: (a) providing one or more, preferably a plurality of, test host interactor-modulating agents; and (b) selecting from the test host interactor-modulating agents of (a) those which modulate the activity and/or level of the one or more host interactor proteins.

19. A method for selecting, from one or more and preferably a plurality of test agents, a candidate therapeutic agent useful in the treatment of a disease or condition associated with a retrovirus, preferably a human retrovirus including human pathogenic and non-pathogenic retrovirus, more preferably HIV or HTLV, comprising the method steps of claim 18.

20. The agent according to any one of claim 12, 14 or 17, wherein said agent is selected from an antibody, aptamer, photoaptamer, protein, polypeptide, peptide, nucleic acid, peptidomimetic and small molecule.

21. A composition or formulation comprising the agent according to any one of claim 12, 14, 17 or 20 and one or more additional components, preferably wherein the composition or formulation is a pharmaceutical composition or formulation and said one or more additional components are one or more pharmaceutically acceptable carriers.

22. A kit of parts comprising the agent according to any one of claim 12, 14, 17 or 20.

23. The agent according to any one of claim 12, 14, 17 or 20, for use as a medicament, preferably for use in the treatment of a disease or condition associated with a retrovirus, preferably wherein the disease or condition associated with a retrovirus is a disease or condition associated with a human retrovirus including human pathogenic and non-pathogenic retrovirus, more preferably HIV or HTLV.

24. A method for diagnosing, predicting and/or prognosticating a disease or condition associated with a retrovirus in a subject, preferably wherein the disease or condition associated with a retrovirus is a disease or condition associated with a human retrovirus including human pathogenic and non-pathogenic retrovirus, more preferably HIV or HTLV, characterised in that the examination phase of the method comprises determining or measuring the structure, activity and/or level of:
the isolated complex according to any one of claim 1 to 4 or an endogenous complex comprising the first and second proteins as defined in any one of claims 1 to 4; and/or
the host interactor protein as defined in claim 17, preferably any such endogenous host interactor protein.

25. The method according to claim 24 comprising:
(a) determining or measuring the structure, activity and/or level of said complex and/or said host interactor protein in a sample from the subject;
(b) comparing the structure, activity and/or level of said complex and/or said host interactor protein determined or measured in (a) with a reference value of the structure, activity and/or level of said complex and/or said host interactor protein, said reference value representing a known diagnosis, prediction and/or prognosis of the disease or condition associated with a retrovirus;
(c) finding a deviation or no deviation of the structure, activity and/or level of said complex and/or said host interactor protein determined or measured in (a) from the reference value;
(d) attributing said finding of deviation or no deviation to a particular diagnosis, prediction and/or prognosis of the disease or condition associated with a retrovirus in the subject.

26. A method for modulating HIV Tat-mediated transactivation of HIV viral promoter sequences or HTLV Tax-mediated transactivation of HTLV viral promoter sequences, comprising modulating the activity and/or level of a protein selected from TRAF2, LNX2, MIZF, TSC22D4, BHLHB2, C10orf3, C16orf33, Clorf94, C20orf141, C8orf32, CCDC24, CRSP9, CRX, DAZAP2, DGCR6L, DHRS10, DIPA, DLAT, DLX2, DVL2, FANCG, FATE1, FLJ10726, FLJ22471, FTH1, FXR2, GADD45GIP1, GCC1, GOPC, HOXA3, HOXB9, HOXD3, HSFY1, KCTD1, KIAA1949, KIF9, KLC3, KRT15, KRT8, LENG1, LOC391257, LOC541468, LOC91661, LZTS2, MAD1L1, MINA, MLLT11, MRPS6, MYEF2, MYST2, NEFL, NIF3L1, NKAP, NOS3, OTX2, PCBP1, PDE9A, PIAS2, PSMF1, RNPS1, SF3A3, SP100, SPAG5, SPG21, TEX11, TFIP11, TRIP6, TTC23, UBE2I, ZMAT1, ZNF614 or a functional fragment or variant of any one thereof.

27. The method according to claim 26, wherein the protein is selected from TRAF2, LNX2, MIZF, TSC22D4, DLX2, FLJ10726, HOXA3, KRT8, LOC391257, LZTS2, MAD1L1, SF3A3, SPAG5, SPG21, TFIP11 or a functional fragment or variant of any one thereof.

28. A method for identifying interactors of a plurality of query proteins, wherein said query proteins comprise a subgroup of two or more proteins which are structurally and/or functionally similar or equivalent, the method comprising steps:
(a) screening of a plurality of target proteins to identify interactors of the query proteins; and
(b) where a given target protein is identified in step (a) as an interactor of a query protein from said subgroup of query proteins, testing the presence or absence of an interaction between said target protein and one or more or preferably all remaining query proteins from said subgroup of query proteins.

29. The method of claim 28, wherein the screening step (a) and testing of step (b) are performed using a yeast two-hybrid based method.
